SEQUENCE LISTING (116 sequences)

SEQ ID NO: 1 - protein, 217 aa, Homo sapiens
  MISC_FEATURE (1)..(217): Exemplary Human IgG1 CH2-CH3 Domain
  MISC_FEATURE (217)..(217): Xaa is lysine (K) or is absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 2 - protein, 216 aa, Homo sapiens
  MISC_FEATURE (1)..(216): Exemplary Human IgG2 CH2-CH3 Domain
  MISC_FEATURE (216)..(216): Xaa is lysine (K) or is absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 3 - protein, 217 aa, Homo sapiens
  MISC_FEATURE (1)..(217): Exemplary Human IgG3 CH2-CH3 Domain
  MISC_FEATURE (217)..(217): Xaa is lysine (K) or is absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 4 - protein, 217 aa, Homo sapiens
  MISC_FEATURE (1)..(217): Exemplary Human IgG4 CH2-CH3 Domain
  MISC_FEATURE (217)..(217): Xaa is lysine (K) or is absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 5 - protein, 937 aa, Homo sapiens
  MISC_FEATURE (1)..(937): Human ROR1 Isoform (NCBI Sequence NP_005003.2)
  MISC_FEATURE (1)..(29): Signal Sequence
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 6 - protein, 113 aa, Mus musculus
  MISC_FEATURE (1)..(113): VL Domain of Anti-ROR1 Antibody
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLG

SEQ ID NO: 7 - protein, 121 aa, Mus musculus
  MISC_FEATURE (1)..(121): VH Domain of Anti-ROR1 Antibody
QEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFDIWGQGTTVTVSS

SEQ ID NO: 8 - protein, 119 aa, Artificial Sequence
  Preferred Optimized VL Domain of Anti-ROR1 Antibody
  MISC_FEATURE (16)..(16): Xaa is Serine (S) or Glycine (G)
  MISC_FEATURE (19)..(19): Xaa is Lysine (K), Isoleucine (I) or Asparagine (N)
  MISC_FEATURE (49)..(49): Xaa is Lysine (K) or Asparagine (N)
  MISC_FEATURE (67)..(67): Xaa is Glycine (G) or is absent
  MISC_FEATURE (70)..(70): Xaa is Serine (S) or Isoleucine (I)
  MISC_FEATURE (76)..(76): Xaa is Arginine (R) or Tryptophan (W)
  MISC_FEATURE (97)..(97): Xaa is Tyrosine (Y) or Asparagine (N)
QLVLTQSPSASASLGXSVXLTCTLSSGHKTDTIDWYQQQPGKAPRYLMXLEGSGSYNKGSGVPDRFXSGXSSGADXYLTISSLQSEDEADYYCGTDXPGNYLFGGGTQLTSEQIDNVLG

SEQ ID NO: 9 - protein, 121 aa, Artificial Sequence
  Preferred Optimized VH Domain of Anti-ROR1 Antibody
  MISC_FEATURE (37)..(37): Xaa is Valine (V) or Isoleucine (I)
  MISC_FEATURE (64)..(64): Xaa is Valine (V) or Alanine (A)
  MISC_FEATURE (68)..(68): Xaa is Phenylalanine (F) or Leucine(L)
  MISC_FEATURE (77)..(77): Xaa is Asparagine (N), Aspartate (D) or Tyrosine (Y)
  MISC_FEATURE (97)..(97): Xaa is Alanine (A) or Threonine (T)
QEQLVESGGGLVQPGGSLRLSCAASGFTFSDYYMSWXRQAPGKGLEWVATIYPSSGKTYYADSXKGRXTISSDNAKXSLYLQMNSLRAEDTAVYYCXRDSYADDAALFDIWGQGTTVTVSS

SEQ ID NO: 10 - protein, 112 aa, Artificial Sequence
  Variant VL Domain of Anti-ROR1 Antibody (Anti-ROR1-VL(1))
QLVLTQSPSASASLGSSVKLTCTLSSGHKTDTIDWYQQQPGKAPRYLMKLEGSGSYNKGSGVPDRFSGSSSGADRYLTISSLQSEDEADYYCGTDYPGNYLFGGGTQLTVLG

SEQ ID NO: 11 - protein, 113 aa, Artificial Sequence
  Variant VL Domain of Anti-ROR1 Antibody (Anti-ROR1-VL(2))
QLVLTQSPSASASLGSSVKLTCTLSSGHKTDTIDWYQQQPGKAPRYLMKLEGSGSYNKGSGVPDRFGSGSSSGADWYLTISSLQSEDEADYYCGTDYPGNYLFGGGTQLTVLG

SEQ ID NO: 12 - protein, 113 aa, Artificial Sequence
  Variant VL Domain of Anti-ROR1 Antibody (Anti-ROR1-VL(3))
QLVLTQSPSASASLGSSVKLTCTLSSGHKTDTIDWYQQQPGKAPRYLMNLEGSGSYNKGSGVPDRFGSGSSSGADRYLTISSLQSEDEADYYCGTDYPGNYLFGGGTQLTVLG

SEQ ID NO: 13 - protein, 113 aa, Artificial Sequence
  Variant VL Domain of Anti-ROR1 Antibody (Anti-ROR1-VL(4))
QLVLTQSPSASASLGGSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLG

SEQ ID NO: 14 - protein, 113 aa, Artificial Sequence
  Variant VL Domain of Anti-ROR1 Antibody (Anti-ROR1-VL(5))
QLVLTQSPSASASLGSSVKLTCTLSSGHKTDTIDWYQQQPGKAPRYLMKLEGSGSYSKGSGVPDRFGSGSSSGADRYLTISSLQSEDEADYYCGTDYPGNYLFGGGTQLTVLG

SEQ ID NO: 15 - protein, 113 aa, Artificial Sequence
  Variant VL Domain of Anti-ROR1 Antibody (Anti-ROR1-VL(6))
QLVLTQSPSASASLGSSVKLTCTLSSGHKTDTIDWYQQQPGKAPRYLMKLEGSGSYNKGSGVPDRFGSGISSGADRYLTISSLQSEDEADYYCGTDYPGNYLFGGGTQLTVLG

SEQ ID NO: 16 - protein, 113 aa, Artificial Sequence
  Variant VL Domain of Anti-ROR1 Antibody (Anti-ROR1-VL(7))
QLVLTQSPSASASLGSSVILTCTLSSGHKTDTIDWYQQQPGKAPRYLMKLEGSGSYNKGSGVPDRFGSGSSSGADRYLTISSLQSEDEADYYCGTDYPGNYLFGGGTQLTVLG

SEQ ID NO: 17 - protein, 113 aa, Artificial Sequence
  Variant VL Domain of Anti-ROR1 Antibody (Anti-ROR1-VL(8))
QLVLTQSPSASASLGSSVNLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLG

SEQ ID NO: 18 - protein, 113 aa, Artificial Sequence
  Variant VL Domain of Anti-ROR1 Antibody (Anti-ROR1-VL(9))
QLVLTQSPSASASLGSSVKLTCTLSSGHKTDTIDWYQQQPGKAPRYLMKLEGSGSYTKGSGVPDRFGSGSSSGADRYLTISSLQSEDEADYYCGTDYPGNYLFGGGTQLTVLG

SEQ ID NO: 19 - protein, 113 aa, Artificial Sequence
  Variant VL Domain of Anti-ROR1 Antibody (Anti-ROR1-VL(10))
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDNPGNYLFGGGTQLTVLG

SEQ ID NO: 20 - protein, 113 aa, Artificial Sequence
  Variant VL Domain of Anti-ROR1 Antibody (Anti-ROR1-VL(11))
QLVLTQSPSASASLGSSVKLTCTLSSGHKTDTIDWYQQQPGKAPRYLMKLEGSGSYNKGSGVPDRFGSGSSSGADWYLTISSLQSEDEADYYCGTDNPGNYLFGGGTQLTVLG

SEQ ID NO: 21 - protein, 113 aa, Artificial Sequence
  Variant VL Domain of Anti-ROR1 Antibody (Anti-ROR1-VL(12))
QLVLTQSPSASASLGSSVKLTCTLSSGHKTDTIDWYQQQPGKAPRYLMKLEGSGSYNKGSGVPDRFGSGISSGADWYLTISSLQSEDEADYYCGTDYPGNYLFGGGTQLTVLG

SEQ ID NO: 22 - protein, 113 aa, Artificial Sequence
  Variant VL Domain of Anti-ROR1 Antibody (Anti-ROR1-VL(13))
QLVLTQSPSASASLGSSVKLTCTLSSGHKTDTIDWYQQQPGKAPRYLMKLEGSGSYNKGSGVPDRFGSGISSGADWYLTISSLQSEDEADYYCGTDNPGNYLFGGGTQLTVLG

SEQ ID NO: 23 - protein, 112 aa, Artificial Sequence
  Variant VL Domain of Anti-ROR1 Antibody (Anti-ROR1-VL(14))
QLVLTQSPSASASLGSSVKLTCTLSSGHKTDTIDWYQQQPGKAPRYLMKLEGSGSYNKGSGVPDRFSGSSSGADWYLTISSLQSEDEADYYCGTDYPGNYLFGGGTQLTVLG

SEQ ID NO: 24 - protein, 121 aa, Artificial Sequence
  Variant VH Domain of Anti-ROR1 Antibody (Anti-ROR1-VH(1))
QEQLVESGGGLVQPGGSLRLSCAASGFTFSDYYMSWVRQAPGKGLEWVATIYPSSGKTYYADSVKGRLTISSDNAKNSLYLQMNSLRAEDTAVYYCARDSYADDAALFDIWGQGTTVTVSS

SEQ ID NO: 25 - protein, 121 aa, Artificial Sequence
  Variant VH Domain of Anti-ROR1 Antibody (Anti-ROR1-VH(2))
QEQLVESGGGLVQPGGSLRLSCAASGFTFSDYYMSWVRQAPGKGLEWVATIYPSSGKTYYADSVKGRFTISSDNAKDSLYLQMNSLRAEDTAVYYCARDSYADDAALFDIWGQGTTVTVSS

SEQ ID NO: 26 - protein, 121 aa, Artificial Sequence
  Variant VH Domain of Anti-ROR1 Antibody (Anti-ROR1-VH(3))
QEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCTRDSYADDAALFDIWGQGTTVTVSS

SEQ ID NO: 27 - protein, 121 aa, Artificial Sequence
  Variant VH Domain of Anti-ROR1 Antibody (Anti-ROR1-VH(4))
QEQLVESGGGLVQPGGSLRLSCAASGFTFSDYYMSWVRQAPGKGLEWVATIYPSSGKTYYADSVKGRFTISSDNAKYSLYLQMNSLRAEDTAVYYCARDSYADDAALFDIWGQGTTVTVSS

SEQ ID NO: 28 - protein, 121 aa, Artificial Sequence
  Variant VH Domain of Anti-ROR1 Antibody (Anti-ROR1-VH(5))
QEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFAIWGQGTTVTVSS

SEQ ID NO: 29 - protein, 121 aa, Artificial Sequence
  Variant VH Domain of Anti-ROR1 Antibody (Anti-ROR1-VH(6))
QEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFYIWGQGTTVTVSS

SEQ ID NO: 30 - protein, 121 aa, Artificial Sequence
  Variant VH Domain of Anti-ROR1 Antibody (Anti-ROR1-VH(7))
QEQLVESGGGLVQPGGSLRLSCAASGFTFSDYYMSWVRQAPGKGLEWVATIYPSSGKTYYADSVKGRLTISSDNAKDSLYLQMNSLRAEDTAVYYCTRDSYADDAALFDIWGQGTTVTVSS

SEQ ID NO: 31 - protein, 121 aa, Artificial Sequence
  Variant VH Domain of Anti-ROR1 Antibody (Anti-ROR1-VH(8))
QEQLVESGGGLVQPGGSLRLSCAASGFTFSDYYMSWIRQAPGKGLEWVATIYPSSGKTYYADSAKGRLTISSDNAKDSLYLQMNSLRAEDTAVYYCTRDSYADDAALFDIWGQGTTVTVSS

SEQ ID NO: 32 - protein, 121 aa, Artificial Sequence
  Variant VH Domain of Anti-ROR1 Antibody (Anti-ROR1-VH(9))
QEQLVESGGGLVQPGGSLRLSCAASGFTFSDYYMSWIRQAPGKGLEWVATIYPSSGKTYYADSAKGRFTISSDNAKNSLYLQMNSLRAEDTAVYYCARDSYADDAALFDIWGQGTTVTVSS

SEQ ID NO: 33 - protein, 8 aa, Artificial Sequence
  Preferred Intervening Spacer Peptide (Linker 1)
GGGSGGGG

SEQ ID NO: 34 - protein, 6 aa, Artificial Sequence
  Preferred Cysteine-Containing Spacer Peptide (Linker 2)
GGCGGG

SEQ ID NO: 35 - protein, 4 aa, Artificial Sequence
  Alternative Spacer Peptide (Linker 2)
GGGS

SEQ ID NO: 36 - protein, 6 aa, Artificial Sequence
  Alternative Spacer Peptide (Linker 2)
LGGGSG

SEQ ID NO: 37 - protein, 11 aa, Artificial Sequence
  Alternative Spacer Peptide (Linker 2)
GGGSGGGSGGG

SEQ ID NO: 38 - protein, 5 aa, Artificial Sequence
  Alternative Spacer Peptide (Linker 2)
ASTKG

SEQ ID NO: 39 - protein, 6 aa, Artificial Sequence
  Alternative Spacer Peptide (Linker 2)
LEPKSS

SEQ ID NO: 40 - protein, 5 aa, Artificial Sequence
  Alternative Spacer Peptide (Linker 2)
APSSS

SEQ ID NO: 41 - protein, 7 aa, Artificial Sequence
  Heterodimer-Promoting Domain
GVEPKSC

SEQ ID NO: 42 - protein, 6 aa, Artificial Sequence
  Heterodimer-Promoting Domain
VEPKSC

SEQ ID NO: 43 - protein, 6 aa, Artificial Sequence
  Heterodimer-Promoting Domain
AEPKSC

SEQ ID NO: 44 - protein, 7 aa, Artificial Sequence
  Heterodimer-Promoting Domain
GFNRGEC

SEQ ID NO: 45 - protein, 6 aa, Artificial Sequence
  Heterodimer-Promoting Domain
FNRGEC

SEQ ID NO: 46 - protein, 28 aa, Artificial Sequence
  E-Coil Heterodimer-Promoting Domain
EVAALEKEVAALEKEVAALEKEVAALEK

SEQ ID NO: 47 - protein, 28 aa, Artificial Sequence
  K-Coil Heterodimer-Promoting Domain
KVAALKEKVAALKEKVAALKEKVAALKE

SEQ ID NO: 48 - protein, 28 aa, Artificial Sequence
  Cysteine-Containing E-Coil Heterodimer- Promoting Domain
EVAACEKEVAALEKEVAALEKEVAALEK

SEQ ID NO: 49 - protein, 28 aa, Artificial Sequence
  Cysteine-Containing K-Coil Heterodimer- Promoting Domain
KVAACKEKVAALKEKVAALKEKVAALKE

SEQ ID NO: 50 - protein, 46 aa, Streptococcus dysgalactiae
  MISC_FEATURE (1)..(46): Albumin-Binding Domain 3 (ABD3) of Protein G of Streptococcus strain G148
LAEAKVLANRELDKYGVSDYYKNLIDNAKSAEGVKALIDEILAALP

SEQ ID NO: 51 - protein, 46 aa, Artificial Sequence; identical to SEQ ID NO: 50
  Variant of Albumin-Binding Domain 3 (ABD3) of protein G of Streptococcus strain G148

SEQ ID NO: 52 - protein, 46 aa, Artificial Sequence
  Variant of Albumin-Binding Domain 3 (ABD3) of protein G of Streptococcus strain G148
LAEAKVLANRELDKYGVSDYYKNAANNAKTVEGVKALIAEILAALP

SEQ ID NO: 53 - protein, 46 aa, Artificial Sequence
  Variant of Albumin-Binding Domain 3 (ABD3) of protein G of Streptococcus strain G148
LAEAKVLANRELDKYGVSDYYKNLISNAKSVEGVKALIAEILAALP

SEQ ID NO: 54 - protein, 8 aa, Artificial Sequence
  Polypeptide Linker
APSSSPME

SEQ ID NO: 55 - protein, 16 aa, Artificial Sequence
  Polypeptide Linker
VEPKSADKTHTCPPCP

SEQ ID NO: 56 - protein, 16 aa, Artificial Sequence
  Polypeptide Linker
LEPKSADKTHTCPPCP

SEQ ID NO: 57 - protein, 10 aa, Artificial Sequence
  Polypeptide Linker
DKTHTCPPCP

SEQ ID NO: 58 - protein, 13 aa, Artificial Sequence
  Polypeptide Linker
GGGDKTHTCPPCP

SEQ ID NO: 59 - protein, 16 aa, Artificial Sequence
  Polypeptide Linker
LEPKSSDKTHTCPPCP

SEQ ID NO: 60 - protein, 15 aa, Homo sapiens
  MISC_FEATURE (1)..(15): Human IgG1 Hinge Region
EPKSCDKTHTCPPCP

SEQ ID NO: 61 - protein, 12 aa, Homo sapiens
  MISC_FEATURE (1)..(12): Human IgG2 Hinge Region
ERKCCVECPPCP

SEQ ID NO: 62 - protein, 12 aa, Homo sapiens
  MISC_FEATURE (1)..(12): Human IgG4 Hinge Region
ESKYGPPCPSCP

SEQ ID NO: 63 - protein, 12 aa, Artificial Sequence
  Variant of Human IgG4 Hinge Region Having Stabilizing S10P Substitution
ESKYGPPCPPCP

SEQ ID NO: 64 - protein, 15 aa, Artificial Sequence
  Intervening Spacer Peptide
GGGGSGGGGSGGGGS

SEQ ID NO: 65 - protein, 107 aa, Homo sapiens [sequence omitted]

SEQ ID NO: 66 - protein, 104 aa, Homo sapiens
  MISC_FEATURE (1)..(104): Human IgG CL Lambda Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 67 - protein, 98 aa, Homo sapiens
  MISC_FEATURE (1)..(98): Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 68 - protein, 98 aa, Homo sapiens
  MISC_FEATURE (1)..(98): human IgG2 CH1 Domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 69 - protein, 98 aa, Homo sapiens
  MISC_FEATURE (1)..(98): Human IgG4 CH1 Domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 70 - protein, 217 aa, Artificial Sequence
  CH2-CH3 Domains of Variant IgG1 Having Reduced or Abolished Effector Function
  MISC_FEATURE (217)..(217): Xaa is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 71 - protein, 217 aa, Artificial Sequence
  CH2-CH3 Domains of “Knob-Bearing” Variant IgG1 Having Reduced or Abolished Effector Function
  MISC_FEATURE (217)..(217): Xaa is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 72 - protein, 217 aa, Artificial Sequence
  CH2-CH3 Domains of “Hole-Bearing” Variant IgG1 Having Reduced or Abolished Effector Function
  MISC_FEATURE (217)..(217): Xaa is lysine (K) or is absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 73 - protein, 112 aa, Mus musculus
  MISC_FEATURE (1)..(112): VL Domain of Anti-CD2 Antibody “Lo-CD2a”
DVVLTQTPPTLLATIGQSVSISCRSSQSLLHSSGNTYLNWLLQRTGQSPQPLIYLVSKLESGVPNRFSGSGSGTDFTLKISGVEAEDLGVYYCMQFTHYPYTFGAGTKLELK

SEQ ID NO: 74 - protein, 118 aa, Mus musculus
  MISC_FEATURE (1)..(118): VH Domain of Anti-CD2 Antibody “Lo-CD2a”
EVQLQQSGPELQRPGASVKLSCKASGYIFTEYYMYWVKQRPKQGLELVGRIDPEDGSIDYVEKFKKKATLTADTSSNTAYMQLSSLTSEDTATYFCARGKFNYRFAYWGQGTLVTVSS

SEQ ID NO: 75 - protein, 110 aa, Mus musculus
  MISC_FEATURE (1)..(110): VL Domain of Anti-CD3 Antibody “CD3 mAb 1”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 76 - protein, 125 aa, Mus musculus
  MISC_FEATURE (1)..(125): VH Domain of Anti-CD3 Antibody “CD3 mAb 1”
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 77 - protein, 125 aa, Artificial Sequence
  VH Domain of Anti-CD3 Antibody “CD3 mAb 1” Having D65G Substitution
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 78 - protein, 125 aa, Mus musculus
  MISC_FEATURE (1)..(125): VH Domain of Anti-CD3 Antibody “CD3 mAb 1 Low”
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSS

SEQ ID NO: 79 - protein, 125 aa, Mus musculus
  MISC_FEATURE (1)..(125): VH Domain of Anti-CD3 Antibody “CD3 mAb 1 Fast”
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSS

SEQ ID NO: 80 - protein, 107 aa, Artificial Sequence
  VL Domain of Humanized Anti-CD3 Antibody OKT3
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 81 - protein, 119 aa, Artificial Sequence [sequence omitted]

SEQ ID NO: 82 - protein, 112 aa, Mus musculus
  MISC_FEATURE (1)..(112): VL Domain of Anti-CD8 Antibody OKT8
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKR

SEQ ID NO: 83 - protein, 120 aa, Mus musculus
  MISC_FEATURE (1)..(120): VH Domain of Anti-CD8 Antibody OKT8
QVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSS

SEQ ID NO: 84 - protein, 106 aa, Mus musculus
  MISC_FEATURE (1)..(106): VL Domain of Anti-CD8 Antibody TRX2
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 85 - protein, 121 aa, Mus musculus
  MISC_FEATURE (1)..(121): VH Domain of Anti-CD8 Antibody TRX2
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 86 - protein, 111 aa, Mus musculus
  MISC_FEATURE (1)..(111): VL Domain of Anti-CD16 Antibody “3G8”
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 87 - protein, 118 aa, Mus musculus
  MISC_FEATURE (1)..(118): VH Domain of Anti-CD16 Antibody “3G8”
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 88 - protein, 111 aa, Mus musculus
  MISC_FEATURE (1)..(111): VL Domain of Anti-CD16 Antibody “A9”
DIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

SEQ ID NO: 89 - protein, 117 aa, Mus musculus
  MISC_FEATURE (1)..(117): VH Domain of Anti-CD16 Antibody “A9”
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSS

SEQ ID NO: 90 - protein, 106 aa, Mus musculus
  MISC_FEATURE (1)..(106): VL Domain of Anti-TCR Antibody “BMA 031”
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 91 - protein, 120 aa, Mus musculus
  MISC_FEATURE (1)..(120): VH Domain of Anti-TCR Antibody “BMA 031”
QVQLVQSGAEVKKPGASVKVSCKASGYKFTSYVMHWVRQAPGQGLEWIGYINPYNDVTKYNEKFKGRVTITADKSTSTAYLQMNSLRSEDTAVHYCARGSYYDYDGFVYWGQGTLVTVSS

SEQ ID NO: 92 - protein, 108 aa, Mus musculus
  MISC_FEATURE (1)..(108): VL Domain of Anti-NKG2D Antibody KYK-1.0
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 93 - protein, 118 aa, Mus musculus
  MISC_FEATURE (1)..(118): VH Domain of Anti-NKG2D Antibody KYK-1.0
EVQLVESGGGVVQPGGSLRLSCAASGFTFSSYGMHWVRQAPGKGLEWVAFIRYDGSNKYYADSVKGRFTISRDNSKNTKYLQMNSLRAEDTAVYYCAKDRFGYYLDYWGQGTLVTVSS

SEQ ID NO: 94 - protein, 110 aa, Mus musculus
  MISC_FEATURE (1)..(110): VL Domain of Anti-NKG2D Antibody KYK-2.0
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 95 - protein, 121 aa, Mus musculus
  MISC_FEATURE (1)..(121): VH Domain of Anti-NKG2D Antibody KYK-2.0
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 96 - protein, 280 aa, Artificial Sequence
  First Polypeptide Chain of ROR1 x CD3 Bispecific Diabody “DART 25”
QLVLTQSPSASASLGSSVKLTCTLSSGHKTDTIDWYQQQPGKAPRYLMKLEGSGSYNKGSGVPDRFGSGSSSGADWYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 97 - protein, 273 aa, Artificial Sequence
  Second Polypeptide Chain of ROR1 x CD3 Bispecific Diabody “DART 25”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWVRQAPGKGLEWVATIYPSSGKTYYADSVKGRLTISSDNAKDSLYLQMNSLRAEDTAVYYCTRDSYADDAALFDIWGQGTTVTVSSGGCGGGEVAALEKEVAALEKEVAALEKEVAALEK

SEQ ID NO: 98 - protein, 509 aa, Artificial Sequence
  First Polypeptide Chain of ROR1 x CD3 Bispecific Diabody “DART-A”
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 272 aa, Artificial Sequence
  Second Polypeptide Chain of ROR1 x CD3 Bispecific Diabody “DART-A”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFDIWGQGTTVTVSSASTKGKVAACKEKVAALKEKVAALKEKVAALKE

SEQ ID NO: 100 - protein, 227 aa, Artificial Sequence
  Third Polypeptide Chain of ROR1 x CD3 Bispecific Diabody “DART-A”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 101 - protein, 508 aa, Artificial Sequence
  First Polypeptide Chain of ROR1 x CD3 Bispecific Diabody “DART-B”
QLVLTQSPSASASLGSSVKLTCTLSSGHKTDTIDWYQQQPGKAPRYLMKLEGSGSYNKGSGVPDRFSGSSSGADRYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 508 aa, Artificial Sequence
  First Polypeptide Chain of ROR1 x CD3 Bispecific Diabody “DART-C”
QLVLTQSPSASASLGSSVKLTCTLSSGHKTDTIDWYQQQPGKAPRYLMKLEGSGSYNKGSGVPDRFSGSSSGADWYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 272 aa, Artificial Sequence
  Second Polypeptide Chain of ROR1 x CD3 Bispecific Diabody “DART-C”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWVRQAPGKGLEWVATIYPSSGKTYYADSVKGRLTISSDNAKDSLYLQMNSLRAEDTAVYYCTRDSYADDAALFDIWGQGTTVTVSSASTKGKVAACKEKVAALKEKVAALKEKVAALKE

SEQ ID NO: 104 - protein, 272 aa, Artificial Sequence
  Second Polypeptide Chain of ROR1 x CD3 Bispecific Diabody “DART-D”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWIRQAPGKGLEWVATIYPSSGKTYYADSAKGRLTISSDNAKDSLYLQMNSLRAEDTAVYYCTRDSYADDAALFDIWGQGTTVTVSSASTKGKVAACKEKVAALKEKVAALKEKVAALKE

SEQ ID NO: 105 - protein, 509 aa, Artificial Sequence; identical to SEQ ID NO: 98
  First Polypeptide Chain of ROR1 x CD3 x CD8 Trispecific Binding Molecule “TRIDENT-A”

SEQ ID NO: 106 - protein, 272 aa, Artificial Sequence; identical to SEQ ID NO: 99
  Second Polypeptide Chain of ROR1 x CD3 x CD8 Trispecific Binding Molecule “TRIDENT-A”

SEQ ID NO: 107 - protein, 475 aa, Artificial Sequence
  Third Polypeptide Chain of ROR1 x CD3 x CD8 Trispecific Binding Molecule “TRIDENT-A”
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGCGTKVEIKGGGGSGGGGSGGGGSQVQLVESGGGVVQPGRSLRLSCAASGFTFSDFGMNWVRQAPGKCLEWVALIYYDGSNKFYADSVKGRFTISRDNSKNTLYLQMNSLRAEDTAVYYCAKPHYDGYYHFFDSWGQGTLVTVSSVEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 108 - protein, 451 aa, Artificial Sequence
  Third Polypeptide Chain of ROR1 x CD3 x CD8 Trispecific Binding Molecule “TRIDENT-B”
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 109 - protein, 213 aa, Artificial Sequence
  Fourth Polypeptide Chain of ROR1 x CD3 x CD8 Trispecific Binding Molecule “TRIDENT-B”
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 508 aa, Artificial Sequence; identical to SEQ ID NO: 102
  First Polypeptide Chain of ROR1 x CD3 x CD8 Trispecific Binding Molecule “TRIDENT-C”

SEQ ID NO: 111 - protein, 272 aa, Artificial Sequence; identical to SEQ ID NO: 104
  Second Polypeptide Chain of ROR1 x CD3 x CD8 Trispecific Binding Molecule “TRIDENT-C”

SEQ ID NO: 112 - protein, 280 aa, Artificial Sequence
  First Polypeptide Chain of ROR1 x CD3 Bispecific Diabody “DART-1”
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 113 - protein, 273 aa, Artificial Sequence
  Second Polypeptide Chain of ROR1 x CD3 Bispecific Diabody “DART-1”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFDIWGQGTTVTVSSGGCGGGEVAALEKEVAALEKEVAALEKEVAALEK

SEQ ID NO: 114 - protein, 279 aa, Artificial Sequence
  First Polypeptide Chain of ROR1 x CD3 Bispecific Diabody “DART-32”
QLVLTQSPSASASLGSSVKLTCTLSSGHKTDTIDWYQQQPGKAPRYLMKLEGSGSYNKGSGVPDRFSGSSSGADWYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 115 - protein, 273 aa, Artificial Sequence
  Second Polypeptide Chain of ROR1 x CD3 Bispecific Diabody “DART-33”
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWIRQAPGKGLEWVATIYPSSGKTYYADSAKGRLTISSDNAKDSLYLQMNSLRAEDTAVYYCTRDSYADDAALFDIWGQGTTVTVSSGGCGGGEVAALEKEVAALEKEVAALEKEVAALEK

SEQ ID NO: 116 - protein, 62 aa, Homo sapiens
  MISC_FEATURE (1)..(62): Human IgG3 Hinge Domain
ELKTPLGDTTHTCPRCPEPKSCDTPPPCPRCPEPKSCDTPPPCPRCPEPKSCDTPPPCPRCP

(19) United States
(12) Patent Application Publication
(10) Pub. No.: US 2017/0233472 A1
(43) Pub. Date: Aug. 17, 2017

(54) ROR1-Binding Molecules, and Methods of Use Thereof

(71) Applicant: MacroGenics, Inc., Rockville, MD (US)

(72) Inventors: Bhaswati Barat, Derwood, MD (US); Leslie S. Johnson, Darnestown, MD (US); Paul A. Moore, North Potomac, MD (US); Ralph Froman Alderson, North Potomac, MD (US); Ezio Bonvini, Potomac, MD (US)

(73) Assignee: MacroGenics, Inc., Rockville, MD (US)

(21) Appl. No.: 15/433,534

(22) Filed: Feb. 15, 2017

Related U.S. Application Data
(60) Provisional application No. 62/296,267, filed on Feb. 17, 2016.

(51) Int. Cl. C07K 16/28

(52) U.S. Cl. CPC C07K 16/28; C07K 16/2809; C07K 16/2815; C07K 2317/56; C07K 2317/92; C07K 2317/31

(57) ABSTRACT

The present invention is directed to optimized ROR1-binding molecules having enhanced affinity and superior ability to mediate redirected cytotoxicity of tumor cells relative to prior ROR1-binding molecules. More specifically, the invention relates to optimized ROR1-binding molecules that comprise Variable Light Chain and/or Variable Heavy Chain (VH) Domains that have been optimized for binding to an epitope present on the human ROR1 polypeptide so as to exhibit enhanced binding affinity for human ROR1 and/or a reduced immunogenicity upon administration to recipient subjects. The invention particularly pertains to bispecific, trispecific or multispecific ROR1-binding molecules, including bispecific diabodies, BiTEs, bispecific antibodies, trivalent binding molecules, etc. that comprise: (i) such optimized ROR1-binding Variable Domains and (ii) a domain capable of binding to an epitope of a molecule present on the surface of an effector cell. The invention is also directed to pharmaceutical compositions that contain any of such ROR1-binding molecules, and to methods involving the use of any of such ROR1-binding molecules in the treatment of cancer and other diseases and conditions.

### CROSS-REFERENCE TO RELATED APPLICATIONS

This application claims priority to U.S. Patent Appln. Ser. No. 62/296,267 (filed: Feb. 17, 2016; pending), which application is incorporated herein in its entirety.

### REFERENCE TO SEQUENCE LISTING

This application includes one or more Sequence Listings pursuant to 37 C.F.R. 1.821 et seq., which are disclosed in computer-readable media (file name: 1301_0139PCT_ST25.txt, created on Jan. 11, 2017, and having a size of 159,339 bytes), which file is herein incorporated by reference in its entirety.

### FIELD OF THE INVENTION

The present invention is directed to optimized ROR1-binding molecules having enhanced affinity and superior ability to mediate redirected cytotoxicity of tumor cells relative to prior ROR1-binding molecules. More specifically, the invention relates to optimized ROR1-binding molecules that comprise Variable Light Chain and/or Variable Heavy Chain (VH) Domains that have been optimized for binding to an epitope present on the human ROR1 polypeptide so as to exhibit enhanced binding affinity for human ROR1 and/or a reduced immunogenicity upon administration to recipient subjects. The invention particularly pertains to bispecific, trispecific or multispecific ROR1-binding molecules, including bispecific diabodies, BiTEs, bispecific antibodies, trivalent binding molecules, etc. that comprise: (i) such optimized ROR1-binding Variable Domains and (ii) a domain capable of binding to an epitope of a molecule present on the surface of an effector cell. The invention is also directed to pharmaceutical compositions that contain any of such ROR1-binding molecules, and to methods involving the use of any of such ROR1-binding molecules in the treatment of cancer and other diseases and conditions.

### BACKGROUND OF THE INVENTION

Receptor Tyrosine Kinase-Like Orphan Receptor 1 (“ROR1”) is a type I membrane protein belonging to the ROR subfamily of cell surface receptors (Masiakowski, P. et al. (1992). “A Novel Family Of Cell Surface Receptors With Tyrosine Kinase-Like Domain,” J. Biol. Chem. 267:26181-26190). ROR1 is an onco-embryonic antigen that is expressed by many tissues during embryogenesis, is absent from most mature tissues (Paganoni, S. et al. (2005) “Neurite Extension In Central Neurons: A Novel Role For The Receptor Tyrosine Kinases ROR1 And ROR2,” J. Cell Sci. 118:433-446) and is expressed in numerous blood and solid malignancies including ovarian, colon, lung, lymphoma, skin, pancreatic, testicular, bladder, uterus, prostate, adrenal, breast, and B-cell malignancies, as well as in some cancer stem cells (Zhang, S. et al. (2012) “The Onco-Embryonic Antigen ROR1 Is Expressed by a Variety of Human Cancers,” Am. J. Pathol. 6:1903-1910; Zhang, S. et al. (2012) “ROR1 Is Expressed In Human Breast Cancer And Associated With Enhanced Tumor-Cell Growth,” PLoS One 7:e31127; Daneshmanesh, A. H., et al. (2008) “ROR1, A Cell Surface Receptor Tyrosine Kinase Is Expressed In Chronic Lymphocytic Leukemia And May Serve As A Putative Target For Therapy,” Int. J. Cancer 123:1190-1195; Zhang, S., et al. (2014) “Ovarian Cancer Stem Cells Express ROR1, Which Can Be Targeted For Anti-Cancer-Stem-Cell Therapy,” Proc. Natl. Acad. Sci. (U.S.A.) 111:17266-71). ROR1 expression is associated with high-grade tumors exhibiting a less-differentiated morphology and is correlated with poor clinical outcomes (Zhang, S., et al. (2012) “The Onco-Embryonic Antigen ROR1 Is Expressed by a Variety of Human Cancers,” Am. J. Pathol. 6:1903-1910; Zhang, H. et al. (2014) “ROR1 Expression Correlated With Poor Clinical Outcome In Human Ovarian Cancer,” Sci. Rep. 4:5811, pp. 1-7).

In light of the restricted expression of the ROR1 onco-embryonic antigen, a number of different immuno-based strategies to target ROR1 have been explored including antibodies, antibody drug conjugates, chimeric antigen receptor (CAR) expressing T cells, and ROR1-targeted nanoparticles (Choi, M. Y., et al. (2015) “Pre-clinical Specificity and Safety of UC-961, a First-In-Class Monoclonal Antibody Targeting ROR1,” Clin Lymphoma Myeloma Leuk 15(Suppl):S167-5169; Daneshmanesh, A. H., et al. (2012) “Monoclonal Antibodies Against ROR1 Induce Apoptosis Of Chronic Lymphocytic Leukemia (CLL) cells,” Leukemia 26:1348-1355; Yang, J., et al. (2011) “Therapeutic Potential And Challenges Of Targeting Receptor Tyrosine Kinase ROR1 With Monoclonal Antibodies In B-Cell Malignancies,” PLoS One 6:e21018; Baskar, S., et al. (2012) “Targeting Malignant B Cells With An Immunotoxin Against ROR1,” MAbs 4:349-361; Berger, C., et al. (2015) “Safety Of Targeting ROR1 In Primates With Chimeric Antigen Receptor-Modified T Cells,” Cancer Immunol. Res. 3:206-216; Hudecek, M., et al. (2010) “The B-Cell Tumor-Associated Antigen ROR1 Can Be Targeted With T Cells Modified To Express A ROR1-Specific Chimeric Antigen Receptor,” Blood. 116:4532-4541; Mani, R., et al. (2015) “Tumor Antigen ROR1 Targeted Drug Delivery Mediated Selective Leukemic But Not Normal B-Cell Cytotoxicity In Chronic Lymphocytic Leukemia,” Leukemia 29:346-355).

However, despite all prior advances, a need remains for high affinity ROR1-binding molecules having enhanced anti-tumor activity and/or reduced immunogenicity. The present invention addresses this need and the need for improved therapeutics for cancer.

### SUMMARY OF THE INVENTION

The present invention is directed to optimized ROR1-binding molecules having enhanced affinity and superior ability to mediate redirected cytotoxicity of tumor cells relative to prior ROR1-binding molecules. More specifically, the invention relates to optimized ROR1-binding molecules that comprise Variable Light Chain and/or Variable Heavy Chain (VH) Domains that have been optimized for binding to an epitope present on the human ROR1 polypeptide so as to exhibit enhanced binding affinity for human ROR1 and/or a reduced immunogenicity upon administration to recipient subjects. The invention particularly pertains to bispecific, trispecific or multispecific ROR1-binding molecules, including bispecific diabodies, BiTEs, bispecific antibodies, trivalent binding molecules, etc. that comprise: (i) such optimized ROR1-binding Variable Domains and (ii) a domain capable of binding to an epitope of a molecule present on the surface of an effector cell. The invention is also directed to pharmaceutical compositions that contain any of such ROR1-binding molecules, and to methods involving the use of any of such ROR1-binding molecules in the treatment of cancer and other diseases and conditions.

In detail, the invention provides such an optimized ROR1-binding molecule that comprises a Variable Light Chain Domain and a Variable Heavy Chain Domain, wherein the Variable Light Chain Domain has the amino acid sequence of SEQ ID NO:8: (CDR_{L} residues are shown underlined):

QLVLTQSPSASASLGX₁SVX₂LTCTLSSGHKTDTID
WYQQQPGKAPRYLMX₃LEGSGSYNKGSGVPDRFX₄S
GX₅SSGADX₆YLTISSLQSEDEADYYCGTDX₇
PGNYLFGGGTQLTVLG

wherein X₆ is W, and wherein:

(a) X₁ is S or G, X₂ is K, I or N, X₃ is K or N, X₄ is G or is absent, X₅ is S or I, X₇ is Y or N;

(b) X₁ is S, X₂ is K, X₃ is K, X₄ is G or is absent, X₅ is S, and X₇ is N;

(c) X₁ is S, X₂ is K, X₃ is K, X₄ is G or is absent, X₅ is I, and X₇ is Y;

(d) X₁ is S, X₂ is K, X₃ is K, X₄ is G or is absent, X₅ is I, and X₇ is N; or

(e) X₁ is S, X₂ is K, X₃ is K, X₄ is G or is absent, X₅ is S, and X₇ is Y.

The invention further provides an optimized ROR1-binding molecule that comprises a Variable Light Chain Domain and a Variable Heavy Chain Domain, wherein the Variable Heavy Chain Domain has the amino acid sequence of SEQ ID NO:9: (CDR_{H} residues are shown underlined):

QEQLVESGGGLVQPGGSLRLSCAASGFTFSDYYMSWX₁RQAPG
KGLEWVATIYPSSGKTYYADSX₂KGRX₃TISSDNAK
X₄SLYLQMNSLRAEDTAVYYCX₅RDSYADDAALFDI
WGQGTTVTVSS

wherein:

(a) X₁ is V or I, X₂ is V or A, X₃ is L, X₄ is N, D, or Y, and X₅ is A or T;

(b) X₁ is V or I, X₂ is V or A, X₃ is F or L, X₄ is D or Y, and X₅ is A or T;

(c) X₁ is V or I, X₂ is V or A, X₃ is F or L, X₄ is N, D, or Y, and X₅ is T;

(d) X₁ is V or I, X₂ is V or A, X₃ is L, X₄ is N, and X₅ is A;

(e) X₁ is V or I, X₂ is V or A, X₃ is F, X₄ is D, and X₅ is A;

(f) X₁ is V or I, X₂ is V or A, X₃ is F, X₄ is N, and X₅ is T;

(g) X₁ is V or I, X₂ is V or A, X₃ is L, X₄ is D, and X₅ is T;

(h) X₁ is I, X₂ is A, X₃ is F or L, X₄ is N, D or Y, and X₅ is A or T;

(i) X₁ is I, X₂ is A, X₃ is F, X₄ is N, and X₅ is A;

(j) X₁ is I, X₂ is A, X₃ is L, X₄ is N, and X₅ is A;

(k) X₁ is I, X₂ is A, X₃ is F, X₄ is D, and X₅ is A;

(l) X₁ is I, X₂ is A, X₃ is F, X₄ is N, and X₅ is T; or

(m) X₁ is I, X₂ is A, X₃ is L, X₄ is D, and X₅ is T.

The invention further concerns the embodiments of such a ROR1-binding molecule wherein:
(a) the VL Domain of such molecule comprises the amino acid sequence of SEQ ID NO:11, SEQ ID NO:20, SEQ ID NO:21, SEQ ID NO:22, or SEQ ID NO:23; and
(b) the VH Domain of such molecule comprises the amino acid sequence of SEQ ID NO:24, SEQ ID NO:25, SEQ ID NO:26, SEQ ID NO:30, SEQ ID NO:31, or SEQ ID NO:32.

The invention further concerns the embodiment of such ROR1-binding molecules, wherein the molecule is an antibody or an epitope-binding fragment thereof. In invention also concerns the embodiments of such a ROR1-binding molecule, wherein the molecule is a bispecific antibody or a diabody, especially a diabody, or diabody complex, that comprises two, three, four or five polypeptide chains each having an N-terminus and a C-terminus in which such polypeptide chains are associated together via one or more covalent, and especially one or more covalent disulfide, bonds. The invention additionally concerns the embodiment of such ROR1-binding molecules wherein the molecule is a trivalent binding molecule, and especially wherein the trivalent binding molecule is a covalently bonded complex that comprises three, four, five, or more polypeptide chains. The invention further concerns the embodiment of such a ROR1-binding molecule, wherein the molecule comprises an Fc Region. The invention additionally concerns the embodiment of such ROR1-binding molecules wherein the molecule is a diabody and comprises an Albumin-Binding Domain, and especially a deimmunized Albumin-Binding Domain.

The invention further concerns the embodiments of all such ROR1-binding molecules that additionally comprise an Fc Region, and especially wherein the Fc Region is a variant Fc Region that comprises one or more amino acid modifications that reduces the affinity of the variant Fc Region for an FcγR and/or enhances the serum half-life of the ROR1-binding molecule, and more particularly, wherein the modifications comprise at least one substitution selected from the group consisting of:

(a) L234A;

(b) L235A;

(c) L234A and L235A;

(d) M252Y; M252Y and S254T;

(e) M252Y and T256E;

(f) M252Y, S254T and T256E; and

(g) K288D and H435K;

wherein the numbering is that of the EU index as in Kabat.

The invention further concerns the embodiment of such ROR1-binding molecules, wherein the molecule is bispecific, and particularly concerns the embodiment wherein the molecule comprises two epitope-binding sites capable of immunospecific binding to an epitope of ROR1 and two epitope-binding sites capable of immunospecific binding to an epitope of a molecule present on the surface of an effector cell, or the embodiment wherein the molecule comprises one epitope-binding site capable of immunospecific binding to an epitope of ROR1 and one epitope-binding site capable of immunospecific binding to an epitope of a molecule present on the surface of an effector cell.

The invention additionally concerns the embodiment of such ROR1 binding molecules wherein the molecule is a trivalent binding molecule, and particularly concerns the embodiments wherein the molecule comprises, one epitope-binding site capable of immunospecific binding to an epitope of ROR1, one epitope-binding site capable of immunospecific binding to an epitope of a first molecule present on the surface of an effector cell; and one epitope-binding site capable of immunospecific binding to an epitope of a second molecule present on the surface of an effector cell, wherein such first and second molecules are not ROR1.

The invention further concerns the embodiment of such ROR1-binding molecules, wherein the molecule is capable of simultaneously binding to ROR1 and to a second epitope, and particularly concerns the embodiment wherein the second epitope is an epitope of a second molecule present on the surface of an effector cell (especially wherein the second epitope is an epitope of CD2, CD3, CD8, CD16, TCR, or NKG2D, and most particularly wherein the second epitope is an epitope of CD3). The invention additionally concerns the embodiment of such ROR1-binding molecules, wherein the effector cells is a cytotoxic T-cell or a Natural Killer (NK) cell. The invention additionally concerns the embodiment of such ROR1-binding molecules, wherein the molecule is also capable of binding a third epitope, and particularly concerns the embodiment wherein the third epitope is an epitope of CD8. The invention further concerns the embodiments of such molecules wherein molecule mediates coordinated binding of a cell expressing ROR1 and a cytotoxic T cell.

The invention further concerns the embodiment of such ROR1-binding molecules, wherein the molecule comprises a first polypeptide chain, a second polypeptide chain and a third polypeptide chain, and wherein:
(a) said a first polypeptide chain comprising SEQ ID NO:98, SEQ ID NO:101, or SEQ ID NO:102;
(b) said second polypeptide chain comprising SEQ ID NO:99, SEQ ID NO:103, or SEQ ID NO:104; and
(c) said third polypeptide chain comprises SEQ ID NO:100.

The invention further provides pharmaceutical compositions comprising an effective amount of any of the above-described ROR1-binding molecules and a pharmaceutically acceptable carrier, excipient or diluent.

The invention is additionally directed to the use of any of the above-described ROR1-binding molecules in the treatment of a disease or condition associated with or characterized by the expression of ROR1, or in a method of treating a disease or condition characterized by the expression of ROR1, particularly wherein the disease or condition associated with or characterized by the expression of ROR1 is cancer, and more particularly, wherein the cancer is selected from the group consisting of: an adrenal gland tumor, an AIDS-associated cancer, an alveolar soft part sarcoma, an astrocytic tumor, an adrenal cancer, a bladder cancer, a bone cancer, a brain and spinal cord cancer, a metastatic brain tumor, a B-cell cancer, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

### BRIEF DESCRIPTION OF THE DRAWINGS

FIG. 1 provides a schematic of a representative covalently bonded diabody having two epitope-binding sites composed of two polypeptide chains, each having an E-coil or K-coil Heterodimer-Promoting Domain (alternative Heterodimer-Promoting Domains are provided below). A cysteine residue may be present in a linker and/or in the Heterodimer-Promoting Domain as shown in FIG. 3B. VL and VH Domains that recognize the same epitope are shown using the same shading or fill pattern.

FIG. 2 provides a schematic of a representative covalently bonded diabody molecule having two epitope-binding sites composed of two polypeptide chains, each having a CH2 and CH3 Domain, such that the associated chains form all or part of an Fc Region. VL and VH Domains that recognize the same epitope are shown using the same shading or fill pattern.

FIGS. 3A-3C provide schematics showing representative covalently bonded tetravalent diabodies having four epitope-binding sites composed of two pairs of polypeptide chains (i.e., four polypeptide chains in all). One polypeptide of each pair possesses a CH2 and CH3 Domain, such that the associated chains form all or part of an Fc Region. VL and VH Domains that recognize the same epitope are shown using the same shading or fill pattern. The two pairs of polypeptide chains may be same. In such embodiments, wherein the two pairs of polypeptide chains are the same and the VL and VH Domains recognize different epitopes (as shown in FIGS. 3A-3B), the resulting molecule possesses four epitope-binding sites and is bispecific and bivalent with respect to each bound epitope. In such embodiments, wherein the VL and VH Domains recognize the same epitope (e.g., the same VL Domain CDRs and the same VH Domain CDRs are used on both chains) the resulting molecule possesses four epitope-binding sites and is monospecific and tetravalent with respect to a single epitope. Alternatively, the two pairs of polypeptides may be different. In such embodiments, wherein the two pairs of polypeptide chains are different and the VL and VH Domains of each pair of polypeptides recognize different epitopes (as shown by the different shading and patterns in FIG. 3C), the resulting molecule possesses four epitope-binding sites and is tetraspecific and monovalent with respect to each bound epitope. FIG. 3A shows an Fc Region-containing diabody which contains a peptide Heterodimer-Promoting Domain comprising a cysteine residue. FIG. 3B shows an Fc Region-containing diabody, which contains E-coil and K-coil Heterodimer-Promoting Domains comprising a cysteine residue and a linker (with an optional cysteine residue). FIG. 3C, shows an Fc-Region-Containing diabody, which contains antibody CH1 and CL domains.

FIGS. 4A and 4B provide schematics of a representative covalently bonded diabody molecule having two epitope-binding sites composed of three polypeptide chains. Two of the polypeptide chains possess a CH2 and CH3 Domain, such that the associated chains form all or part of an Fc Region. The polypeptide chains comprising the VL and VH Domain further comprise a Heterodimer-Promoting Domain. VL and VH Domains that recognize the same epitope are shown using the same shading or fill pattern.

FIG. 5 provides the schematics of a representative covalently bonded diabody molecule having four epitope-binding sites composed of five polypeptide chains. Two of the polypeptide chains possess a CH2 and CH3 Domain, such that the associated chains form an Fc Region that comprises all or part of an Fc Region. The polypeptide chains comprising the linked VL and VH Domains further comprise a Heterodimer-Promoting Domain. VL and VH Domains that recognize the same epitope are shown using the same shading or fill pattern.

FIGS. 6A-6F provide schematics of representative Fc Region-containing trivalent binding molecules having three epitope-binding sites. FIGS. 6A and 6B, respectively, illustrate schematically the domains of trivalent binding molecules comprising two diabody-type binding domains and a Fab-type binding domain having different domain orientations in which the diabody-type binding domains are N-terminal or C-terminal to an Fc Region. The molecules in FIGS. 6A and 6B comprise four chains. FIGS. 6C and 6D, respectively, illustrate schematically the domains of trivalent binding molecules comprising two diabody-type binding domains N-terminal to an Fc Region, and a Fab-type binding domain in which the light chain and heavy chain are linked via a polypeptide spacer, or an scFv-type binding domain. The trivalent binding molecules in FIGS. 6E and 6F, respectively, illustrate schematically the domains of trivalent binding molecules comprising two diabody-type binding domains C-terminal to an Fc Region, and a Fab-type binding domain in which the light chain and heavy chain are linked via a polypeptide spacer, or an scFv-type binding domain. The trivalent binding molecules in FIGS. 6C-6F comprise three chains. VL and VH Domains that recognize the same epitope are shown using the same shading or fill pattern.

FIGS. 7A-7B depict the amino acid sequences of the non-optimized anti-ROR1-VL Domain (FIG. 7A, SEQ ID NO:6) and non-optimized VH Domain (FIG. 7B, SEQ ID NO:7) of a parental ROR1-binding molecule. Underlining indicates CDR residues, Boxes indicate residues that are mutated in the sequences of preferred optimized anti-ROR1 binding molecules; Kabat positions are indicated with arrows and by the numbering below the sequence, sequential amino acid residue numbering is indicated above the sequences.

FIGS. 8A-8B show the ability of the ROR1×CD3 bispecific two chain covalently bonded diabodies: DART-1, DART-2, DART-16 and DART-20, to mediate redirected cell killing of JIMT-1 breast carcinoma cells as measured by cell-associated luciferase activity (FIG. 8A) or the release of lactate dehydrogenase (LDH) into the culture medium upon cell lysis (FIG. 8B).

FIGS. 9A-9B show the ability of the ROR1×CD3 bispecific two chain covalently bonded diabodies DART-1, DART-14, DART-15, DART-22 and DART-23 to mediate redirected cell killing of JIMT-1 breast carcinoma cells measured by cell-associated luciferase activity (FIG. 9A) or the release of lactate dehydrogenase (LDH) into the culture medium upon cell lysis (FIG. 9B).

FIGS. 10A-10C show the ability of the ROR1×CD3 bispecific two chain covalently bonded diabodies DART-1, DART-22, and DART-25 to mediate redirected cell killing of JIMT-1 breast carcinoma cells (FIG. 10A), HBL-2 mantle cell lymphoma cells (FIG. 10B), or Jeko-1 mantle cell lymphoma cells (FIG. 10C) as measured by the release of lactate dehydrogenase (LDH) into the culture medium upon cell lysis.

FIGS. 11A-11B show the dual antigen binding ability of ROR1×CD3 bispecific two and three chain diabodies using a sandwich ELISA. The binding curves for the two chain diabodies DART-1 and DART-A are shown in FIG. 11A (binding is a function of absorbance at 450 nm). The mean values of the binding curves for the three chain diabodies DART-A, DART-B, and DART-C are shown in FIG. 11B.

FIGS. 12A-12D are tracings of FACS cytometry profiles and show the ability of the ROR1×CD3 bispecific three chain diabody DART-D to bind to ROR1-expressing cancer cell lines HOP-92 (FIG. 12A), PC-3 (FIG. 12B) and HBL-2 (FIG. 12C), and to CD3-expressing human primary T-cells (FIG. 12D) by FACS.

FIGS. 13A-13D show the ability of the ROR1×CD3 bispecific two and three chain diabodies DART-1 and DART-A to mediate redirected cell killing of JIMT-1 breast carcinoma cells (FIG. 13A), A549 lung cancer cells (FIG. 13B), HBL-2 mantle cell lymphoma cells (FIG. 13C), and RECA0201 cancer stem cells (FIG. 13D). Cytotoxicity is measured by the release of lactate dehydrogenase (LDH) into the culture medium upon cell lysis.

FIGS. 14A-14B show the ability of the three chain diabodies DART-A, DART-C, and DART-D to mediate redirected cell killing of JIMT-1 breast carcinoma cells (FIG. 14A), and NCI-H1975 cells (FIG. 14B). Cytotoxicity is measured by the release of lactate dehydrogenase (LDH) into the culture medium upon cell lysis.

FIGS. 15A-15H show the ability of the representative three chain ROR1×CD3 bispecific diabody DART-D to mediate redirected cell killing of HBL-2 B-cell lymphoma cells (FIG. 15A); HOP-92 lung adenocarcinoma cells (FIG. 15B); PC-3M prostate cancer cells (FIG. 15C); Daoy medulloblastoma cells (FIG. 15D); and Saos-2 bone osteosarcoma (FIG. 15E), U-2 OS bone osteosarcoma (FIG. 15F), and MG-63 bone osteosarcoma (FIG. 15G). As expected, DART-D did not mediate redirected cell killing of ROR1 negative CHO cells (FIG. 15H). Cytotoxicity is measured by the release of lactate dehydrogenase (LDH) into the culture medium upon cell lysis.

FIGS. 16A-16B show the ability of the representative three chain ROR1×CD3 bispecific diabody DART-D to mediate cytoxicity in the presence of target NCI-H1975 cells and PBMCs (FIG. 16A), no cytoxicity was observed in the presence of PBMCs along (FIG. 16B). Cytotoxicity is measured by the release of lactate dehydrogenase (LDH) into the culture medium upon cell lysis.

FIGS. 17A-17D show the ability of the representative three chain ROR1×CD3 bispecific diabody DART-D to up regulate CD69 (FIGS. 17A-17B) and CD25 (FIGS. 17C-17D), T-cell activation markers, on CD4+ (FIGS. 17A and 17C) and CD8+ T-cell subsets (FIGS. 17B and 17D) in a dose-dependent manner in the presence of ROR1-expressing NCI-H1975 target cells.

FIGS. 18A-18F show IFN-γ (FIG. 18A), TNF-α (FIG. 18B), IL-10 (FIG. 18C), IL-6 (FIG. 18D), IL-4 (FIG. 18E) and IL-2 (FIG. 18F), cytokine levels in culture supernatants of PBMCs treated with DART-D (closed squares) or the negative control diabody (open diamonds) in the presence of ROR1-expressing target cells (closed symbols), or PBMCs alone were treated with DART-D (open squares) or the negative control diabody (open circles).

FIGS. 19A-19B show the ability of the ROR1×CD3 bispecific diabodies DART-1 (FIG. 19A) and DART-A (FIG. 19B) to prevent or inhibit tumor growth or development of HBL-2 mantle cell lymphoma cells in vivo relative to a vehicle control in a murine co-mix xenograft model.

FIGS. 20A-20B show the ability of the ROR1×CD3 bispecific diabodies DART-A (FIG. 20A) and DART-D (FIG. 20B) to prevent or inhibit tumor growth or development of HOP-92 lung adenocarcinoma cells in vivo relative to a vehicle control in a murine PBMC-reconstituted xenograft model.

FIGS. 21A-21B show the ability of the ROR1×CD3 bispecific diabodies DART-B (FIG. 21A) and DART-D (FIG. 21B) to prevent or inhibit tumor growth or development of NCI-H1975 lung cancer cells in vivo relative to a vehicle control in a PBMC-reconstituted murine xenograft model.

FIG. 22 shows the ability of the ROR1×CD3 bispecific diabody DART-B to prevent or inhibit tumor growth or development of REC1 mantle cancer cells in vivo relative to a vehicle control in a co-mix murine xenograft model.

FIG. 23 shows the ability of the ROR1×CD3 bispecific diabody DART-D to prevent or inhibit tumor growth or development of REC1 mantle cancer cells in vivo relative to a vehicle control in a PBMC-reconstituted murine xenograft model.

FIG. 24 shows the ability of the ROR1×CD3 bispecific diabody DART-D to prevent or inhibit tumor growth or development of DAOY desmoplastic cerebellar medulloblastoma cells in vivo relative to a vehicle control in a murine co-mix xenograft model.

FIGS. 25A-25C shows the ability of the bispecific ROR1×CD3 three chain diabody DART-A, and the trispecific ROR1×CD3×CD8 trivalent binding molecules TRIDENT-A and TRIDENT-B to mediate redirected cell killing of JIMT-1 breast cancer cells (FIG. 25A), NCI-H1975 cells (FIG. 25B), and Calu-3 lung adenocarcinoma cells (FIG. 25C). Cytotoxicity is measured by the release of lactate dehydrogenase (LDH) into the culture medium upon cell lysis.

### DETAILED DESCRIPTION OF THE INVENTION

The present invention is directed to optimized ROR1-binding molecules having enhanced affinity and superior ability to mediate redirected cytotoxicity of tumor cells relative to prior ROR1-binding molecules. More specifically, the invention relates to optimized ROR1-binding molecules that comprise Variable Light Chain and/or Variable Heavy Chain (VH) Domains that have been optimized for binding to an epitope present on the human ROR1 polypeptide so as to exhibit enhanced binding affinity for human ROR1 and/or a reduced immunogenicity upon administration to recipient subjects. The invention particularly pertains to bispecific, trispecific or multispecific ROR1-binding molecules, including bispecific diabodies, BiTEs, bispecific antibodies, trivalent binding molecules, etc. that comprise: (i) such optimized ROR1-binding Variable Domains and (ii) a domain capable of binding to an epitope of a molecule present on the surface of an effector cell. The invention is also directed to pharmaceutical compositions that contain any of such ROR1-binding molecules, and to methods involving the use of any of such ROR1-binding molecules in the treatment of cancer and other diseases and conditions.

### I. Antibodies and Their Binding Domains

The antibodies of the present invention are immunoglobulin molecules capable of specific binding to a target, such as a carbohydrate, polynucleotide, lipid, polypeptide, etc., through at least one antigen recognition site, located in the Variable Domain of the immunoglobulin molecule. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG_{2,} IgG_{3,} IgG_{4,} IgA₁ and IgA₂) or subclass. Antibodies are capable of “immunospecifically binding” to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens”. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Over 200 antibody-based drugs have been approved for use or are under development.

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the monospecific or multispecific (e.g., bispecific, trispecific and tetraspecific) molecules of the invention as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody, while swapping the non-human remainder of the antibody with human antibody sequences.

Natural antibodies (such as IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Region. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide). The Variable Domains of an IgG molecule consist of the complementarity determining regions (“CDR”), which contain the residues in contact with epitope, and non-CDR segments, referred to as framework segments (“FR”), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe, etc.), or is another type of protein. Accordingly, as used herein, the term “epitope-binding fragment” denotes a fragment of a molecule capable of immunospecifically binding to an epitope. An epitope-binding fragment may contain any 1, 2, 3, 4, or 5 the CDR Domains of an antibody, or may contain all 6 of the CDR Domains of an antibody and, although capable of immunospecifically binding to such epitope, may exhibit an immunospecificity, affinity or selectivity toward such epitope that differs from that of such antibody. Preferably, however, an epitope-binding fragment will contain all 6 of the CDR Domains of such antibody. An epitope-binding fragment of an antibody may be a single polypeptide chain (e.g., an scFv), or may comprise two or more polypeptide chains, each having an amino terminus and a carboxy terminus (e.g., a diabody, a Fab fragment, an Fab₂ fragment, etc.). Unless specifically noted, the order of domains of the protein molecules described herein is in the “N-terminal to C-Terminal” direction.

The invention particularly encompasses single-chain Variable Domain fragments (“scFv”) comprising an optimized anti-ROR1-VL and/or VH Domain of this invention and multispecific binding molecules comprising the same. Single-chain Variable Domain fragments comprise VL and VH Domains that are linked together using a short “Linker” peptide. Such Linkers can be modified to provide additional functions, such as to permit the attachment of a drug or to permit attachment to a solid support. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The invention also particularly encompasses optimized ROR1-binding molecules comprising an anti-ROR1-VL and/or VH Domain of a humanized antibody. The term “humanized” antibody refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The polynucleotide sequence of the variable domains of such antibodies may be used for genetic manipulation to generate such derivatives and to improve the affinity, or other characteristics of such antibodies. The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

The epitope-binding site may comprise either a complete Variable Domain fused onto Constant Domains or only the complementarity determining regions (CDRs) of such Variable Domain grafted to appropriate framework regions. Epitope-binding sites may be wild-type or modified by one or more amino acid substitutions. This eliminates the constant region as an immunogen in human individuals, but the possibility of an immune response to the foreign variable domain remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable domains as well so as to reshape them as closely as possible to human form. It is known that the variable domains of both heavy and light chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable domains can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

A number of humanized antibody molecules comprising an epitope-binding site derived from a non-human immunoglobulin have been described, including chimeric antibodies having rodent or modified rodent Variable Domain and their associated complementarity determining regions (CDRs) fused to human constant domains (see, for example, Winter et al. (1991) “Man-made Antibodies,” Nature 349:293-299; Lobuglio et al. (1989) “Mouse/Human Chimeric Monoclonal Antibody In Man: Kinetics And Immune Response,” Proc. Natl. Acad. Sci. (U.S.A.) 86:4220-4224 (1989), Shaw et al. (1987) “Characterization Of A Mouse/Human Chimeric Monoclonal Antibody (17-1A) To A Colon Cancer Tumor-Associated Antigen,” J. Immunol. 138:4534-4538, and Brown et al. (1987) “Tumor-Specific Genetically Engineered Murine/Human Chimeric Monoclonal Antibody,” Cancer Res. 47:3577-3583). Other references describe rodent CDRs grafted into a human supporting framework region (FR) prior to fusion with an appropriate human antibody Constant Domain (see, for example, Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; and Jones et al. (1986) “Replacing The Complementarity-Determining Regions In A Human Antibody With Those From A Mouse,” Nature 321:522-525). Another reference describes rodent CDRs supported by recombinantly veneered rodent framework regions. See, for example, European Patent Publication No. 519,596. These “humanized” molecules are designed to minimize unwanted immunological response towards rodent anti-human antibody molecules, which limits the duration and effectiveness of therapeutic applications of those moieties in human recipients. Other methods of humanizing antibodies that may also be utilized are disclosed by Daugherty et al. (1991) “Polymerase Chain Reaction Facilitates The Cloning, CDR-Grafting, And Rapid Expression Of A Murine Monoclonal Antibody Directed Against The CD18 Component Of Leukocyte Integrins,” Nucl. Acids Res. 19:2471-2476 and in U.S. Pat. Nos. 6,180,377; 6,054,297; 5,997,867; and 5,866,692.

### II. Fcγ Receptors (FcγRs)

The CH2 and CH3 Domains of the two heavy chains interact to form an “Fc Region,” which is a domain that is recognized by cellular Fc Receptors, including but not limited to Fc gamma Receptors (FcγRs). As used herein, the term “Fc Region” is used to define a C-terminal region of an IgG heavy chain. An Fc Region is said to be of a particular IgG isotype, class or subclass if its amino acid sequence is most homologous to that isotype relative to other IgG isotypes. In addition to their known uses in diagnostics, antibodies have been shown to be useful as therapeutic agents.

The amino acid sequence of the CH2-CH3 Domain of an exemplary human IgG1 is (SEQ ID NO:1):

```
231 240 250 260 270 280
APELLGGPSV FLFPPKPKDT LMISRTPEVT CVVVDVSHED PEVKFNWYVD
                  290 300 310 320 330
GVEVHNAKTK PREEQYNSTY RVVSVLTVLH QDWLNGKEYK CKVSNKALPA
                  340 350 360 370 380
PIEKTISKAK GQPREPQVYT LPPSREEMTK NQVSLTCLVK GFYPSDIAVE
                  390 400 410 420 430
WESNGQPENN YKTTPPVLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG2 is (SEQ ID NO:2):

```
231 240 250 260 270 280
APPVA-GPSV FLFPPKPKDT LMISRTPEVT CVVVDVSHED PEVQFNWYVD
                  290 300 310 320 330
GVEVHNAKTK PREEQFNSTF RVVSVLTVVH QDWLNGKEYK CKVSNKGLPA
                  340 350 360 370 380
PIEKTISKTK GQPREPQVYT LPPSREEMTK NQVSLTCLVK GFYPSDISVE
                  390 400 410 420 430
WESNGQPENN YKTTPPMLDS DGSFFLYSKL TVDKSRWQQG NVFSCSVMHE
                  440 447
ALHNHYTQKS LSLSPGX
```

as numbered by the EU index as set forth in Kabat, wherein X is a lysine (K) or is absent.

The amino acid sequence of the CH2-CH3 Domain of an exemplary human IgG3 is (SEQ ID NO:3):

```
231 240 250 260 270 280
APELLGGPSV FLFPPKPKDT LMISRTPEVT CVVVDVSHED PEVQFKWYVD
                  290 300 310 320 330
GVEVHNAKTK PREEQYNSTF RVVSVLTVLH QDWLNGKEYK CKVSNKALPA
                  340 350 360 370 380
PIEKTISKTK GQPREPQVYT LPPSREEMTK NQVSLTCLVK GFYPSDIAVE
                  390 400 410 420 430
WESSGQPENN YNTTPPMLDS DGSFFLYSKL TVDKSRWQQG NIFSCSVMHE
                  440 447
ALHNRFTQKS LSLSPGX
```

as numbered by the EU index as set forth in Kabat, wherein X is a lysine (K) or is absent.

The amino acid sequence of the CH2-CH3 Domain of an exemplary human IgG4 is (SEQ ID NO:4):

```
231 240 250 260 270 280
APEFLGGPSV FLFPPKPKDT LMISRTPEVT CVVVDVSQED PEVQFNWYVD
                  290 300 310 320 330
GVEVHNAKTK PREEQFNSTY RVVSVLTVLH QDWLNGKEYK CKVSNKGLPS
                  340 350 360 370 380
SIEKTISKAK GQPREPQVYT LPPSQEEMTK NQVSLTCLVK GFYPSDIAVE
                  390 400 410 420 430
WESNGQPENN YKTTPPVLDS DGSFFLYSRL TVDKSRWQEG NVFSCSVMHE
                  440 447
ALHNHYTQKS LSLSLGX
```

as numbered by the EU index as set forth in Kabat, wherein X is a lysine (K) or is absent.

Throughout the present specification, the numbering of the residues in the constant region of an IgG heavy chain is that of the EU index as in Kabat et al., SEQUENCES OF PROTEINS OF IMMUNOLOGICAL INTEREST, 5^{th} Ed. Public Health Service, NH1, MD (1991) (“Kabat”), expressly incorporated herein by reference. The term “EU index as in Kabat” refers to the numbering of the constant domains of human IgG1 EU antibody. Amino acids from the Variable Domains of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical Structures For The Hypervariable Regions Of Immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain.

Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the antibodies of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the ROR1-binding molecules of the invention. Specifically encompassed by the instant invention are ROR1-binding molecules lacking the C-terminal residue of the CH3 Domain. Also specifically encompassed by the instant invention are such constructs comprising the C-terminal lysine residue of the CH3 Domain.

As stated above, the Fc Region of natural IgG antibodies is capable of binding to cellular Fc gamma Receptors (FcγRs). Such binding results in the transduction of activating or inhibitory signals to the immune system. The ability of such binding to result in diametrically opposing functions reflects structural differences among the different FcγRs, and in particular reflects whether the bound FcγR possesses an immunoreceptor tyrosine-based activation motif (“ITAM”) or an immunoreceptor tyrosine-based inhibitory motif (“ITIM”). The recruitment of different cytoplasmic enzymes to these structures dictates the outcome of the FcγR-mediated cellular responses. ITAM-containing FcγRs include FcγRI, FcγRIIA, FcγRIIIA, and activate the immune system when bound to Fc

Regions (e.g., aggregated Fc Regions present in an immune complex). FcγRIIB is the only currently known natural ITIM-containing FcγR; it acts to dampen or inhibit the immune system when bound to aggregated Fc Regions. Human neutrophils express the FcγRIIA gene. FcγRIIA clustering via immune complexes or specific antibody cross-linking serves to aggregate ITAMs with receptor-associated kinases which facilitate ITAM phosphorylation. ITAM phosphorylation serves as a docking site for Syk kinase, the activation of which results in the activation of downstream substrates (e.g., PI₃K). Cellular activation leads to release of pro-inflammatory mediators. The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently the molecular basis of this inhibition was established. When co-ligated along with an activating FcγR, the ITIM in FcγRIIB becomes phosphorylated and attracts the SH2 domain of the inositol polyphosphate 5′-phosphatase (SHIP), which hydrolyzes phosphoinositol messengers released as a consequence of ITAM-containing FcγR-mediated tyrosine kinase activation, consequently preventing the influx of intracellular Ca++. Thus cross-linking of FcγRIIB dampens the activating response to FcγR ligation and inhibits cellular responsiveness. B-cell activation, B-cell proliferation and antibody secretion is thus aborted.

### III. Bispecific Antibodies, Multispecific Diabodies and DART® Diabodies

The ability of an antibody to bind an epitope of an antigen depends upon the presence and amino acid sequence of the antibody's VL and VH Domains. Interaction of an antibody's Light Chain and Heavy Chain and, in particular, interaction of its VL and VH Domains forms one of the two epitope-binding sites of a natural antibody, such as an IgG. Natural antibodies are capable of binding to only one epitope species (i.e., they are monospecific), although they can bind multiple copies of that species (i.e., exhibiting bivalency or multivalency).

The binding domains of the present invention, bind to epitopes in an “immunospecific” manner. As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “immunospecific binding” does not necessarily require (although it can include) exclusive binding. Generally, but not necessarily, reference to binding means “immunospecific” binding. Two molecules are said to be capable of binding to one another in a “physiospecific” manner, if such binding exhibits the specificity with which receptors bind to their respective ligands.

The functionality of antibodies can be enhanced by generating multispecific antibody-based molecules that can simultaneously bind two separate and distinct antigens (or different epitopes of the same antigen) and/or by generating antibody-based molecule having higher valency (i.e., more than two binding sites) for the same epitope and/or antigen.

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (WO 2005/070966, WO 2006/107786A WO 2006/107617A, WO 2007/046893). PCT Publications Nos. WO 2013/174873, WO 2011/133886 and WO 2010/136172 disclose a trispecific antibody in which the CL and CH1 Domains are switched from their respective natural positions and the VL and VH Domains have been diversified (WO 2008/027236; WO 2010/108127) to allow them to bind to more than one antigen. PCT Publications Nos. WO 2013/163427 and WO 2013/119903 disclose modifying the CH2 Domain to contain a fusion protein adduct comprising a binding domain. PCT Publications Nos. WO 2010/028797, WO02010028796 and WO 2010/028795 disclose recombinant antibodies whose Fc Regions have been replaced with additional VL and VH Domains, so as to form trivalent binding molecules. PCT Publications Nos. WO 2003/025018 and WO2003012069 disclose recombinant diabodies whose individual chains contain scFv Domains. PCT Publication Nos. WO 2013/006544 discloses multivalent Fab molecules that are synthesized as a single polypeptide chain and then subjected to proteolysis to yield heterodimeric structures. PCT Publications Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another).

The art has additionally noted the capability to produce diabodies that differ from such natural antibodies in being capable of binding two or more different epitope species (i.e., exhibiting bispecificity or multispecificity in addition to bivalency or multivalency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388/WO 02/02781 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng. Des. Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The design of a diabody is based on the antibody derivative known as a single-chain Variable Domain fragment (scFv). Such molecules are made by linking Light and/or Heavy Chain Variable Domains by using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The provision of bispecific binding molecules (e.g., non-monospecific diabodies) provides a significant advantage over antibodies, including but not limited to, a “trans” binding capability sufficient to co-ligate and/or co-localize different cells that express different epitopes and/or a “cis” binding capability sufficient to co-ligate and/or co-localize different molecules expressed by the same cell. Bispecific binding molecules (e.g., non-monospecific diabodies) thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221-1225).

The ability to produce bispecific diabodies has led to their use (in “trans”) to co-ligate two cells together, for example, by co-ligating receptors that are present on the surface of different cells (e.g., cross-linking cytotoxic T-cells to tumor cells) (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305; Marvin et al. (2005) “Recombinant Approaches To IgG-Like Bispecific Antibodies,” Acta Pharmacol. Sin. 26:649-658). Alternatively (or additionally), bispecific (or tri- or multispecific) diabodies can be used (in “cis”) to co-ligate molecules, such as receptors, etc., that are present on the surface of the same cell. Co-ligation of different cells and/or receptors is useful to modulate effector functions and/or immune cell signaling. Multispecific molecules (e.g., bispecific diabodies) comprising epitope-binding sites may be directed to a surface determinant of any immune cell such as CD2, CD3, CD8, CD16, T-Cell Receptor (TCR), NKG2D, etc., which are expressed on T lymphocytes, Natural Killer (NK) cells, Antigen-Presenting Cells or other mononuclear cells. In particular, epitope-binding sites directed to a cell surface receptor that is present on immune effector cells, are useful in the generation of multispecific binding molecules capable of mediating redirected cell killing.

However, the above advantages come at a salient cost. The formation of such non-monospecific diabodies requires the successful assembly of two or more distinct and different polypeptides (i.e., such formation requires that the diabodies be formed through the heterodimerization of different polypeptide chain species). This fact is in contrast to monospecific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20): 19665-19672).

However, the art has recognized that bispecific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies, termed DART® (Dual-Affinity Re-Targeting Reagents) diabodies; see, e.g., United States Patent Publication Nos. 2013-0295121; 2010-0174053 and 2009-0060910; European Patent Publication No. EP 2714079; EP 2601216; EP 2376109; EP 2158221 and PCT Publication Nos. WO 2012/162068; WO 2012/018687; WO 2010/080538; and Sloan, D. D. et al. (2015) “Targeting HIV Reservoir in Infected CD4 T Cells by Dual-Affinity Re-targeting Molecules(DARTs) that Bind HIV Envelope and Recruit Cytotoxic T Cells,” PLoS Pathog. 11(11):e1005233. doi: 10.1371/journal.ppat.1005233; Al Hussaini, M. et al. (2015) “Targeting CD123 In AML Using A T-Cell Directed Dual-Affinity Re-Targeting (DART®) Platform,” Blood pii: blood-2014-05-575704; Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond one or more pairs of such polypeptide chains to one another. For example, the addition of a cysteine residue to the C-terminus of such constructs has been shown to allow disulfide bonding between the involved polypeptide chains, stabilizing the resulting diabody without interfering with the diabody' s binding characteristics.

Many variations of such molecules have been described (see, e.g., United States Patent Publication Nos. 2015/0175697; 2014/0255407; 2014/0099318; 2013/0295121; 2010/0174053; 2009/0060910; 2007-0004909; European Patent Publication Nos. EP 2714079; EP 2601216; EP 2376109; EP 2158221; EP 1868650; and PCT Publication Nos. WO 2012/162068; WO 2012/018687; WO 2010/080538; WO 2006/113665), and are provided herein.

Alternative constructs are known in the art for applications where a tetravalent molecule is desirable but an Fc is not required including, but not limited to, tetravalent tandem antibodies, also referred to as “TandAbs” (see, e.g. United States Patent Publications Nos. 2005-0079170, 2007-0031436, 2010-0099853, 2011-020667 2013-0189263; European Patent Publication Nos. EP 1078004, EP 2371866, EP 2361936 and EP 1293514; PCT Publications Nos. WO 1999/057150, WO 2003/025018, and WO 2013/013700) which are formed by the homo-dimerization of two identical polypeptide chains, each possessing a VH1, VL2, VH2, and VL2 Domain.

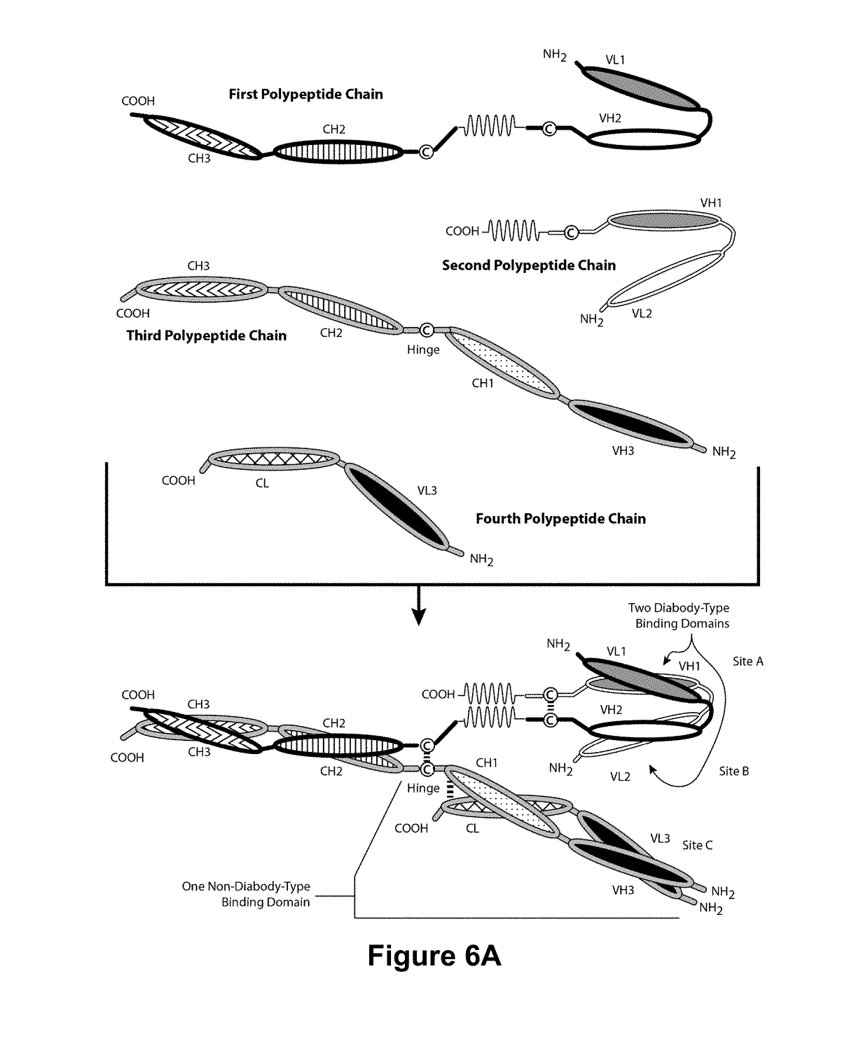

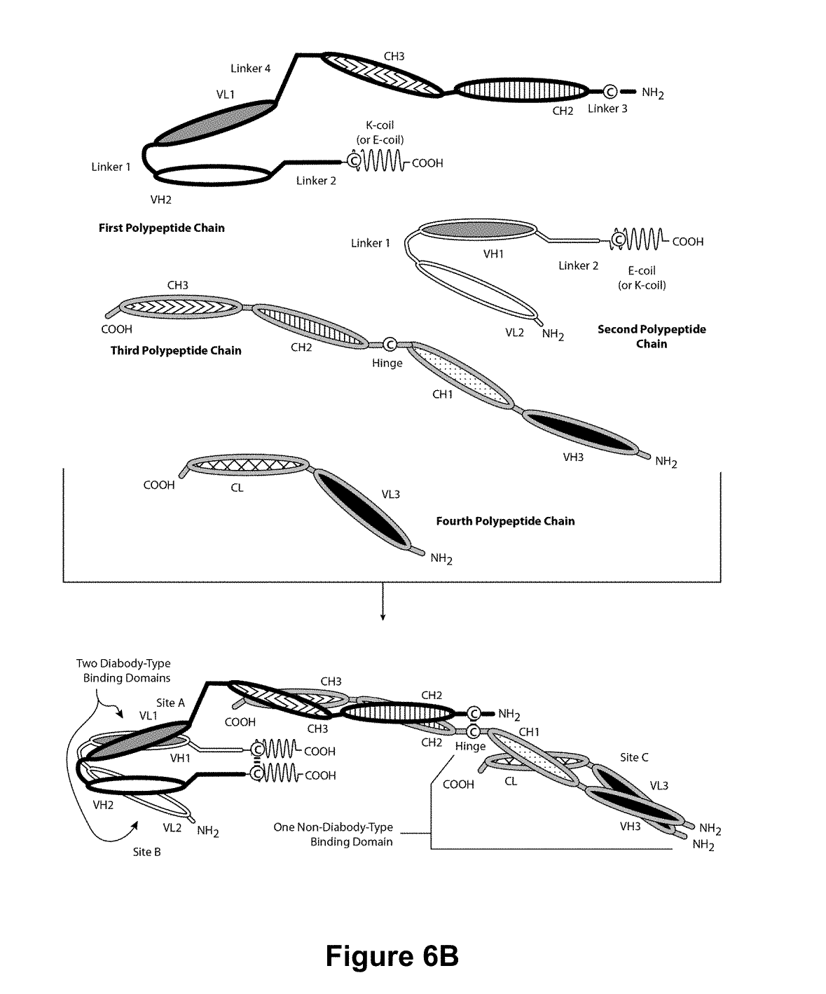

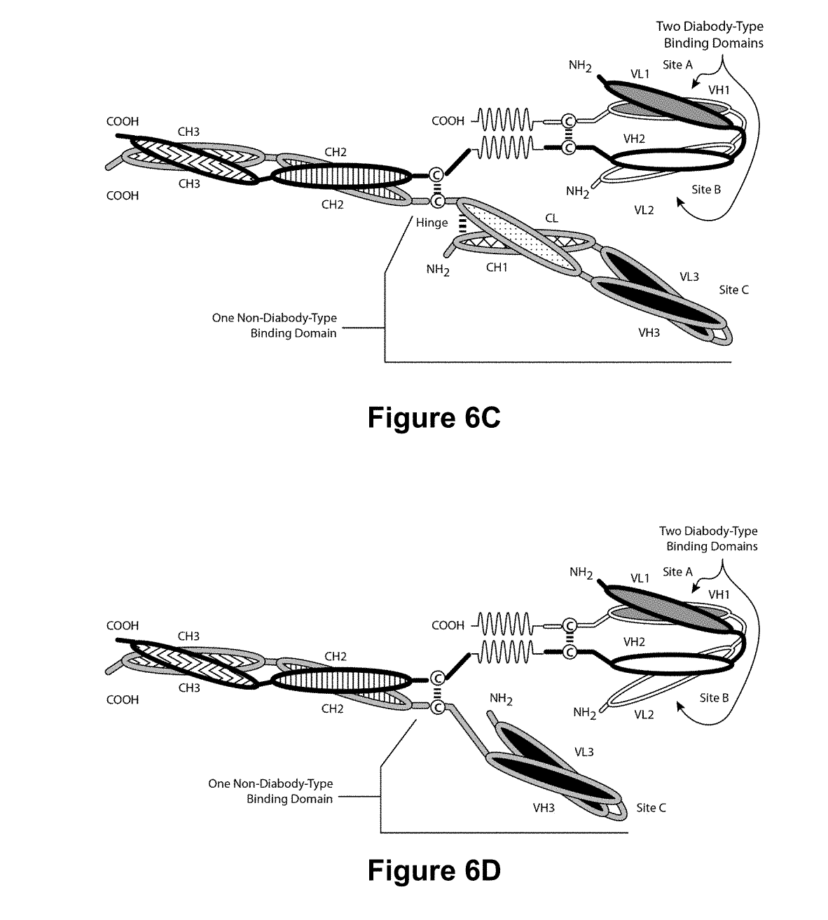

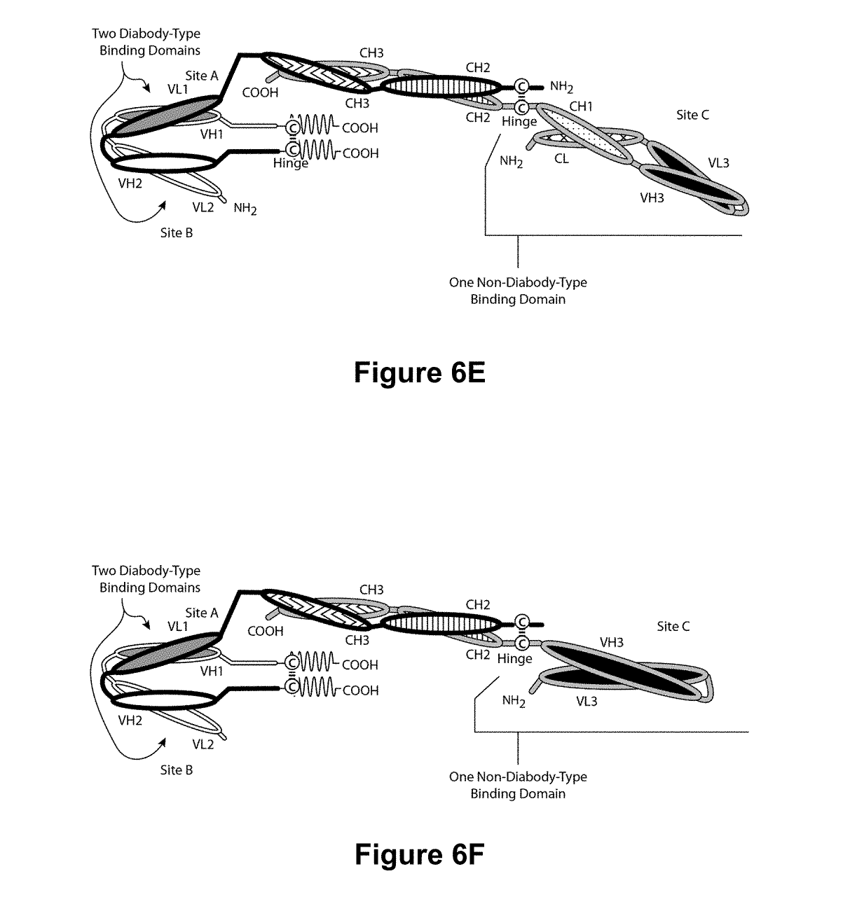

Recently, trivalent structures incorporating two diabody-type binding domains and one non-diabody-type domain and an Fc Region have been described (see, e.g., PCT Publication Nos. WO 2015/184207 and WO 2015/184203). Such trivalent binding molecules may be utilized to generate monospecific, bispecific or trispecific molecules. The ability to bind three different epitopes provides enhanced capabilities. FIGS. 6A-6F provide schematics of such trivalent binding molecules comprising 3 or 4 polypeptide chains.

### IV. Optimized Anti-ROR1 Variable Domains

The preferred optimized ROR1-binding molecules of the present invention include antibodies, diabodies, BiTEs, trivalent binding etc. capable of binding to a continuous or discontinuous (e.g., conformational) epitope of human ROR1. The optimized ROR1-binding molecules of the present invention will preferably also exhibit the ability to bind to the ROR1 molecules of one or more non-human species, especially, a non-human primate species (e.g., cynomolgus monkey, chimpanzee, macaque, etc.). A representative long isoform of a human ROR1 polypeptide (NCBI Sequence NP_005003.2, including a 29-amino acid residue signal sequence, shown underlined) (SEQ ID NO:5) is:

MHRPRRRGTR PPLLALLAAL LLAARGAAAQ ETELSVSAEL
VPTSSWNISS ELNKDSYLTL DEPMNNITTS LGQTAELHCK
VSGNPPPTIR WFKNDAPVVQ EPRRLSFRST IYGSRLRIRN
LDTTDTGYFQ CVATNGKEVV SSTGVLFVKF GPPPTASPGY
SDEYEEDGFC QPYRGIACAR FIGNRTVYME SLHMQGEIEN
QITAAFTMIG TSSHLSDKCS QFAIPSLCHY AFPYCDETSS
VPKPRDLCRD ECEILENVLC QTEYIFARSN PMILMRLKLP
NCEDLPQPES PEAANCIRIG IPMADPINKN HKCYNSTGVD
YRGTVSVTKS GRQCQPWNSQ YPHTHTFTAL RFPELNGGHS
YCRNPGNQKE APWCFTLDEN FKSDLCDIPA CDSKDSKEKN
KMEILYILVP SVAIPLAIAL LFFFICVCRN NQKSSSAPVQ
RQPKHVRGQN VEMSMLNAYK PKSKAKELPL SAVRFMEELG
ECAFGKIYKG HLYLPGMDHA QLVAIKTLKD YNNPQQWTEF
QQEASLMAEL HHPNIVCLLG AVTQEQPVCM LFEYINQGDL
HEFLIMRSPH SDVGCSSDED GTVKSSLDHG DFLHIAIQIA
AGMEYLSSHF FVHKDLAARN ILIGEQLHVK ISDLGLSREI
YSADYYRVQS KSLLPIRWMP PEAIMYGKFS SDSDIWSFGV
VLWEIFSFGL QPYYGFSNQE VIEMVRKRQL LPCSEDCPPR
MYSLMTECWN EIPSRRPRFK DIHVRLRSWE GLSSHTSSTT
PSGGNATTQT TSLSASPVSN LSNPRYPNYM FPSQGITPQG
QIAGFIGPPI PQNQRFIPIN GYPIPPGYAA FPAAHYQPTG
PPRVIQHCPP PKSRSPSSAS GSTSTGHVTS LPSSGSNQEA
NIPLLPHMSI PNHPGGMGIT VFGNKSQKPY KIDSKQASLL
GDANIHGHTE SMISAEL

Of the 937 amino acid residues of ROR1 (SEQ ID NO:5), residues 1-29 are a signal sequence, residues 30-406 are the Extracellular Domain, residues 407-427 are the Transmembrane Domain, and residues 428-937 are the Cytoplasmic Domain. Several isoforms and natural variants are known.

The present invention particularly encompasses ROR1-binding molecules (e.g., antibodies, diabodies, trivalent binding molecules, etc.,) comprising optimized anti-ROR1 Variable Domains (i.e., VL and/or VH Domains) that immunospecifically bind to an epitope of a human ROR1 polypeptide. As used herein such ROR1 Variable Domains are referred to as “anti-ROR1-VL” and “anti-ROR1-VH,” respectively.

The ROR1-binding molecules of the present invention particularly comprise molecules having optimized anti-ROR1-VL Domains and/or anti-ROR1-VH Domains) that immunospecifically bind to an epitope of a human ROR1 polypeptide, especially a human ROR1 polypeptide that comprises residues 30-406 of SEQ ID NO:5. Preferably, such optimized ROR1-binding molecules exhibit enhanced binding affinity for human ROR1, and/or are deimmunized to reduce the immunogenicity of such molecules, both as compared to a ROR1-binding molecule comprising the non-optimized parental anti-ROR1-VL and anti-ROR1-VH Domains. More preferably, the present invention pertains to optimized ROR1-binding molecules that exhibit enhanced binding affinity for ROR1 and reduced immunogenicity.

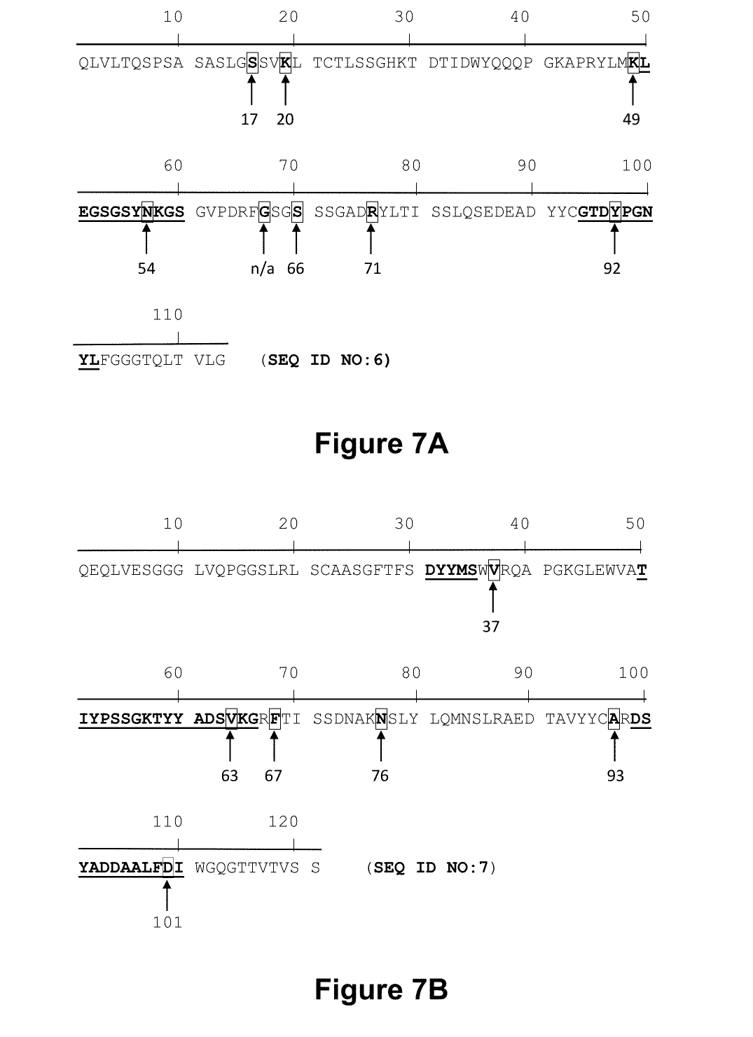

The amino acid sequence of the parental anti-ROR1-VL Domain (SEQ ID NO:6) is provided below and in FIG. 7A, the CDR_{L} residues are shown underlined.

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLG

The amino acid sequence of the parental anti-ROR1-VH (SEQ ID NO:7) is provided below and in FIG. 7B, the CDRH residues are shown underlined.

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCARDS YADDAALFDI WGQGTTVTVS S

In certain embodiments ROR1-binding molecules (e.g., scFvs, antibodies, bispecific diabodies, etc.) comprising the optimized anti-ROR1-VL and/or VH Domains of the invention are characterized by any one, two, three, four, five, six, seven, eight or nine of the following criteria:
(1) the ability to immunospecifically bind human ROR1 as endogenously expressed on the surface of a cancer cell;
(2) the ability to immunospecifically bind human ROR1 with enhanced binding affinity relative to a ROR1-binding molecule comprising the parental anti-ROR1-VL and anti-ROR1-VH Domains;
(3) the ability to immunospecifically bind human ROR1 with a lower monovalent equilibrium binding constant (K_{D}) than that of a ROR1-binding molecule comprising the parental anti-ROR1-VL and anti-ROR1-VH Domains;
(4) the ability to immunospecifically bind human ROR1 with a monovalent equilibrium binding constant (K_{D}) at least two-fold lower than that of a ROR1-binding molecule comprising the parental anti-ROR1-VL and anti-ROR1-VH Domains;
(5) the ability to immunospecifically bind human ROR1 with a higher monovalent rate of association (kₐ) than that of a ROR1-binding molecule comprising the parental anti-ROR1-VL and anti-ROR1-VH Domains;
(6) the ability to immunospecifically bind human ROR1 with a lower monovalent rate of dissociation (kₐ) than that of a ROR1-binding molecule comprising the parental anti-ROR1-VL and anti-ROR1-VH Domains;
(7) the ability to immunospecifically bind non-human primate ROR1 (e.g., ROR1 of cynomolgus monkey);
(8) reduced immunogenicity relative to the immunogenicity of ROR1-binding molecule comprising the parental anti-ROR1-VL and anti-ROR1-VH Domains; and/or
(9) enhanced ability to mediate redirected cell killing relative to that (if any) of a ROR1-binding molecule comprising the parental anti-ROR1-VL and anti-ROR1-VH Domains.

As described elsewhere herein, the binding constants of a ROR1-binding molecule may be determined using surface plasmon resonance e.g., via a BIACORE® analysis. Surface plasmon resonance data may be fitted to a 1:1 Langmuir binding model (simultaneous ka kd) and an equilibrium binding constant K_{D} calculated from the ratio of rate constants kd/ka. Such binding constants may be determined for a monovalent ROR1-binding molecule (i.e., a molecule comprising a single ROR1 epitope-binding site), a bivalent ROR1-binding molecule (i.e., a molecule comprising two ROR1 epitope-binding sites), or ROR1-binding molecules having higher valency (e.g., a molecule comprising three, four, or more ROR1 epitope-binding sites).

As used herein the term “redirected cell killing” refers to the ability of a molecule to mediate the killing of a target cell (e.g., cancer cell) by localizing an immune effector cell (e.g., T-cell, NK cell, etc.) to the location of the target cell by binding epitopes present on the surfaces of such effector and target cells, resulting in the killing of the target cell. The ability of a ROR1-binding molecule (e.g., a bispecific ROR1×CD3-binding molecule) to mediate redirected cell killing activity may be determined using a cytotoxic T lymphocyte (CTL) assay. Such assays are well known in the art and preferred assays are described below.

The ROR1-binding molecules of the present invention comprise an optimized anti-ROR1-VL and/or anti-ROR1-VH Domain. In preferred embodiments the ROR1-binding molecules comprise an optimized anti-ROR1-VL Domain or an optimized anti-ROR1-VH Domain. In more preferred embodiments, the ROR1-binding molecules of the invention comprise an optimized anti-ROR1-VL Domain and an optimized anti-ROR1-VH Domain.

The amino acid sequences of preferred optimized anti-ROR1-VL Domains of the present invention are variants of SEQ ID NO:6 and are represented by SEQ ID NO:8 (CDR_{L} residues are shown underlined):

QLVLTQSPSA SASLGX₁SVX₂L TCTLSSGHKT DTIDWYQQQP
GKAPRYLMX₃L EGSGSYNKGS GVPDRFX₄SGX₅ SSGADX₆YLTI
SSLQSEDEAD YYCGTDX₇PGN YLFGGGTQLT VLG

wherein: X₁, X₂, X₃, X₄, X₅, X₆, and X₇ are independently selected, and wherein: X₁ is S or G, X₂ is K, I, or N, X₃ is K or N, X₄ is G or is absent, X₅ is S or I, X₆ is R or W, and X₇ is Y or N.

In a preferred embodiment, the ROR1-binding molecules of the invention comprise an optimized anti-ROR1-VL Domain having the amino acid sequence of SEQ ID NO:8, wherein X₆ is W.

In a further embodiment, the optimized ROR1-binding molecules of the invention comprise an optimized anti-ROR1-VL Domain having the amino acid sequence of SEQ ID NO:8, wherein X₆ is W and wherein:
(a) X₁ is S or G, X₂ is K, I or N, X₃ is K or N, X₄ is G or is absent, X₅ is S or I, X₇ is Y or N;
(b) X₁ is S, X₂ is K, X₃ is K, X₄ is G or is absent, X₅ is S, and X₇ is N;
(c) X₁ is S, X₂ is K, X₃ is K, X₄ is G or is absent, X₅ is I, and X₇ is Y;
(d) X₁ is S, X₂ is K, X₃ is K, X₄ is G or is absent, X₅ is I, and X₇ is N; or
(e) X₁ is S, X₂ is K, X₃ is K, X₄ is G or is absent, X₅ is S, and X₇ is Y.

The amino acid sequences of preferred optimized anti-ROR1-VH Domains of the present invention are variants of SEQ ID NO:7 and are represented by SEQ ID NO:9 (CDR_{H} residues are shown underlined):

QEQLVESGGG LVQPGGSLRL SCAASGFTFS DYYMSWX₁RQA
PGKGLEWVAT IYPSSGKTYY ADSX₂KGRX₃TI SSDNAKX₄SLY
LQMNSLRAED TAVYYCX₅RDS YADDAALFDI WGQGTTVTVS S

wherein: X₁, X₂, X₃, X₄, and X₅, are independently selected, and
wherein: X₁ is V or I, X₂ is V or A, X₃ is F or L, X₄ is N, D, or Y, and X₅ is A or T.

The invention particularly provides such an optimized ROR1-binding wherein the Variable Heavy Chain Domain has the amino acid sequence of SEQ ID NO:9, wherein:

(a) X₁ is V or I, X₂ is V or A, X₃ is L, X₄ is N, D, or Y, and X₅ is A or T;

(b) X₁ is V or I, X₂ is V or A, X₃ is F or L, X₄ is D or Y, and X₅ is A or T;

(c) X₁ is V or I, X₂ is V or A, X₃ is F or L, X₄ is N, D, or Y, and X₅ is T;

(d) X₁ is V or I, X₂ is V or A, X₃ is L, X₄ is N, and X₅ is A;

(e) X₁ is V or I, X₂ is V or A, X₃ is F, X₄ is D, and X₅ is A;

(f) X₁ is V or I, X₂ is V or A, X₃ is F, X₄ is N, and X₅ is T;

(g) X₁ is V or I, X₂ is V or A, X₃ is L, X₄ is D, and X₅ is T;

(h) X₁ is I, X₂ is A, X₃ is F or L, X₄ is N, D or Y, and X₅ is A or T;

(i) X₁ is I, X₂ is A, X₃ is F, X₄ is N, and X₅ is A;

(j) X₁ is I, X₂ is A, X₃ is L, X₄ is N, and X₅ is A;

(k) X₁ is I, X₂ is A, X₃ is F, X₄ is D, and X₅ is A;

(l) X₁ is I, X₂ is A, X₃ is F, X₄ is N, and X₅ is T; or

(m) X₁ is I, X₂ is A, X₃ is L, X₄ is D, and X₅ is T.

In a preferred embodiment, the ROR1-binding molecules of the invention comprise an optimized anti-ROR1-VH Domain having the amino acid sequence of SEQ ID NO:9, wherein:

(a) X₁ is V or I, X₂ is V or A, X₃ is L, X₄ is N, D, or Y, and X₅ is A or T;

(b) X₁ is V or I, X₂ is V or A, X₃ is F or L, X₄ is D or Y, and X₅ is A or T;

(c) X₁ is V or I, X₂ is V or A, X₃ is F or L, X₄ is N, D, or Y, and X₅ is T;

(d) X₁ is V or I, X₂ is V or A, X₃ is L, X₄ is N, and X₅ is A;

(e) X₁ is V or I, X₂ is V or A, X₃ is F, X₄ is D, and X₅ is A;

(f) X₁ is V or I, X₂ is V or A, X₃ is F, X₄ is N, and X₅ is T; or

(g) X₁ is V or I, X₂ is V or A, X₃ is L, X₄ is D, and X₅ is T.

In a further preferred embodiment, the ROR1-binding molecules of the invention comprise an optimized anti-ROR1-VH Domain having the amino acid sequence of SEQ ID NO:9, wherein X₁ is I and X₂ is A, and wherein:

(a) X₃ is F or L, X₄ is N, D or Y, and X₅ is A or T;

(b) X₃ is F, X₄ is N, and X₅ is A;

(c) X₃ is L, X₄ is N, and X₅ is A;

(d) X₃ is F, X₄ is D, and X₅ is A;

(e) X₃ is F, X₄ is N, and X₅ is T; or

(f) X₃ is L, X₄ is D, and X₅ is T.

In particular, as provided herein ROR1-binding molecules comprising fourteen different variants of the parental anti-ROR1-VL Domain (SEQ ID NO:6) were constructed and studied. The variant anti-ROR1-VL Domains were designated “anti-ROR1-VL(1),” “anti-ROR1-VL(2),” “anti-ROR1-VL(3),” “anti-ROR1-VL(4),” “anti-ROR1-VL(5),” “anti-ROR1-VL(6),” “anti-ROR1-VL(7),” “anti-ROR1-VL(8),” “anti-ROR1-VL(9),” “anti-ROR1-VL(10),” “anti-ROR1-VL(11),” “anti-ROR1-VL(12),” “anti-ROR1-VL(13),” and “anti-ROR1-VL(14).” The amino acid sequences of these variant VL Domains are presented below:

The amino acid sequence of anti-ROR1-VL(1) (SEQ ID NO:10) is shown below (modified residues are shown underlined):

QLVLTQSPSA SASLGSSVKL TCTLSSGHKT DTIDWYQQQP
GKAPRYLMKL EGSGSYNKGS GVPDRF-SGS SSGADRYLTI
SSLQSEDEAD YYCGTDYPGN YLFGGGTQLT VLG

The amino acid sequence of anti-ROR1-VL(2) (SEQ ID NO:11) is shown below (modified residues are shown underlined):

QLVLTQSPSA SASLGSSVKL TCTLSSGHKT DTIDWYQQQP
GKAPRYLMKL EGSGSYNKGS GVPDRFGSGS SSGADWYLTI
SSLQSEDEAD YYCGTDYPGN YLFGGGTQLT VLG

The amino acid sequence of anti-ROR1-VL(3) (SEQ ID NO:12) is shown below (modified residues are shown underlined):

QLVLTQSPSA SASLGSSVKL TCTLSSGHKT DTIDWYQQQP
GKAPRYLMNL EGSGSYNKGS GVPDRFGSGS SSGADRYLTI
SSLQSEDEAD YYCGTDYPGN YLFGGGTQLT VLG

The amino acid sequence of anti-ROR1-VL(4) (SEQ ID NO:13) is shown below (modified residues are shown underlined):

QLVLTQSPSA SASLGGSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLG

The amino acid sequence of anti-ROR1-VL(5) (SEQ ID NO:14) is shown below (modified residues are shown underlined):

QLVLTQSPSA SASLGSSVKL TCTLSSGHKT DTIDWYQQQP
GKAPRYLMKL EGSGSYSKGS GVPDRFGSGS SSGADRYLTI
SSLQSEDEAD YYCGTDYPGN YLFGGGTQLT VLG

The amino acid sequence of anti-ROR1-VL(6) (SEQ ID NO:15) is shown below (modified residues are shown underlined):

QLVLTQSPSA SASLGSSVKL TCTLSSGHKT DTIDWYQQQP
GKAPRYLMKL EGSGSYNKGS GVPDRFGSGI SSGADRYLTI
SSLQSEDEAD YYCGTDYPGN YLFGGGTQLT VLG

The amino acid sequence of anti-ROR1-VL(7) (SEQ ID NO:16) is shown below (modified residues are shown underlined):

QLVLTQSPSA SASLGSSVIL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLG

The amino acid sequence of anti-ROR1-VL(8) (SEQ ID NO:17) is shown below (modified residues are shown underlined):

QLVLTQSPSA SASLGSSVNL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLG

The amino acid sequence of anti-ROR1-VL(9) (SEQ ID NO:18) is shown below (modified residues are shown underlined):

QLVLTQSPSA SASLGSSVKL TCTLSSGHKT DTIDWYQQQP
GKAPRYLMKL EGSGSYTKGS GVPDRFGSGS SSGADRYLTI
SSLQSEDEAD YYCGTDYPGN YLFGGGTQLT VLG

The amino acid sequence of anti-ROR1-VL(10) (SEQ ID NO:19) is shown below (modified residues are shown underlined):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDNPGN YLFGGGTQLT VLG

The amino acid sequence of anti-ROR1-VL(11) (SEQ ID NO:20) is shown below (modified residues are shown underlined):

QLVLTQSPSA SASLGSSVKL TCTLSSGHKT DTIDWYQQQP
GKAPRYLMKL EGSGSYNKGS GVPDRFGSGS SSGADWYLTI
SSLQSEDEAD YYCGTDNPGN YLFGGGTQLT VLG

The amino acid sequence of anti-ROR1-VL(12) (SEQ ID NO:21) is shown below (modified residues are shown underlined):

QLVLTQSPSA SASLGSSVKL TCTLSSGHKT DTIDWYQQQP
GKAPRYLMKL EGSGSYNKGS GVPDRFGSGI SSGADWYLTI
SSLQSEDEAD YYCGTDYPGN YLFGGGTQLT VLG

The amino acid sequence of anti-ROR1-VL(13) (SEQ ID NO:22) is shown below (modified residues are shown underlined):

QLVLTQSPSA SASLGSSVKL TCTLSSGHKT DTIDWYQQQP
GKAPRYLMKL EGSGSYNKGS GVPDRFGSGI SSGADWYLTI
SSLQSEDEAD YYCGTDNPGN YLFGGGTQLT VLG

The amino acid sequence of anti-ROR1-VL(14) (SEQ ID NO:23) is shown below (modified residues are shown underlined):

QLVLTQSPSA SASLGSSVKL TCTLSSGHKT DTIDWYQQQP
GKAPRYLMKL EGSGSYNKGS GVPDRF-SGS SSGADWYLTI
SSLQSEDEAD YYCGTDYPGN YLFGGGTQLT VLG

The particular modifications studied are summarized in Table 6, and the amino acid residues modified are boxed and indicated with an arrow in the amino acid sequence of anti-ROR1-VL presented in FIG. 7A (Kabat numbers are shown underneath). Although it can be seen that a number of amino acid residues have been substituted or deleted in these particular variants anti-ROR1-VL Domains, it is not necessary to modify all or most of these residues when engineering the optimized anti-ROR1-VL Domains of the invention. For the light chain variable region, it is preferable to modify the residue at Kabat position 71 (corresponding to residue 76 (X₆) of SEQ ID NO:6). In particular, the light chain may further comprise modifications at one or more of Kabat positions 66 and 92 (corresponding to residues 70 (X₅) and 97 (X₇) of SEQ ID NO:6). In addition, it will be noted that anti-ROR1-VL comprises an extra Glycine (G) residue between Kabat positions 63 and 64, accordingly, the light chain may further comprise a deletion of such extra amino acid residue (corresponding to residue 67 (X₄) of SEQ ID NO:6). In a preferred embodiment, an optimized anti-ROR1-VL Domain comprises a R71W substitution, and may optionally comprise: (1) a S661 substitution and/or (2) a Y92N substitution, and/or (3) a deletion of the G residue between 63 and 64, although as provided herein number of other modifications may be made. The present invention also encompasses minor variations of these sequences including, for example amino acid substitutions of the C-terminal and/or N-terminal amino acid residues which may be introduced to facilitate subcloning.

In various embodiments, the ROR1-binding molecules of the present invention comprise an optimized anti-ROR1-VL Domain, which VL Domain preferably comprises an amino acid sequence selected from the group consisting of: SEQ ID NO: 11, 19, 20, 21, 22, and 23. In a preferred embodiment, the ROR1-binding molecules of the present invention comprise an optimized anti-ROR1-VH Domain that comprises the amino acid sequence of SEQ ID NO: 11 or SEQ ID NO: 23.

In particular, as provided herein ROR1-binding molecules comprising eight different variants of the parental anti-ROR1-VH Domain (SEQ ID NO:7) were constructed and studied. The variant anti-ROR1-VH Domains were designated “anti-ROR1-VH(1),” “anti-ROR1-VH(2),” “anti-ROR1-VH(3),” “anti-ROR1-VH(4),” “anti-ROR1-VH(5),” “anti-ROR1-VH(6),” “anti-ROR1-VH(7),” and “anti-ROR1-VH(8).” An additional variant (designated “anti-ROR1-VH(9)”), which may be constructed is also provided. The amino acid sequences of these variant VH Domains are presented below:

The amino acid sequence of anti-ROR1-VH(1) (SEQ ID NO:24) is shown below (modified residues are shown underlined):

QEQLVESGGG LVQPGGSLRL SCAASGFTFS DYYMSWVRQA
PGKGLEWVAT IYPSSGKTYY ADSVKGRLTI SSDNAKNSLY
LQMNSLRAED TAVYYCARDS YADDAALFDI WGQGTTVTVS S

The amino acid sequence of anti-ROR1-VH(2) (SEQ ID NO:25) is shown below (modified residues are shown underlined):

QEQLVESGGG LVQPGGSLRL SCAASGFTFS DYYMSWVRQA
PGKGLEWVAT IYPSSGKTYY ADSVKGRFTI SSDNAKDSLY
LQMNSLRAED TAVYYCARDS YADDAALFDI WGQGTTVTVS S

The amino acid sequence of anti-ROR1-VH(3) (SEQ ID NO:26) is shown below (modified residues are shown underlined):

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCTRDS YADDAALFDI WGQGTTVTVS S

The amino acid sequence of anti-ROR1-VH(4) (SEQ ID NO:27) is shown below (modified residues are shown underlined):

QEQLVESGGG LVQPGGSLRL SCAASGFTFS DYYMSWVRQA
PGKGLEWVAT IYPSSGKTYY ADSVKGRFTI SSDNAKYSLY
LQMNSLRAED TAVYYCARDS YADDAALFDI WGQGTTVTVS S

The amino acid sequence of anti-ROR1-VH(5) (SEQ ID NO:28) is shown below (modified residues are shown underlined):

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCARDS YADDAALFAI WGQGTTVTVS
S

The amino acid sequence of anti-ROR1-VH(6) (SEQ ID NO:29) is shown below (modified residues are shown underlined):

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCARDS YADDAALFYI WGQGTTVTVS
S

The amino acid sequence of anti-ROR1-VH(7) (SEQ ID NO:30) is shown below (modified residues are shown underlined):

QEQLVESGGG LVQPGGSLRL SCAASGFTFS DYYMSWVRQA
PGKGLEWVAT IYPSSGKTYY ADSVKGRLTI SSDNAKDSLY
LQMNSLRAED TAVYYCTRDS YADDAALFDI WGQGTTVTVS
S

The amino acid sequence of anti-ROR1-VH(8) (SEQ ID NO:31) is shown below (modified residues are shown underlined):

QEQLVESGGG LVQPGGSLRL SCAASGFTFS DYYMSWIRQA
PGKGLEWVAT IYPSSGKTYY ADSAKGRLTI SSDNAKDSLY
LQMNSLRAED TAVYYCTRDS YADDAALFDI WGQGTTVTVS
S

The amino acid sequence of anti-ROR1-VH(9) (SEQ ID NO:32) is shown below (modified residues are shown underlined):

QEQLVESGGG LVQPGGSLRL SCAASGFTFS DYYMSWIRQA
PGKGLEWVAT IYPSSGKTYY ADSAKGRFTI SSDNAKNSLY
LQMNSLRAED TAVYYCARDS YADDAALFDI WGQGTTVTVS
S

The particular modifications studied are summarized in Table 6, and the amino acid residues modified are boxed and indicated with an arrow in the amino acid sequence of anti-ROR1-VH presented in FIG. 7B (Kabat numbers are shown underneath). Although it can be seen that a number of amino acid residues have been substituted or deleted in these particular optimized anti-ROR1-VH Domains, it is not necessary to modify all or most of these residues when engineering the optimized anti-ROR1-VH Domains the invention. For the heavy chain variable region, it is preferable to modify one or more residues at Kabat positions 67, 76, and 93 (corresponding to residues 68 (X₃), 77 (X₄), and 97 (X₅) of SEQ ID NO:9). In addition, or alternatively, the heavy chain may comprise modifications at one or more of Kabat positions 37 and 67 (corresponding to residues 37 (X₁) and 64 (X₂) of SEQ ID NO:9). In a preferred embodiment, an optimized anti-ROR1-VH Domain comprises: (1) a F67L substitution and/or (2) a N76D substitution, and/or (3) an A93T substitution, and/or (4) a V37I substitution, and/or (5) a V63A substitution, although as provided herein number of other modifications may be made. The present invention also encompasses minor variations of these sequences including, for example amino acid substitutions of the C-terminal and/or N-terminal amino acid residues which may be introduced to facilitate subcloning.

In various embodiments, the ROR1-binding molecules of the present invention comprise an optimized anti-ROR1-VH Domain, which VH Domain preferably comprises an amino acid sequence selected from the group consisting of: SEQ ID NO: 24, 25, 26, 27, 30, 31, and 32. In a preferred embodiment, the ROR1-binding molecules of the present invention comprise an optimized anti-ROR1-VH Domain that comprises the amino acid sequence of SEQ ID NO: 26, 30, 31, or 32.

In still other embodiments, the ROR1-binding molecules of the present invention comprise an optimized anti-ROR1-VL Domain, and also comprise an optimized anti-ROR1-VH Domain. The ROR1-binding molecules of the present invention may comprise any combination of the optimized anti-ROR1-VL and anti-ROR1-VH Domains described herein:

Anti-ROR1-VL(1) and Anti-ROR1-VH(1)
Anti-ROR1-VL(1) and Anti-ROR1-VH(2)
Anti-ROR1-VL(1) and Anti-ROR1-VH(3)
Anti-ROR1-VL(1) and Anti-ROR1-VH(4)
Anti-ROR1-VL(1) and Anti-ROR1-VH(5)
Anti-ROR1-VL(1) and Anti-ROR1-VH(6)
Anti-ROR1-VL(1) and Anti-ROR1-VH(7)
Anti-ROR1-VL(1) and Anti-ROR1-VH(8)
Anti-ROR1-VL(1) and Anti-ROR1-VH(9)
Anti-ROR1-VL(2) and Anti-ROR1-VH(1)
Anti-ROR1-VL(2) and Anti-ROR1-VH(2)
Anti-ROR1-VL(2) and Anti-ROR1-VH(3)
Anti-ROR1-VL(2) and Anti-ROR1-VH(4)
Anti-ROR1-VL(2) and Anti-ROR1-VH(5)
Anti-ROR1-VL(2) and Anti-ROR1-VH(6)
Anti-ROR1-VL(2) and Anti-ROR1-VH(7)
Anti-ROR1-VL(2) and Anti-ROR1-VH(8)
Anti-ROR1-VL(2) and Anti-ROR1-VH(9)
Anti-ROR1-VL(3) and Anti-ROR1-VH(1)
Anti-ROR1-VL(3) and Anti-ROR1-VH(2)
Anti-ROR1-VL(3) and Anti-ROR1-VH(3)
Anti-ROR1-VL(3) and Anti-ROR1-VH(4)
Anti-ROR1-VL(3) and Anti-ROR1-VH(5)
Anti-ROR1-VL(3) and Anti-ROR1-VH(6)
Anti-ROR1-VL(3) and Anti-ROR1-VH(7)
Anti-ROR1-VL(3) and Anti-ROR1-VH(8)
Anti-ROR1-VL(3) and Anti-ROR1-VH(9)
Anti-ROR1-VL(4) and Anti-ROR1-VH(1)
Anti-ROR1-VL(4) and Anti-ROR1-VH(2)
Anti-ROR1-VL(4) and Anti-ROR1-VH(3)
Anti-ROR1-VL(4) and Anti-ROR1-VH(4)
Anti-ROR1-VL(4) and Anti-ROR1-VH(5)
Anti-ROR1-VL(4) and Anti-ROR1-VH(6)
Anti-ROR1-VL(4) and Anti-ROR1-VH(7)
Anti-ROR1-VL(4) and Anti-ROR1-VH(8)
Anti-ROR1-VL(4) and Anti-ROR1-VH(9)
Anti-ROR1-VL(5) and Anti-ROR1-VH(1)
Anti-ROR1-VL(5) and Anti-ROR1-VH(2)
Anti-ROR1-VL(5) and Anti-ROR1-VH(3)
Anti-ROR1-VL(5) and Anti-ROR1-VH(4)
Anti-ROR1-VL(5) and Anti-ROR1-VH(5)
Anti-ROR1-VL(5) and Anti-ROR1-VH(6)
Anti-ROR1-VL(5) and Anti-ROR1-VH(7)
Anti-ROR1-VL(5) and Anti-ROR1-VH(8)
Anti-ROR1-VL(5) and Anti-ROR1-VH(9)
Anti-ROR1-VL(6) and Anti-ROR1-VH(1)
Anti-ROR1-VL(6) and Anti-ROR1-VH(2)
Anti-ROR1-VL(6) and Anti-ROR1-VH(3)
Anti-ROR1-VL(6) and Anti-ROR1-VH(4)
Anti-ROR1-VL(6) and Anti-ROR1-VH(5)
Anti-ROR1-VL(6) and Anti-ROR1-VH(6)
Anti-ROR1-VL(6) and Anti-ROR1-VH(7)
Anti-ROR1-VL(6) and Anti-ROR1-VH(8)
Anti-ROR1-VL(6) and Anti-ROR1-VH(9)
Anti-ROR1-VL(7) and Anti-ROR1-VH(1)
Anti-ROR1-VL(7) and Anti-ROR1-VH(2)
Anti-ROR1-VL(7) and Anti-ROR1-VH(3)
Anti-ROR1-VL(7) and Anti-ROR1-VH(4)
Anti-ROR1-VL(7) and Anti-ROR1-VH(5)
Anti-ROR1-VL(7) and Anti-ROR1-VH(6)
Anti-ROR1-VL(7) and Anti-ROR1-VH(7)
Anti-ROR1-VL(7) and Anti-ROR1-VH(8)
Anti-ROR1-VL(7) and Anti-ROR1-VH(9)
Anti-ROR1-VL(8) and Anti-ROR1-VH(1)
Anti-ROR1-VL(8) and Anti-ROR1-VH(2)
Anti-ROR1-VL(8) and Anti-ROR1-VH(3)
Anti-ROR1-VL(8) and Anti-ROR1-VH(4)
Anti-ROR1-VL(8) and Anti-ROR1-VH(5)
Anti-ROR1-VL(8) and Anti-ROR1-VH(6)
Anti-ROR1-VL(8) and Anti-ROR1-VH(7)
Anti-ROR1-VL(8) and Anti-ROR1-VH(8)
Anti-ROR1-VL(8) and Anti-ROR1-VH(9)
Anti-ROR1-VL(9) and Anti-ROR1-VH(1)
Anti-ROR1-VL(9) and Anti-ROR1-VH(2)
Anti-ROR1-VL(9) and Anti-ROR1-VH(3)
Anti-ROR1-VL(9) and Anti-ROR1-VH(4)
Anti-ROR1-VL(9) and Anti-ROR1-VH(5)
Anti-ROR1-VL(9) and Anti-ROR1-VH(6)
Anti-ROR1-VL(9) and Anti-ROR1-VH(7)
Anti-ROR1-VL(9) and Anti-ROR1-VH(8)
Anti-ROR1-VL(9) and Anti-ROR1-VH(9)
Anti-ROR1-VL(10) and Anti-ROR1-VH(1)
Anti-ROR1-VL(10) and Anti-ROR1-VH(2)
Anti-ROR1-VL(10) and Anti-ROR1-VH(3)
Anti-ROR1-VL(10) and Anti-ROR1-VH(4)
Anti-ROR1-VL(10) and Anti-ROR1-VH(5)
Anti-ROR1-VL(10) and Anti-ROR1-VH(6)
Anti-ROR1-VL(10) and Anti-ROR1-VH(7)
Anti-ROR1-VL(10) and Anti-ROR1-VH(8)
Anti-ROR1-VL(10) and Anti-ROR1-VH(9)
Anti-ROR1-VL(11) and Anti-ROR1-VH(1)
Anti-ROR1-VL(11) and Anti-ROR1-VH(2)
Anti-ROR1-VL(11) and Anti-ROR1-VH(3)
Anti-ROR1-VL(11) and Anti-ROR1-VH(4)
Anti-ROR1-VL(11) and Anti-ROR1-VH(5)
Anti-ROR1-VL(11) and Anti-ROR1-VH(6)
Anti-ROR1-VL(11) and Anti-ROR1-VH(7)
Anti-ROR1-VL(11) and Anti-ROR1-VH(8)
Anti-ROR1-VL(11) and Anti-ROR1-VH(9)
Anti-ROR1-VL(12) and Anti-ROR1-VH(1)
Anti-ROR1-VL(12) and Anti-ROR1-VH(2)
Anti-ROR1-VL(12) and Anti-ROR1-VH(3)
Anti-ROR1-VL(12) and Anti-ROR1-VH(4)
Anti-ROR1-VL(12) and Anti-ROR1-VH(5)
Anti-ROR1-VL(12) and Anti-ROR1-VH(6)
Anti-ROR1-VL(12) and Anti-ROR1-VH(7)
Anti-ROR1-VL(12) and Anti-ROR1-VH(8)
Anti-ROR1-VL(12) and Anti-ROR1-VH(9)
Anti-ROR1-VL(13) and Anti-ROR1-VH(1)
Anti-ROR1-VL(13) and Anti-ROR1-VH(2)
Anti-ROR1-VL(13) and Anti-ROR1-VH(3)
Anti-ROR1-VL(13) and Anti-ROR1-VH(4)
Anti-ROR1-VL(13) and Anti-ROR1-VH(5)
Anti-ROR1-VL(13) and Anti-ROR1-VH(6)
Anti-ROR1-VL(13) and Anti-ROR1-VH(7)
Anti-ROR1-VL(13) and Anti-ROR1-VH(8)
Anti-ROR1-VL(13) and Anti-ROR1-VH(9)
Anti-ROR1-VL(14) and Anti-ROR1-VH(1)
Anti-ROR1-VL(14) and Anti-ROR1-VH(2)
Anti-ROR1-VL(14) and Anti-ROR1-VH(3)
Anti-ROR1-VL(14) and Anti-ROR1-VH(4)
Anti-ROR1-VL(14) and Anti-ROR1-VH(5)
Anti-ROR1-VL(14) and Anti-ROR1-VH(6)
Anti-ROR1-VL(14) and Anti-ROR1-VH(7)
Anti-ROR1-VL(14) and Anti-ROR1-VH(8)
Anti-ROR1-VL(14) and Anti-ROR1-VH(9)

In various embodiments, the ROR1-binding molecules of the present invention comprise one of the following combinations:

anti-ROR1-VL(2) and anti-ROR1-VH(1)
anti-ROR1-VL(2) and anti-ROR1-VH(2)
anti-ROR1-VL(2) and anti-ROR1-VH(3)
anti-ROR1-VL(2) and anti-ROR1-VH(4)
anti-ROR1-VL(2) and anti-ROR1-VH(7)
anti-ROR1-VL(2) and anti-ROR1-VH(8)
anti-ROR1-VL(2) and anti-ROR1-VH(9)
anti-ROR1-VL(11) and anti-ROR1-VH(1)
anti-ROR1-VL(11) and anti-ROR1-VH(2)
anti-ROR1-VL(11) and anti-ROR1-VH(3)
anti-ROR1-VL(11) and anti-ROR1-VH(4)
anti-ROR1-VL(11) and anti-ROR1-VH(7)
anti-ROR1-VL(11) and anti-ROR1-VH(8)
anti-ROR1-VL(11) and anti-ROR1-VH(9)
anti-ROR1-VL(12) and anti-ROR1-VH(1)
anti-ROR1-VL(12) and anti-ROR1-VH(2)
anti-ROR1-VL(12) and anti-ROR1-VH(3)
anti-ROR1-VL(12) and anti-ROR1-VH(4)
anti-ROR1-VL(12) and anti-ROR1-VH(7)
anti-ROR1-VL(12) and anti-ROR1-VH(8)
anti-ROR1-VL(12) and anti-ROR1-VH(9)
anti-ROR1-VL(13) and anti-ROR1-VH(1)
anti-ROR1-VL(13) and anti-ROR1-VH(2)
anti-ROR1-VL(13) and anti-ROR1-VH(3)
anti-ROR1-VL(13) and anti-ROR1-VH(4)
anti-ROR1-VL(13) and anti-ROR1-VH(7)
anti-ROR1-VL(13) and anti-ROR1-VH(8)
anti-ROR1-VL(13) and anti-ROR1-VH(9)
anti-ROR1-VL(14) and anti-ROR1-VH(1)
anti-ROR1-VL(14) and anti-ROR1-VH(2)
anti-ROR1-VL(14) and anti-ROR1-VH(3)
anti-ROR1-VL(14) and anti-ROR1-VH(4)
anti-ROR1-VL(14) and anti-ROR1-VH(7)
anti-ROR1-VL(14) and anti-ROR1-VH(8)
anti-ROR1-VL(14) and anti-ROR1-VH(9)

Particularly preferred combinations are:

anti-ROR1-VL(2) and anti-ROR1-VH(3)
anti-ROR1-VL(2) and anti-ROR1-VH(7)
anti-ROR1-VL(2) and anti-ROR1-VH(8)
anti-ROR1-VL(2) and anti-ROR1-VH(9)
anti-ROR1-VL(14) and anti-ROR1-VH(3)
anti-ROR1-VL(14) and anti-ROR1-VH(7)
anti-ROR1-VL(14) and anti-ROR1-VH(8)
anti-ROR1-VL(14) and anti-ROR1-VH(9)

The present invention specifically encompasses ROR1-binding molecules comprising (i) an optimized anti-ROR1-VL and/or VH Domain as provided above, and (ii) an Fc Region. In particular embodiments, the ROR1-binding molecules of the present invention are monoclonal antibodies comprising (i) an optimized anti-ROR1-VL and/or VH Domain as provided above, and (ii) an Fc Region. In other embodiments, the ROR1-binding molecules of the present invention are selected from the group consisting of: monoclonal antibodies, multispecific antibodies, synthetic antibodies, chimeric antibodies, single-chain Fvs (scFv), single-chain antibodies, Fab fragments, F(ab′) fragments, disulfide-linked bispecific Fvs (sdFv), BiTEs, diabodies, and trivalent binding molecules.

### V. Chimeric Antigen Receptors

The ROR1-binding molecules of the present invention may be monospecific single-chain molecules such as single-chain variable fragments (“anti-ROR1-scFvs”) or Chimeric Antigen Receptors (“anti-ROR1-CARs”). As discussed above, scFvs are made by linking Light and Heavy Chain Variable Domains together via a short linking peptide. First-generation CARs typically had the intracellular domain from the CD3 ζ-chain, which is the primary transmitter of signals from endogenous TCRs. Second-generation CARs possessed additional intracellular signaling domains from various costimulatory protein receptors (e.g., CD28, 41BB, ICOS, etc.) to the cytoplasmic tail of the CAR in order to provide additional signals to the T-cell. Third-generation CARs combine multiple signaling domains, such as CD3z-CD28-41BB or CD3z-CD28-OX_{40,} in order to further augment potency (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor-Modified T Cells,” Blood 123(15): 2343-2354; Mardiros, A. et al. (2013) “T Cells Expressing CD123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD 123 Antigens Efficiently Target Primary Acute Myeloid Leukemia Cells in vivo,” Leukemia doi:10.1038/leu.2014.62).

The anti-ROR1-CARs of the present invention comprise an anti-ROR1-scFv fused to an intracellular domain of a receptor. The Variable Light Chain and Variable Heavy Chain Domains of the anti-ROR1-scFv are selected from any of the optimized anti-ROR1-VL and anti-ROR1-VH Domains disclosed herein. Preferably, the VL Domain is selected from the group consisting of: anti-ROR1-VL(2) (SEQ ID NO:11), anti-ROR1-VL(11) (SEQ ID NO:20), anti-ROR1-VL(12) (SEQ ID NO:21), anti-ROR1-VL(13) (SEQ ID NO:22), and anti-ROR1-VL(14) (SEQ ID NO:23). Preferably, the VH Domain is selected from the group consisting of: anti-ROR1-VH(3) (SEQ ID NO:26), anti-ROR1-VH(7) (SEQ ID NO:30), anti-ROR1-VH(8) (SEQ ID NO:31), and anti-ROR1-VH(9) (SEQ ID NO:32). Thus, the following combinations of optimized anti-ROR1-VL and anti-ROR1-VH Domains are preferred for such anti-ROR1-scFvs of such anti-ROR1-CARs:

anti-ROR1-VL(2) and anti-ROR1-VH(3)
anti-ROR1-VL(2) and anti-ROR1-VH(7)
anti-ROR1-VL(2) and anti-ROR1-VH(8)
anti-ROR1-VL(2) and anti-ROR1-VH(9)
anti-ROR1-VL(11) and anti-ROR1-VH(3)
anti-ROR1-VL(11) and anti-ROR1-VH(7)
anti-ROR1-VL(11) and anti-ROR1-VH(8)
anti-ROR1-VL(11) and anti-ROR1-VH(9)
anti-ROR1-VL(12) and anti-ROR1-VH(3)
anti-ROR1-VL(12) and anti-ROR1-VH(7)
anti-ROR1-VL(12) and anti-ROR1-VH(8)
anti-ROR1-VL(12) and anti-ROR1-VH(9)
anti-ROR1-VL(13) and anti-ROR1-VH(3)
anti-ROR1-VL(13) and anti-ROR1-VH(7)
anti-ROR1-VL(13) and anti-ROR1-VH(8)
anti-ROR1-VL(13) and anti-ROR1-VH(9)
anti-ROR1-VL(14) and anti-ROR1-VH(3)
anti-ROR1-VL(14) and anti-ROR1-VH(7)
anti-ROR1-VL(14) and anti-ROR1-VH(8)
anti-ROR1-VL(14) and anti-ROR1-VH(9)

The intracellular domain of the anti-ROR1-CARs of the present invention is preferably selected from the intracellular domain of any of: 41BB-CD3ζ, b2c-CD3ζ, CD28, CD28-4-1BB-CD3ζ, CD28-CD3ζ, CD28-FcεRIγ, CD28mut-CD3ζ, CD28-OX40-CD3ζ, CD28-OX40-CD3ζ, CD3ζ, CD4-CD3ζ, CD4-FcεRIγ, CD8-CD3ζ, FcεRIγ, FcεRIγCAIX, Heregulin-CD3ζ, IL-13-CD3ζ, or Ly49H-CD3ζ t (Tettamanti, S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401; Gill, S. et al. (2014) “Efficacy Against Human Acute Myeloid Leukemia And Myeloablation Of Normal Hematopoiesis In A Mouse Model Using Chimeric Antigen Receptor-Modified T Cells,” Blood 123(15): 2343-2354; Mardiros, A. et al. (2013) “T Cells Expressing CD 123-Specific Chimeric Antigen Receptors Exhibit Specific Cytolytic Effector Functions And Antitumor Effects Against Human Acute Myeloid Leukemia,” Blood 122:3138-3148; Pizzitola, I. et al. (2014) “Chimeric Antigen Receptors Against CD33/CD 123 Antigens Efficiently Target Primary Acute Myeloid Leukemia Cells in vivo,” Leukemia doi : 10.1038/1eu.2014. 62).

### VI. Multispecific ROR1-Binding Molecules

The present invention is also directed to ROR1-binding molecules comprising an epitope-binding site (preferably comprising an optimized anti-ROR1-VL Domain of the invention and/or an optimized anti-ROR1-VH Domain of the invention) and further comprising a second epitope-binding site that immunospecifically binds to a second epitope, where such second epitope is (i) a different epitope of ROR1, or (ii) an epitope of a molecule that is not ROR1. Such trispecific or multispecific ROR1-binding molecules preferably comprise a combination of epitope-binding sites that recognize a set of antigens unique to target cells or tissue type. In particular, the present invention relates to trispecific or multispecific ROR1-binding molecules that are capable of binding to an epitope of ROR1 and an epitope of a molecule present on the surface of an effector cell, especially a T lymphocyte, a natural killer (NK) cell or other mononuclear cell. For example, such ROR1-binding molecules of the present invention may be constructed to comprise an epitope-binding site that immunospecifically binds CD2, CD3, CD8, CD16, T-Cell Receptor (TCR), or NKG2D.

One embodiment of the present invention relates to bispecific ROR1-binding molecules that are capable of binding to a “first epitope” and a “second epitope,” such epitopes not being identical to one another. Such bispecific molecules comprise “VL1”/“VH1” domains that are capable of binding to the first epitope, and “VL2”/“VH2” domains that are capable of binding to the second epitope. The notation “VL1” and “VH1” denote respectively, the Variable Light Chain Domain and Variable Heavy Chain Domain that bind the “first” epitope of such bispecific molecules. Similarly, the notation “VL2” and “VH2” denote respectively, the Light Chain Variable Domain and Heavy Chain Variable Domain that bind the “second” epitope of such bispecific molecules. It is irrelevant whether a particular epitope is designated as the first vs. the second epitope; such notation having relevance only with respect to the presence and orientation of domains of the polypeptide chains of the binding molecules of the present invention. In one embodiment, one of such epitopes is an epitope of human ROR1 and the other is a different epitope of ROR1, or is an epitope of a molecule that is not ROR1. In particular embodiments, one of such epitopes is an epitope of human ROR1 and the other is an epitope of a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, a bispecific molecule comprises more than two epitope-binding sites. Such bispecific molecules will bind at least one epitope of ROR1 and at least one epitope of a molecule that is not ROR1, and may further bind additional epitopes of ROR1 and/or additional epitopes of a molecule that is not ROR1.

The present invention particularly relates to bispecific, trispecific and multispecific ROR1-binding molecules (e.g., bispecific antibodies, bispecific diabodies, trivalent binding molecules, etc.) that possess epitope-binding fragments of antibodies (e.g., VL and VH Domains) that enable them to be able to coordinately bind to at least one epitope of ROR1 and at least one epitope of a second molecule that is not ROR1. Selection of the VL and VH Domains of the polypeptide domains of such molecules is coordinated so that the polypeptides chains that make up such multispecific ROR1-binding molecules assemble to form at least one functional epitope-binding site that is specific for at least one epitope of ROR1 and at least one functional epitope-binding site that is specific for at least one epitope of a molecule that is not ROR1. Preferably, the bispecific ROR1-binding molecules comprise an optimized anti-ROR1-VL and/or VH Domain as provided herein.

A. Bispecific Antibodies

The instant invention encompasses bispecific antibodies capable of simultaneously binding to an epitope of ROR1 and an epitope of a molecule that is not ROR1. In some embodiments, the bispecific antibody capable of simultaneously binding to ROR1 and a second molecule that is not ROR1 is produced using any of the methods described in PCT Publication Nos. WO 1998/002463, WO 2005/070966, WO 2006/107786 WO 2007/024715, WO 2007/075270, WO 2006/107617, WO 2007/046893, WO 2007/146968, WO 2008/003103, WO 2008/003116, WO 2008/027236, WO 2008/024188, WO 2009/132876, WO 2009/018386, WO 2010/028797, WO2010028796, WO 2010/028795, WO 2010/108127, WO 2010/136172, WO 2011/086091, WO 2011/133886, WO 2012/009544, WO 2013/003652, WO 2013/070565, WO 2012/162583, WO 2012/156430, WO 2013/174873, and WO 2014/022540, each of which is hereby incorporated herein by reference in its entirety.

B. Bispecific Diabodies Lacking Fc Regions

One embodiment of the present invention relates to bispecific diabodies that are capable of binding to a first epitope and a second epitope, wherein the first epitope is an epitope of human ROR1 and the second is an epitope of a molecule that is not ROR1, preferably a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. Such diabodies comprise, and most preferably are composed of, a first polypeptide chain and a second polypeptide chain, whose sequences permit the polypeptide chains to covalently bind to each other to form a covalently associated diabody that is capable of simultaneously binding to an epitope of ROR1 and the second epitope.

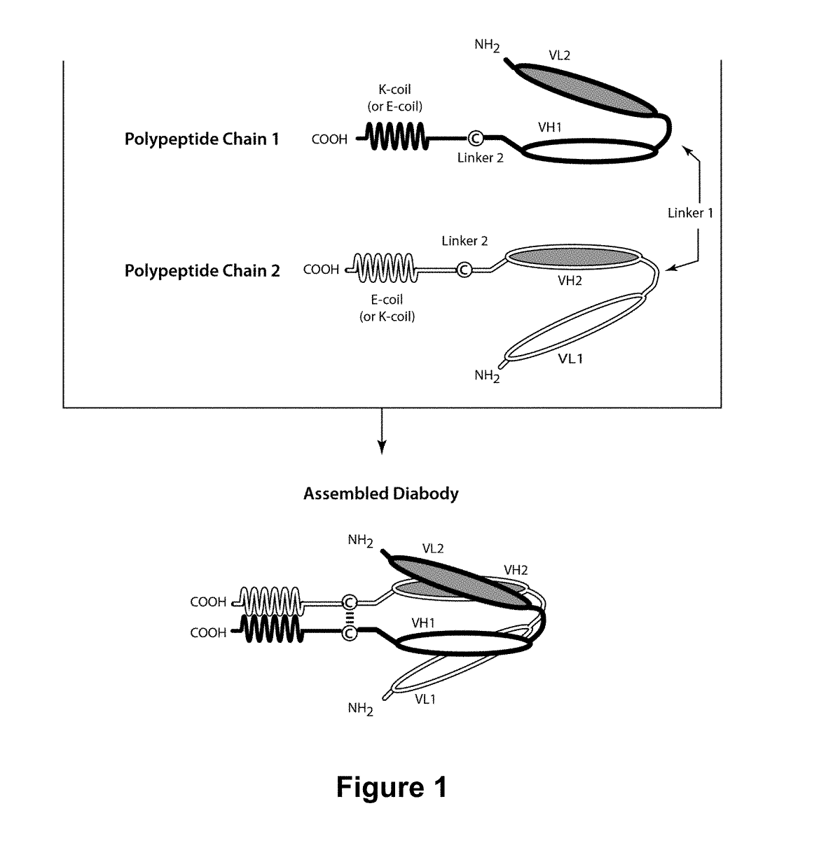

The first polypeptide chain of such an embodiment of bispecific diabodies comprises, in the N-terminal to C-terminal direction: an N-terminus, the VL Domain of a monoclonal antibody capable of binding to either the first or second epitope (i.e., either VL_{anti-ROR1-VL} or VL_{Epitope 2}), a first intervening spacer peptide (Linker 1), a VH Domain of a monoclonal antibody capable of binding to either the second epitope (if such first polypeptide chain contains VL_{anti-ROR1-VL}) or ROR1 (if such first polypeptide chain contains VL_{Epitope 2}), a second intervening spacer peptide (Linker 2) optionally containing a cysteine residue, a Heterodimer-Promoting Domain and a C-terminus (FIG. 1).

The second polypeptide chain of this embodiment of bispecific diabodies comprises, in the N-terminal to C-terminal direction: an N-terminus, a VL Domain of a monoclonal antibody capable of binding to either the first or second epitope (i.e., either VL_{anti-ROR1-VL} or VL_{Epitope 2} , and being the VL Domain not selected for inclusion in the first polypeptide chain of the diabody), an intervening spacer peptide (Linker 1), a VH Domain of a monoclonal antibody capable of binding to either the second epitope (if such second polypeptide chain contains VL_{anti-ROR1-VL}) or to ROR1 (if such second polypeptide chain contains VL_{Epitope 2}), a second intervening spacer peptide (Linker 2) optionally containing a cysteine residue, a Heterodimer-Promoting Domain, and a C-terminus (FIG. 1).

The VL Domain of the first polypeptide chain interacts with the VH Domain of the second polypeptide chain to form a first functional epitope-binding site that is specific for a first antigen (i.e., either ROR1 or a molecule that contains the second epitope). Likewise, the VL Domain of the second polypeptide chain interacts with the VH Domain of the first polypeptide chain in order to form a second functional epitope-binding site that is specific for a second antigen (i.e., either the molecule that comprises the second epitope or ROR1). Thus, the selection of the VL and VH Domains of the first and second polypeptide chains is coordinated, such that the two polypeptide chains of the diabody collectively comprise VL and VH Domains capable of binding to both an epitope of ROR1 and to the second epitope (i.e., they collectively comprise VL_{anti-ROR1-VL}/VH_{anti-ROR1-VH} and VL_{Epitope 2}/VH_{Epitope 2}).

Most preferably, the length of the intervening spacer peptide (i.e., “Linker 1,” which separates such VL and VH Domains) is selected to substantially or completely prevent the VL and VH Domains of the polypeptide chain from binding to one another (for example consisting of from 0, 1, 2, 3, 4, 5, 6, 7, 8 or 9 intervening linker amino acid residues). Thus the VL and VH Domains of the first polypeptide chain are substantially or completely incapable of binding to one another. Likewise, the VL and VH Domains of the second polypeptide chain are substantially or completely incapable of binding to one another. A preferred intervening spacer peptide (Linker 1) has the sequence (SEQ ID NO:33): GGGSGGGG.

The length and composition of the second intervening spacer peptide (“Linker 2”) is selected based on the choice of one or more polypeptide domains that promote such dimerization (i.e., a “Heterodimer-Promoting Domain”). Typically, the second intervening spacer peptide (Linker 2) will comprise 3-20 amino acid residues. In particular, where the employed Heterodimer-Promoting Domain(s) do/does not comprise a cysteine residue a cysteine-containing second intervening spacer peptide (Linker 2) is utilized. A cysteine-containing second intervening spacer peptide (Linker 2) will contain 1, 2, 3 or more cysteines. A preferred cysteine-containing spacer peptide (Linker 2) has the sequence GGCGGG (SEQ ID NO:34). Alternatively, Linker 2 does not comprise a cysteine (e.g., GGG, GGGS (SEQ ID NO:35), LGGGSG (SEQ ID NO:36), GGGSGGGSGGG (SEQ ID NO:37), ASTKG (SEQ ID NO:38), LEPKSS (SEQ ID NO:39), APSSS (SEQ ID NO:40), etc.) and a Cysteine-Containing Heterodimer-Promoting Domain, as described below is used. Optionally, both a cysteine-containing Linker 2 and a cysteine-containing Heterodimer-Promoting Domain are used.

The Heterodimer-Promoting Domains may be GVEPKSC (SEQ ID NO:41) or VEPKSC (SEQ ID NO:42) or AEPKSC (SEQ ID NO:43) on one polypeptide chain and GFNRGEC (SEQ ID NO:44) or FNRGEC (SEQ ID NO:45) on the other polypeptide chain (US2007/0004909).

In a preferred embodiment, the Heterodimer-Promoting Domains will comprise tandemly repeated coil domains of opposing charge for example, “E-coil” helical domains (SEQ ID NO:46: EVAALEK-EVAALEK-EVAALEK-EVAALEK), whose glutamate residues will form a negative charge at pH 7, and “K-coil” domains (SEQ ID NO:47: KVAALKE-KVAALKE-KVAALKE-KVAALKE), whose lysine residues will form a positive charge at pH 7. The presence of such charged domains promotes association between the first and second polypeptides, and thus fosters heterodimer formation. Heterodimer-Promoting Domains that comprise modifications of the above-described E-coil and K-coil sequences so as to include one or more cysteine residues may be utilized. The presence of such cysteine residues permits the coil present on one polypeptide chain to become covalently bonded to a complementary coil present on another polypeptide chain, thereby covalently bonding the polypeptide chains to one another and increasing the stability of the diabody. Examples of such particularly preferred are Heterodimer-Promoting Domains include a Modified E-Coil having the amino acid sequence EVAACEK-EVAALEK-EVAALEK-EVAALEK (SEQ ID NO:48), and a modified K-coil having the amino acid sequence KVAACKE-KVAALKE-KVAALKE-KVAALKE (SEQ ID NO:49).

As disclosed in WO 2012/018687, in order to improve the in vivo pharmacokinetic properties of diabodies, a diabody may be modified to contain a polypeptide portion of a serum-binding protein at one or more of the termini of the diabody. Most preferably, such polypeptide portion of a serum-binding protein will be installed at the C-terminus of a polypeptide chain of the diabody. Albumin is the most abundant protein in plasma and has a half-life of 19 days in humans. Albumin possesses several small molecule binding sites that permit it to non-covalently bind to other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120. Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of protein G of Streptococcus strain G148 (SEQ ID NO:50): LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:50 have the ability to attenuate or eliminate MHC class II binding. Based on combinational mutation results, the following combinations of substitutions are considered to be preferred substitutions for forming such a deimmunized ABD: 66D/70S+71A; 66S/70S+71A; 66S/70S+79A; 64A/65A/71A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 51)

LAEAKVLANR ELDKYGVSDY YKNLID₆₆NAKS₇₀ A₇₁EGVKALIDE
ILAALP,
or the amino acid sequence:

(SEQ ID NO: 52)

LAEAKVLANR ELDKYGVSDY YKNA₆₄A₆₅NNAKT VEGVKALIA₇₉E
ILAALP,
or the amino acid sequence:

(SEQ ID NO: 53)

LAEAKVLANR ELDKYGVSDY YKNLIS₆₆NAKS₇₀ VEGVKALIA₇₉E
ILAALP,

are particularly preferred as such deimmunized ABD exhibit substantially wild-type binding while providing attenuated MHC class II binding. Thus, the first polypeptide chain of such a diabody having an ABD contains a third linker (Linker 3) preferably positioned C-terminally to the E-coil (or K-coil) Domain of such polypeptide chain so as to intervene between the E-coil (or K-coil) Domain and the ABD (which is preferably a deimmunized ABD). A preferred sequence for such Linker 3 is SEQ ID NO:35: GGGS.

C. Multispecific Diabodies Containing Fc Regions

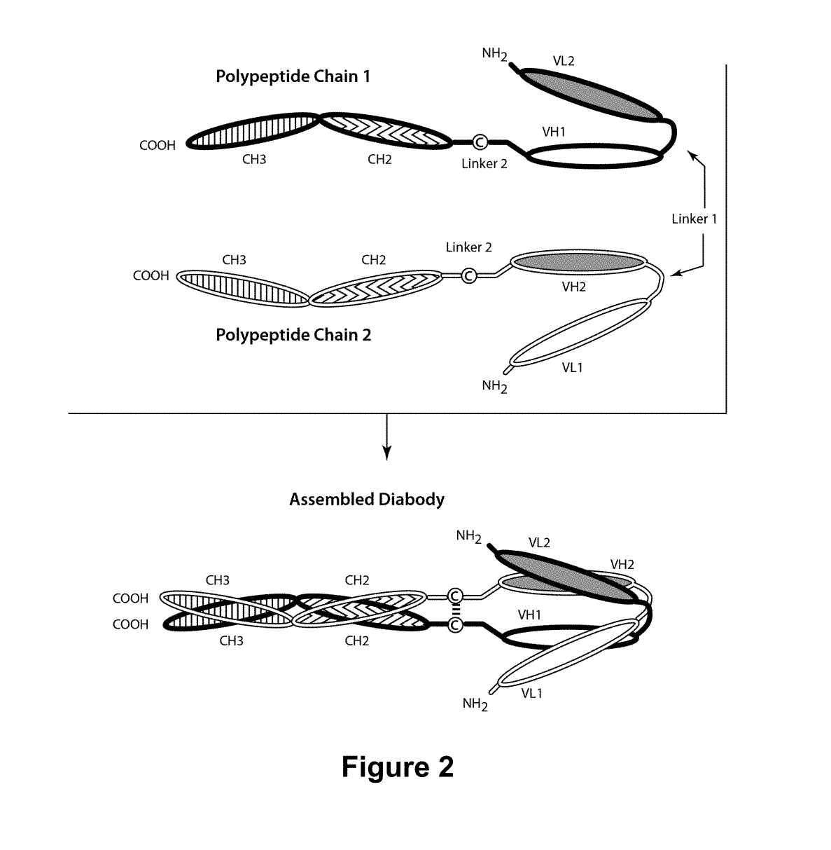

One embodiment of the present invention relates to multispecific diabodies capable of simultaneously binding to an epitope of ROR1 and a second epitope (i.e., a different epitope of ROR1 or an epitope of a molecule that is not ROR1) that comprise an Fc Region. The addition of an IgG CH2-CH3 Domain to one or both of the diabody polypeptide chains, such that the complexing of the diabody chains results in the formation of an Fc Region, increases the biological half-life and/or alters the valency of the diabody. Such diabodies comprise, two or more polypeptide chains whose sequences permit the polypeptide chains to covalently bind to each other to form a covalently associated diabody that is capable of simultaneously binding to an epitope of ROR1 and the second epitope. Incorporating an IgG CH2-CH3 Domains onto both of the diabody polypeptides will permit a two-chain bispecific Fc-Region-containing diabody to form (FIG. 2).

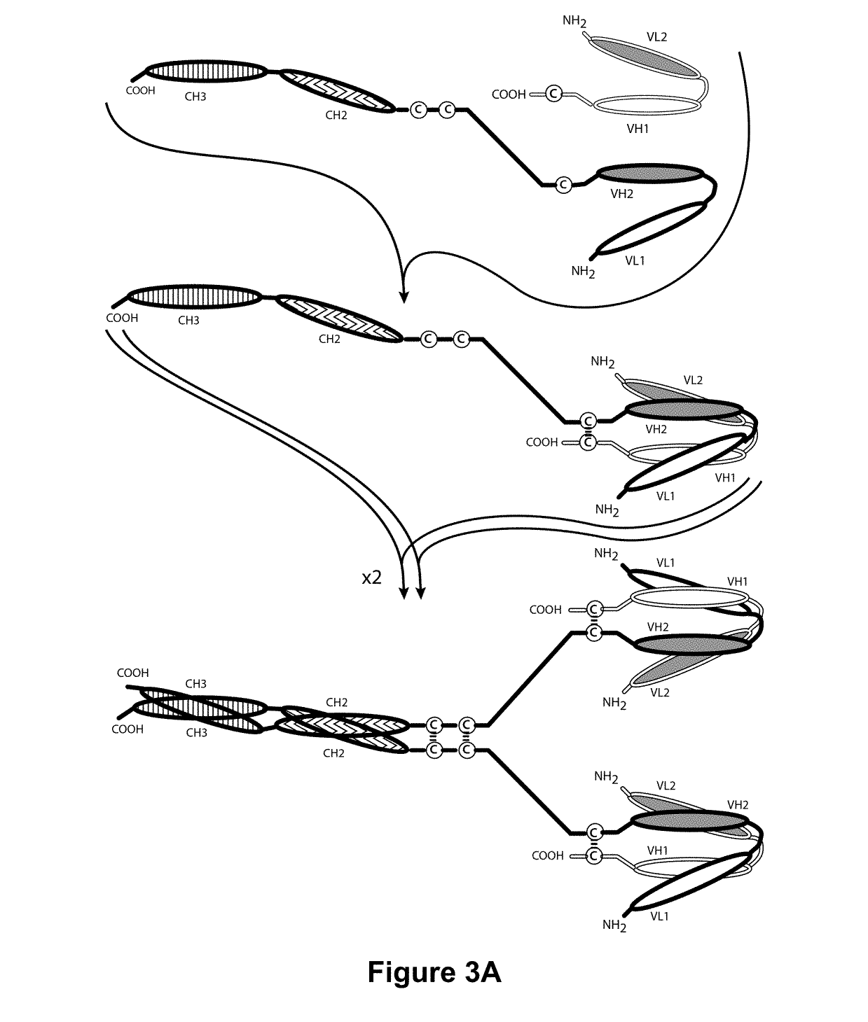

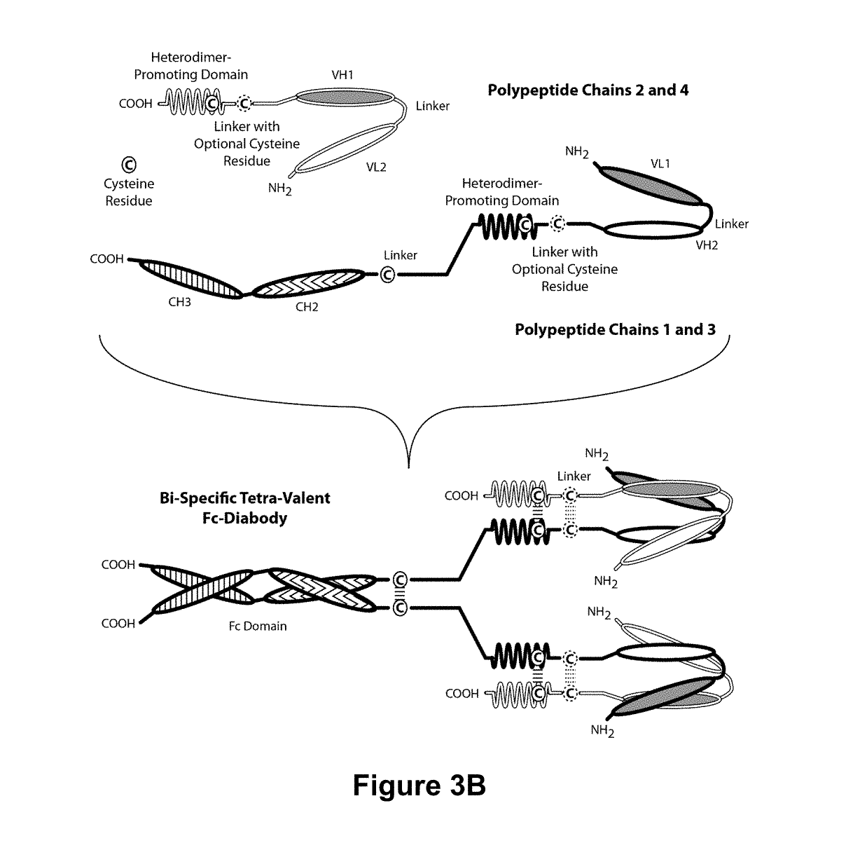

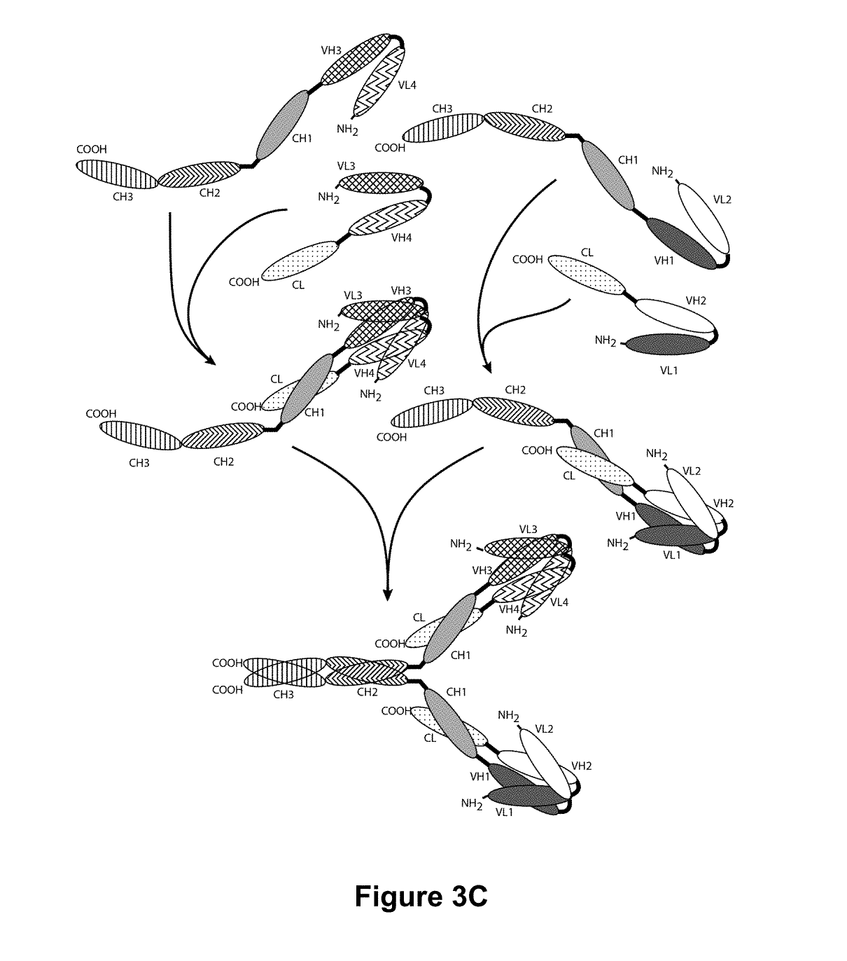

Alternatively, incorporating an IgG CH2-CH3 Domains onto only one of the diabody polypeptides will permit a more complex four-chain bispecific Fc Region-containing diabody to form (FIGS. 3A-3C). FIG. 3C shows a representative four-chain diabody possessing the Constant Light (CL) Domain and the Constant Heavy CH1 Domain, however fragments of such domains as well as other polypeptides may alternatively be employed (see, e.g., FIGS. 3A and 3B, United States Patent Publication Nos. 2013-0295121; 2010-0174053 and 2009-0060910; European Patent Publication No. EP 2714079; EP 2601216; EP 2376109; EP 2158221 and PCT Publication Nos. WO 2012/162068; WO 2012/018687; WO 2010/080538). Thus, for example, in lieu of the CH1 Domain, one may employ a peptide having the amino acid sequence GVEPKSC (SEQ ID NO:41), VEPKSC (SEQ ID NO:42), or AEPKSC (SEQ ID NO:43), derived from the Hinge Region of a human IgG, and in lieu of the CL Domain, one may employ the C-terminal 6 amino acids of the human kappa light chain, GFNRGEC (SEQ ID NO:44) or FNRGEC (SEQ ID NO:45). A representative peptide containing four-chain diabody is shown in FIG. 3A. Alternatively, or in addition, one may employ a peptide comprising tandem coil domains of opposing charge such as the “E-coil” helical domains (SEQ ID NO:46: EVAALEK-EVAALEK-EVAALEK-EVAALEK or SEQ ID NO:48: EVAACEK-EVAALEK-EVAALEK-EVAALEK); and the “K-coil” domains (SEQ ID NO:47: KVAALKE-KVAALKE-KVAALKE-KVAALKE or SEQ ID NO:49: KVAACKE-KVAALKE-KVAALKE-KVAALKE). A representative coil domain containing four-chain diabody is shown in FIG. 3B.

The Fc Region-containing molecules of the present invention may include additional intervening spacer peptides (Linkers), generally such Linkers will be incorporated between a Heterodimer-Promoting Domain (e.g., an E-coil or K-coil) and a CH2-CH3 Domain and/or between a CH2-CH3 Domain and a Variable Domain (i.e., VH or VL). Typically, the additional Linkers will comprise 3-20 amino acid residues and may optionally contain all or a portion of an IgG Hinge Region (preferably a cysteine-containing portion of an IgG Hinge Region). Linkers that may be employed in the bispecific Fc Region-containing diabody molecules of the present invention include: GGGS (SEQ ID NO:35), LGGGSG (SEQ ID NO:36), GGGSGGGSGGG (SEQ ID NO:37), ASTKG (SEQ ID NO:38), LEPKSS (SEQ ID NO:39), APSSS (SEQ ID NO:40), APSSSPME (SEQ ID NO:54), VEPKSADKTHTCPPCP (SEQ ID NO:55), LEPKSADKTHTCPPCP (SEQ ID NO:56), DKTHTCPPCP (SEQ ID NO:57), GGC, and GGG. LEPKSS (SEQ ID NO:39) may be used in lieu of GGG or GGC for ease of cloning. Additionally, the amino acids GGG, or LEPKSS (SEQ ID NO:39) may be immediately followed by DKTHTCPPCP (SEQ ID NO:57) to form the alternate linkers: GGGDKTHTCPPCP (SEQ ID NO:58); and LEPKSSDKTHTCPPCP (SEQ ID NO:59). Bispecific Fc Region-containing molecules of the present invention may incorporate an IgG Hinge Region in addition to or in place of a linker. Exemplary Hinge Regions include: EPKSCDKTHTCPPCP (SEQ ID NO:60) from IgG1, ERKCCVECPPCP (SEQ ID NO:61) from IgG2, ELKTPLGDTTHTCPRCPEPKSCDTPPPCPRCPEPKSCDTPPPCPRCPEPKSCDTPPPCPRCP (SEQ ID NO:116) from IgG3, ESKYGPPCPSCP (SEQ ID NO:62) from IgG4, and ESKYGPPCPPCP (SEQ ID NO:63) an IgG4 hinge variant comprising a stabilizing S228P substitution (as numbered by the EU index as set forth in Kabat) to reduce strand exchange.

As provided in FIG. 3A-3C, Fc Region-containing diabodies of the invention may comprise four chains. The first and third polypeptide chains of such a diabody contain three domains: (i) a VL1-containing Domain, (ii) a VH2-containing Domain, (iii) a Heterodimer-Promoting Domain, and (iv) a Domain containing a CH2-CH3 sequence. The second and fourth polypeptide chains contain: (i) a VL2-containing Domain, (ii) a VH1-containing Domain, and (iii) a Heterodimer-Promoting Domain, where the Heterodimer-Promoting Domains promote the dimerization of the first/third polypeptide chains with the second/fourth polypeptide chains. The VL and/or VH Domains of the third and fourth polypeptide chains, and VL and/or VH Domains of the first and second polypeptide chains may be the same or different so as to permit tetravalent binding that is either monospecific, bispecific or tetraspecific. The notation “VL3” and “VH3” denote respectively, the Light Chain Variable Domain and Variable Heavy Chain Domain that bind a “third” epitope of such diabody. Similarly, the notation “VL4” and “VH4” denote respectively, the Light Chain Variable Domain and Variable Heavy Chain Domain that bind a “fourth” epitope of such diabody. The general structure of the polypeptide chains of a representative four-chain bispecific Fc Region-containing diabodies of invention is provided in Table 1:

TABLE 1

| | | |
|---|---|---|
| Bispecific | 2^{nd} Chain | NH₂-L2-VH1-HPD-COOH |
| | 1^{st} Chain | NH₂-VL1-VH2-HPD-CH2—CH3—COOH |
| | 1^{st} Chain | NH₂-VL1-VH2-HPD-CH2—CH3—COOH |
| | 2^{nd} Chain | NH₂-VL2-VH1-HPD-COOH |
| Tetraspecific | 2^{nd} Chain | NH₂-VL2-VH1-HPD-COOH |
| | 1^{st} Chain | NH₂-VL1-VH2-HPD-CH2—CH3—COOH |
| | 3^{rd} Chain | NH₂-VL3-VH4-HPD-CH2—CH3—COOH |
| | 4^{th} Chain | NH₂-VL4-VH3-HPD-COOH |

HPD = Heterodimer-Promoting Domain

In a specific embodiment, diabodies of the present invention are bispecific, tetravalent (i.e., possess four epitope-binding sites), Fc-containing diabodies that are composed of four total polypeptide chains (FIGS. 3A-3C). The bispecific, tetravalent, Fc-containing diabodies of the invention comprise two epitope-binding sites immunospecific for ROR1 (which may be capable of binding to the same epitope of ROR1 or to different epitopes of ROR1), and two epitope-binding sites immunospecific for a second molecule (which may be capable of binding to the same epitope of the second molecule or to different epitopes of the second molecule). Preferably, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell.

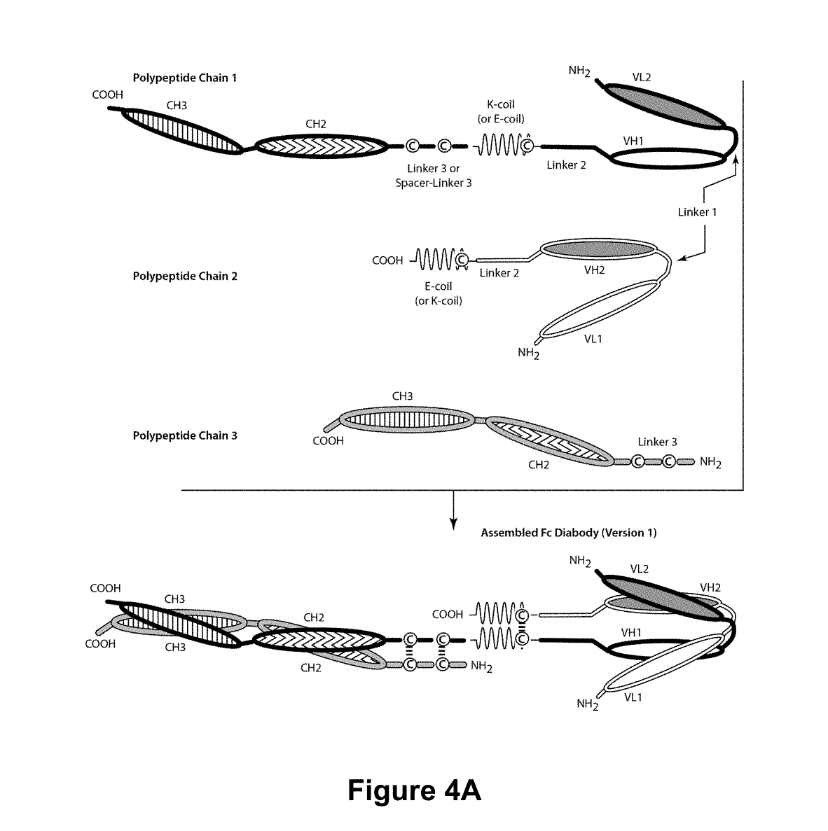

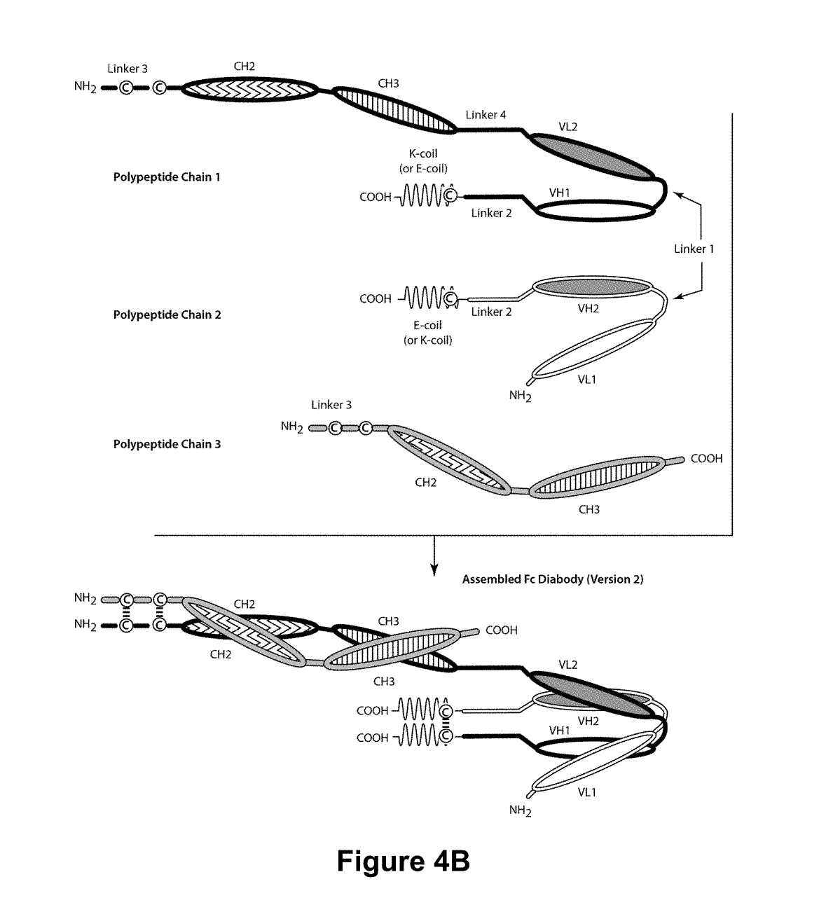

In a further embodiment, the Fc Region-containing diabodies of the present invention may comprise three polypeptide chains. The first polypeptide of such a diabody contains three domains: (i) a VL1-containing Domain, (ii) a VH2-containing Domain and (iii) a Domain containing a CH2-CH3 sequence. The second polypeptide of such a diabody contains: (i) a VL2-containing Domain, (ii) a VH1-containing Domain and (iii) a Domain that promotes heterodimerization and covalent bonding with the diabody's first polypeptide chain. The third polypeptide of such a diabody comprises a CH2-CH3 sequence. Thus, the first and second polypeptide chains of such a diabody associate together to form a VL1/VH1 epitope-binding site that is capable of binding to a first antigen (i.e., either ROR1 or a molecule that comprises a second epitope), as well as a VL2/VH2 epitope-binding site that is capable of binding to a second antigen (i.e., either the molecule that contains the second epitope or ROR1). The first and second polypeptides are bonded to one another through a disulfide bond involving cysteine residues in their respective Third Domains. Notably, the first and third polypeptide chains complex with one another to form an Fc Region that is stabilized via a disulfide bond. Such bispecific diabodies have enhanced potency. FIGS. 4A and 4B illustrate the structures of such diabodies. Such Fc-Region-containing diabodies may have either of two orientations (Table 2):

TABLE 2

| | | |
|---|---|---|
| First | 3^{rd} Chain | NH₂—CH2—CH3—COOH |
| Orientation | 1^{st} Chain | NH₂-VL1-VH2-HPD-CH2—CH3—COOH |
| | 2^{nd} Chain | NH₂-VL2-VH1-HPD-COOH |
| Second | 3^{rd} Chain | NH₂—CH2—CH3—COOH |
| Orientation | 1^{st} Chain | NH₂—CH2—CH3-VL1-VH2-HPD-COOH |
| | 2^{nd} Chain | NH₂-VL2-VH1-HPD-COOH |

HPD = Heterodimer-Promoting Domain

In a specific embodiment, diabodies of the present invention are bispecific, bivalent (i.e., possess two epitope-binding sites), Fc-containing diabodies that are composed of three total polypeptide chains (FIGS. 4A-4B). The bispecific, bivalent Fc-containing diabodies of the invention comprise one epitope-binding site immunospecific for ROR1, and one epitope-binding site immunospecific for a second molecule. Preferably, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell.

In a further embodiment, the Fc Region-containing diabodies may comprise a total of five polypeptide chains. In a particular embodiment, two of said five polypeptide chains have the same amino acid sequence. The first polypeptide chain of such a diabody contains: (i) a VH1-containing domain, (ii) a CH1-containing domain, and (iii) a Domain containing a CH2-CH3 sequence. The first polypeptide chain may be the heavy chain of an antibody that contains a VH1 and a heavy chain constant region. The second and fifth polypeptide chains of such a diabody contain: (i) a VL1-containing domain, and (ii) a CL-containing domain. The second and/or fifth polypeptide chains of such a diabody may be light chains of an antibody that contains a VL1 complementary to the VH1 of the first/third polypeptide chain. The first, second and/or fifth polypeptide chains may be isolated from a naturally occurring antibody. Alternatively, they may be constructed recombinantly. The third polypeptide chain of such a diabody contains: (i) a VH1-containing domain, (ii) a CH1-containing domain, (iii) a Domain containing a CH2-CH3 sequence, (iv) a VL2-containing Domain, (v) a VH3-containing Domain and (vi) a Heterodimer-Promoting Domain, where the Heterodimer-Promoting Domains promote the dimerization of the third chain with the fourth chain. The fourth polypeptide of such diabodies contains: (i) a VL3-containing Domain, (ii) a VH2-containing Domain and (iii) a Domain that promotes heterodimerization and covalent bonding with the diabody's third polypeptide chain.

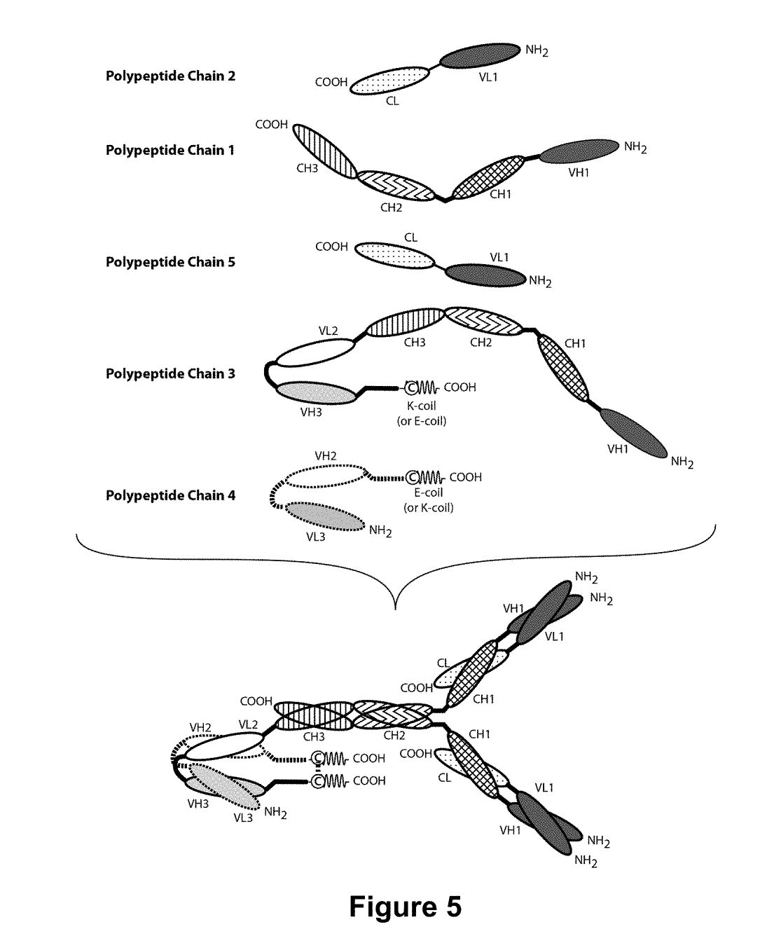

Thus, the first and second, and the third and fifth, polypeptide chains of such diabodies associate together to form two VL1/VH1 epitope-binding sites capable of binding a first epitope. The third and fourth polypeptide chains of such diabodies associate together to form a VL2/VH2 epitope-binding site that is capable of binding to a second epitope, as well as a VL3/VH3 binding site that is capable of binding to a third epitope. The first and third polypeptides are bonded to one another through a disulfide bond involving cysteine residues in their respective constant regions. Notably, the first and third polypeptide chains complex with one another to form an Fc Region. Such multispecific diabodies have enhanced potency. FIG. 5 illustrates the structure of such diabodies. It will be understood that the VL1/VH1, VL2/VH2, and VL3/VH3 Domains may be the same or different so as to permit binding that is monospecific, bispecific or trispecific. As provided herein, these domains are preferably selected so as to bind an epitope of ROR1, an epitope of second molecule and optionally an epitope of a third molecule.

The VL and VH Domains of the polypeptide chains are selected so as to form VL/VH binding sites specific for a desired epitope. The VL/VH binding sites formed by the association of the polypeptide chains may be the same or different so as to permit tetravalent binding that is monospecific, bispecific, trispecific or tetraspecific. In particular, the VL and VH Domains maybe selected such that a multivalent diabody may comprise two binding sites for a first epitope and two binding sites for a second epitope, or three binding sites for a first epitope and one binding site for a second epitope, or two binding sites for a first epitope, one binding site for a second epitope and one binding site for a third epitope (as depicted in FIG. 5). The general structure of the polypeptide chains of representative five-chain Fc Region-containing diabodies of invention is provided in Table 3:

TABLE 3

| | | |
|---|---|---|
| Bispecific | 2^{nd} Chain | NH₂-VL1-CL-COOH |
| (2 × 2) | 1^{st} Chain | NH₂-VH1-CH1—CH2—CH3—COOH |
| | 3^{rd} Chain | NH₂-VH1-CH1—CH2—CH3-VL2-VH2-HPD- |
| | | COOH |
| | 5^{nd} Chain | NH₂-VL1-CL—COOH |
| | 4^{th} Chain | NH₂-VL2-VH2-HPD-COOH |
| Bispecific | 2^{nd} Chain | NH₂-VL1-CL-COOH |
| (3 × 1) | 1^{st} Chain | NH₂-VH1-CH1—CH2—CH3—COOH |
| | 3^{rd} Chain | NH₂-VH1-CH1—CH2—CH3-VL1-VH2-HPD- |
| | | COOH |
| | 5^{nd} Chain | NH₂-VL1-CL-COOH |
| | 4^{th} Chain | NH₂-VL2-VH1-HPD-COOH |
| Trispecific | 2^{nd} Chain | NH₂-VL1-CL-COOH |
| (2 × 1 × 1) | 1^{st} Chain | NH₂-VH1-CH1—CH2—CH3—COOH |
| | 3^{rd} Chain | NH₂-VH1-CH1—CH2—CH3-VL2-VH3-HPD- |
| | | COOH |
| | 5^{nd} Chain | NH₂-VL1-CL-COOH |
| | 4^{th} Chain | NH₂-VL3-VH2-HPD-COOH |

HPD = Heterodimer-Promoting Domain

In a specific embodiment, diabodies of the present invention are bispecific, tetravalent (i.e., possess four epitope-binding sites), Fc-containing diabodies that are composed of five total polypeptide chains having two epitope-binding sites immunospecific for ROR1 (which may be capable of binding to the same epitope of ROR1 or to different epitopes of ROR1), and two epitope-binding sites specific for a second molecule (which may be capable of binding to the same epitope of the second molecule or to different epitopes of the second molecule). In another embodiment, the bispecific, tetravalent, Fc-containing diabodies of the invention comprise three epitope-binding sites immunospecific for ROR1 (which may be capable of binding to the same epitope of ROR1 or to two or three different epitopes of ROR1), and one epitope-binding site specific for a second molecule. In another embodiment, the bispecific, tetravalent, Fc-containing diabodies of the invention comprise one epitope-binding site immunospecific for ROR1, and three epitope-binding sites specific for a second molecule (which may be capable of binding to the same epitope of the second molecule or to two or three different epitopes of the second molecule). As provided above, the VL and VH domains may be selected to permit trispecific binding. Accordingly, the invention also encompasses trispecific, tetravalent, Fc-containing diabodies. The trispecific, tetravalent, Fc-containing diabodies of the invention comprise two epitope-binding sites immunospecific for ROR1, one epitope-binding site immunospecific for a second molecule, and one epitope-binding site immunospecific for a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the second molecule is CD3 and the third molecule is CD8.

D. Trivalent Binding Molecules Containing Fc Regions

A further embodiment of the present invention relates to trivalent binding molecules comprising an Fc Region capable of simultaneously binding a first epitope, a second epitope and a third epitope, wherein at least one of such epitopes is not identical to another. Such trivalent binding molecules comprise three epitope-binding sites, two of which are Diabody-Type Binding Domains, which provide binding Site A and binding Site B, and one of which is a Fab-Type Binding Domain, or an scFv-Type Binding Domain, which provides binding Site C (see, e.g., FIGS. 6A-6F, and PCT Application No: PCT/US15/33081; and PCT/US15/33076). Such trivalent binding molecules thus comprise “VL1”/“VH1” domains that are capable of binding to the first epitope and “VL2”/“VH2” domains that are capable of binding to the second epitope and “VL3” and “VH3” domains that are capable of binding to the “third” epitope of such trivalent binding molecule. A “Diabody-Type Binding Domain” is the type of epitope-binding site present in a diabody, and especially, a DART® diabody, as described above. Each of a “Fab-Type Binding Domain” and an “scFv-Type Binding Domain” are epitope-binding sites that are formed by the interaction of the VL Domain of an immunoglobulin light chain and a complementing VH Domain of an immunoglobulin heavy chain. Fab-Type Binding Domains differ from Diabody-Type Binding Domains in that the two polypeptide chains that form a Fab-Type Binding Domain comprise only a single epitope-binding site, whereas the two polypeptide chains that form a Diabody-Type Binding Domain comprise at least two epitope-binding sites. Similarly, scFv-Type Binding Domains also differ from Diabody-Type Binding Domains in that they comprise only a single epitope-binding site. Thus, as used herein Fab-Type, and scFv-Type Binding Domains are distinct from Diabody-Type Binding Domains.

Typically, the trivalent binding molecules of the present invention will comprise four different polypeptide chains (see FIGS. 6A-6B), however, the molecules may comprise fewer or greater numbers of polypeptide chains, for example by fusing such polypeptide chains to one another (e.g., via a peptide bond) or by dividing such polypeptide chains to form additional polypeptide chains, or by associating fewer or additional polypeptide chains via disulfide bonds. FIGS. 6C-6F illustrate this aspect of the present invention by schematically depicting such molecules having three polypeptide chains. As provided in FIGS. 6A-6F, the trivalent binding molecules of the present invention may have alternative orientations in which the Diabody-Type Binding Domains are N-terminal (FIGS. 6A, 6C and 6D) or C-terminal (FIGS. 6B, 6E and 6F) to an Fc Region.

In certain embodiments, the first polypeptide chain of such trivalent binding molecules of the present invention contains: (i) a VL1-containing Domain, (ii) a VH2-containing Domain, (iii) a Heterodimer-Promoting Domain, and (iv) a Domain containing a CH2-CH3 sequence. The VL1 and VL2 Domains are located N-terminal or C-terminal to the CH2-CH3-containing domain as presented in Table 4 (also see, FIGS. 6A and 6B). The second polypeptide chain of such embodiments contains: (i) a VL2-containing Domain, (ii) a VH1-containing Domain, and (iii) a Heterodimer-Promoting Domain. The third polypeptide chain of such embodiments contains: (i) a VH3-containing Domain, (ii) a CH1-containing Domain and (iii) a Domain containing a CH2-CH3 sequence. The third polypeptide chain may be the heavy chain of an antibody that contains a VH3 and a heavy chain constant region, or a polypeptide that contains such domains. The fourth polypeptide of such embodiments contains: (i) a VL3-containing Domain and (ii) a CL-containing Domain. The fourth polypeptide chains may be a light chain of an antibody that contains a VL3 complementary to the VH3 of the third polypeptide chain, or a polypeptide that contains such domains. The third or fourth polypeptide chains may be isolated from naturally occurring antibodies. Alternatively, they may be constructed recombinantly, synthetically or by other means.

The Light Chain Variable Domain of the first and second polypeptide chains are separated from the Heavy Chain Variable Domains of such polypeptide chains by an intervening spacer peptide having a length that is too short to permit their VL1/VH2 (or their VL2/VH1) domains to associate together to form epitope-binding site capable of binding to either the first or second epitope. A preferred intervening spacer peptide (Linker 1) for this purpose has the sequence (SEQ ID NO:33): GGGSGGGG. Other Domains of the trivalent binding molecules may be separated by one or more intervening spacer peptides (Linkers), optionally comprising a cysteine residue. In particular, as provided above, such Linkers will typically be incorporated between Variable Domains (i.e., VH or VL) and peptide Heterodimer-Promoting Domains (e.g., an E-coil or K-coil) and between such peptide Heterodimer-Promoting Domains (e.g., an E-coil or K-coil) and CH2-CH3 Domains. Exemplary linkers useful for the generation of trivalent binding molecules are provided above and are also provided in PCT Application Nos: PCT/US15/33081; and PCT/US15/33076. Thus, the first and second polypeptide chains of such trivalent binding molecules associate together to form a VL1/VH1 binding site capable of binding a first epitope, as well as a VL2/VH2 binding site that is capable of binding to a second epitope. The third and fourth polypeptide chains of such trivalent binding molecules associate together to form a VL3/VH3 binding site that is capable of binding to a third epitope.

As described above, the trivalent binding molecules of the present invention may comprise three polypeptides. Trivalent binding molecules comprising three polypeptide chains may be obtained by linking the domains of the fourth polypeptide N-terminal to the VH3-containing Domain of the third polypeptide (e.g., using an intervening spacer peptide (Linker 4)). Alternatively, a third polypeptide chain of a trivalent binding molecule of the invention containing the following domains is utilized: (i) a VL3-containing Domain, (ii) a VH3-containing Domain, and (iii) a Domain containing a CH2-CH3 sequence, wherein the VL3 and VH3 are spaced apart from one another by an intervening spacer peptide that is sufficiently long (at least 9 or more amino acid residues) so as to allow the association of these domains to form an epitope-binding site. One preferred intervening spacer peptide for this purpose has the sequence: GGGGSGGGGSGGGGS (SEQ ID NO:64).

It will be understood that the VL1/VH1, VL2/VH2, and VL3/VH3 Domains of such trivalent binding molecules may be different so as to permit binding that is monospecific, bispecific or trispecific. In particular, the VL and VH Domains may be selected such that a trivalent binding molecule comprises two binding sites for a first epitope and one binding sites for a second epitope, or one binding site for a first epitope and two binding sites for a second epitope, or one binding site for a first epitope, one binding site for a second epitope and one binding site for a third epitope.

However, as provided herein, these domains are preferably selected so as to bind an epitope of ROR1, an epitope of second molecule, and an epitope of a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the third molecule is CD8.

The general structure of the polypeptide chains of representative trivalent binding molecules of invention is provided in FIGS. 6A-6F and in Table 4:

TABLE 4

| | | |
|---|---|---|
| Four Chain | 2^{nd} Chain | NH₂-VL2-VH1-HPD-COOH |
| 1^{st} | 1^{st} Chain | NH₂-VL1-VH2-HPD-CH2—CH3—COOH |
| Orientation | 3^{rd} Chain | NH₂-VH3-CH1—CH2—CH3—COOH |
| | 2^{nd} Chain | NH₂-VL3-CL-COOH |
| Four Chain | 2^{nd} Chain | NH₂-VL2-VH1-HPD-COOH |
| 2^{nd} | 1^{st} Chain | NH₂—CH2—CH3-VL1-VH2-HPD-COOH |
| Orientation | 3^{rd} Chain | NH₂-VH3-CH1—CH2—CH3—COOH |
| | 2^{nd} Chain | NH₂-VL3-CL-COOH |
| Three Chain | 2^{nd} Chain | NH₂-VL2-VH1-HPD-COOH |
| 1^{st} | 1^{st} Chain | NH₂-VL1-VH2-HPD-CH2—CH3—COOH |
| Orientation | 3^{rd} Chain | NH₂-VL3-VH3-HPD-CH2—CH3—COOH |
| Three Chain | 2^{nd} Chain | NH₂-VL2-VH1-HPD-COOH |
| 2^{nd} | 1^{st} Chain | NH₂—CH2—CH3-VL1-VH2-HPD-COOH |
| Orientation | 3^{rd} Chain | NH₂-VL3-VH3-HPD-CH2—CH3—COOH |

HPD = Heterodimer-Promoting Domain

One embodiment of the present invention relates to trivalent binding molecules that comprise two epitope-binding sites for ROR1 and one epitope-binding site for a second molecule. The two epitope-binding sites for ROR1 may bind the same epitope or different epitopes. Another embodiment of the present invention relates to trivalent binding molecules that comprise, one epitope-binding site for ROR1 and two epitope-binding sites for a second molecule. The two epitope-binding sites for the second molecule may bind the same epitope or different epitopes of the second molecule. A further embodiment of the present invention relates to trispecific trivalent binding molecules that comprise, one epitope-binding site for ROR1, one epitope-binding site for a second molecule, and one epitope-binding site for a third molecule. In certain embodiments, the second molecule is a molecule (e.g., CD2, CD3, CD8, CD16, T-Cell Receptor (TCR), NKG2D, etc.) present on the surface of an effector cell, such as a T lymphocyte, a natural killer (NK) cell or other mononuclear cell. In certain embodiments, the second molecule is CD3 and the third molecule is CD8. As provided above, such trivalent binding molecules may comprise three, four, five, or more polypeptide chains.

### VII. Constant Domains and Variant Fc Regions

Provided herein are antibody “Constant Domains” useful in the generation of the ROR1-binding molecules (e.g., antibodies, diabodies, trivalent binding molecules, etc.) of the invention.

A preferred CL Domain is a human IgG CL Kappa Domain. The amino acid sequence of an exemplary human CL Kappa Domain is (SEQ ID NO:65):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG CL Lambda Domain. The amino acid sequence of an exemplary human CL Lambda Domain is (SEQ ID NO:66):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

As provided herein, the ROR1-binding molecules of the invention may comprise an Fc Region. The Fc Region of such molecules of the invention may be of any isotype (e.g., IgG1, IgG2, IgG3, or IgG4). The ROR1-binding molecules of the invention may further comprise a CH1 Domain and/or a Hinge Region. When present, the CH1 Domain and/or Hinge Region may be of any isotype (e.g., IgG1, IgG2, IgG3, or IgG4), and is preferably of the same isotype as the desired Fc Region.

An exemplary CH1 Domain is a human IgG1 CH1 Domain. The amino acid sequence of an exemplary human IgG1 CH1 Domain is (SEQ ID NO:67):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRV

An exemplary CH1 Domain is a human IgG2 CH1 Domain. The amino acid sequence of an exemplary human IgG2 CH1 Domain is (SEQ ID NO:68):

ASTKGPSVFP LAPCSRSTSE STAALGCLVK DYFPEPVTVS
WNSGALTSGV HTFPAVLQSS GLYSLSSVVT VPSSNFGTQT
YTCNVDHKPS NTKVDKTV

An exemplary CH1 Domain is a human IgG3 CH1 Domain. The amino acid sequence of an exemplary human IgG3 CH1 Domain is (SEQ ID NO:117):

ASTKGPSVFP LAPCSRSTSG GTAALGCLVK DYFPEPVTVS
WNSGALTSGV HTFPAVLQSS GLYSLSSVVT VPSSSLGTQT
YTCNVNHKPS NTKVDKRV

An exemplary CH1 Domain is a human IgG4 CH1 Domain. The amino acid sequence of an exemplary human IgG4 CH1 Domain is (SEQ ID NO:69):

ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRV

One exemplary Hinge Region is a human IgG1 Hinge Region. The amino acid sequence of an exemplary human IgG1 Hinge Region is (SEQ ID NO:60):

EPKSCDKTHTCPPCP.

Another exemplary Hinge Region is a human IgG2 Hinge Region. The amino acid sequence of an exemplary human IgG2 Hinge Region is (SEQ ID NO:61):

ERKCCVECPPCP.

Another exemplary Hinge Region is a human IgG3 Hinge Region. The amino acid sequence of an exemplary human IgG3 Hinge Region is (SEQ ID NO:116):

ELKTPLGDTTHTCPRCPEPKSCDTPPPCPRCPEPKSCDTPPPCPRCPEPK
SCDTPPPCPRCP.

Another exemplary Hinge Region is a human IgG4 Hinge Region. The amino acid sequence of an exemplary human IgG4 Hinge Region is (SEQ ID NO:62): ESKYGPPCPSCP. As described herein, an IgG4 Hinge Region may comprise a stabilizing mutation such as the S228P substitution. The amino acid sequence of an exemplary stabilized IgG4 Hinge Region is (SEQ ID NO:63): ESKYGPPCPPCP.

The Fc Region of the Fc Region-containing molecules (e.g., antibodies, diabodies, trivalent binding molecules, etc.) of the present invention may be either a complete Fc Region (e.g., a complete IgG Fc Region) or only a fragment of an Fc Region. Optionally, the Fc Region of the Fc Region-containing molecules of the present invention lacks the C-terminal lysine amino acid residue.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. All of these interactions are initiated through the binding of the Fc Region of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors; FcγRIIB (CD32B) is an inhibiting (i.e., immune system dampening) receptor. In addition, interaction with the neonatal Fc Receptor (FcRn) mediates the recycling of IgG molecules from the endosome to the cell surface and release into the blood. The amino acid sequence of exemplary wild-type IgG1 (SEQ ID NO:1), IgG2 (SEQ ID NO:2), IgG3 (SEQ ID NO:3), and IgG4 (SEQ ID NO:4) are presented above.

Modification of the Fc Region may lead to an altered phenotype, for example altered serum half-life, altered stability, altered susceptibility to cellular enzymes or altered effector function. It may therefore be desirable to modify an Fc Region-containing ROR1-binding molecule of the present invention with respect to effector function, for example, so as to enhance the effectiveness of such molecule in treating cancer. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor-specific B cells with low levels of FcγRIIB (e.g., non-Hodgkins lymphoma, CLL, and Burkitt' s lymphoma). Molecules of the invention possessing such conferred or altered effector function activity are useful for the treatment and/or prevention of a disease, disorder or infection in which an enhanced efficacy of effector function activity is desired.

Accordingly, in certain embodiments, the Fc Region of the Fc Region-containing molecules of the present invention may be an engineered variant Fc Region. Although the Fc Region of the bispecific Fc Region-containing molecules of the present invention may possess the ability to bind to one or more Fc receptors (e.g., FcγR(s)), more preferably such variant Fc Region have altered binding to FcγRIA (CD64), FcγRIIA (CD32A), FcγRIIB (CD32B), FcγRIIIA (CD16a) or FcγRIIIB (CD16b) (relative to the binding exhibited by a wild-type Fc Region), e.g., will have enhanced binding to an activating receptor and/or will have substantially reduced or no ability to bind to inhibitory receptor(s). Thus, the Fc Region of the Fc Region-containing molecules of the present invention may include some or all of the CH2 Domain and/or some or all of the CH3 Domain of a complete Fc Region, or may comprise a variant CH2 and/or a variant CH3 sequence (that may include, for example, one or more insertions and/or one or more deletions with respect to the CH2 or CH3 domains of a complete Fc Region). Such Fc Regions may comprise non-Fc polypeptide portions, or may comprise portions of non-naturally complete Fc Regions, or may comprise non-naturally occurring orientations of CH2 and/or CH3 Domains (such as, for example, two CH2 domains or two CH3 domains, or in the N-terminal to C-terminal direction, a CH3 Domain linked to a CH2 Domain, etc.).

Fc Region modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Table 5 lists exemplary single, double, triple, quadruple and quintuple substitutions (numbering and substitutions are relative to the amino acid sequence of SEQ ID NO:1) of exemplary modification that increase binding to activating receptors and/or reduce binding to inhibitory receptors.

TABLE 5
Variations of Preferred Activating Fc Regions
Single-Site Variations

| | | | |
|---|---|---|---|
| F243L | R292G | D270E | R292P |
| Y300L | P396L | | |

Double-Site Variation

| | | | |
|---|---|---|---|
| F243L and R292P | F243L and Y300L | F243L and P396L | R292P and Y300L |
| D270E and P396L | R292P and V3051 | P396L and Q419H | P247L and N421K |
| R292P and P396L | Y300L and P396L | R255L and P396L | R292P and P3051 |

K392T and P396L

Triple-Site Variations

| | |
|---|---|
| F243L, P247L and N421K | P247L, D270E and N421K |
| F243L, R292P and Y300L | R255L, D270E and P396L |
| F243L, R292P and V305I | D270E, G316D and R416G |
| F243L, R292P and P396L | D270E, K392T and P396L |
| F243L, Y300L and P396L | D270E, P396L and Q419H |
| V284M, R292L and K370N | R292P, Y300L and P396L |

Quadruple-Site Variations

| | |
|---|---|
| L234F, F243L, R292P and Y300L | F243L, P247L, D270E and N421K |
| L234F, F243L, R292P and Y300L | F243L, R255L, D270E and P396L |
| L235I, F243L, R292P and Y300L | F243L, D270E, G316D and R416G |
| L235Q, F243L, R292P and Y300L | F243L, D270E, K392T and P396L |
| P247L, D270E, Y300L and N421K | F243L, R292P, Y300L, and P396L |
| R255L, D270E, R292G and P396L | F243L, R292P, V305I and P396L |
| R255L, D270E, Y300L and P396L | F243L, D270E, P396L and Q419H |
| D270E, G316D, P396L and R416G | |

Quintuple-Site Variations

| | |
|---|---|
| L235V, F243L, R292P, Y300L and P396L | F243L, R292P, V305I, Y300L and P396L |
| L235P, F243L, R292P, Y300L and P396L | |

Exemplary variants of human IgG1 Fc Regions with reduced binding to CD32B and/or increased binding to CD16A contain F243L, R292P, Y300L, V305I or P296L substitutions. These amino acid substitutions may be present in a human IgG1 Fc Region in any combination. In one embodiment, the variant human IgG1 Fc Region contains a F243L, R292P and Y300L substitution. In another embodiment, the variant human IgG1 Fc Region contains a F243L, R292P, Y300L, V305I and P296L substitution.

In certain embodiments, it is preferred for the Fc Regions of ROR1-binding molecules of the present invention to exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type IgG1 Fc Region (SEQ ID NO:1). In a specific embodiment, the ROR1-binding molecules of the present invention comprise an IgG Fc Region that exhibits reduced ADCC effector function. In a preferred embodiment the CH2-CH3 Domains of such ROR1-binding molecules include any 1, 2, 3, or 4 of the substitutions: L234A, L235A, D265A, N297Q, and N297G. In another embodiment, the CH2-CH3 Domains contain an N297Q substitution, an N297G substitution, L234A and L235A substitutions or a D265A substitution, as these mutations abolish FcR binding. Alternatively, a CH2-CH3 Domain of a naturally occurring Fc region that inherently exhibits decreased (or substantially no) binding to FcγRIIIA (CD16a) and/or reduced effector function (relative to the binding and effector function exhibited by the wild-type IgG1 Fc Region (SEQ ID NO:1)) is utilized. In a specific embodiment, the ROR1-binding molecules of the present invention comprise an IgG2 Fc Region (SEQ ID NO:2) or an IgG4 Fc Region (SEQ ID:NO:4). When an IgG4 Fc Region is utilized, the instant invention also encompasses the introduction of a stabilizing mutation, such as the Hinge Region S228P substitution described above (see, e.g., SEQ ID NO:63). Since the N297G, N297Q, L234A, L235A and D265A substitutions abolish effector function, in circumstances in which effector function is desired, these substitutions would preferably not be employed.

A preferred IgG1 sequence for the CH2 and CH3 Domains of the Fc Region-containing molecules of the present invention having reduced or abolished effector function will comprise the substitutions L234A/L235A (SEQ ID NO:70):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

The serum half-life of proteins comprising Fc Regions may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered.

In some embodiments, the ROR1-binding molecules of the present invention comprise a variant Fc Region, wherein said variant Fc Region comprises at least one amino acid modification relative to a wild-type Fc Region, such that said molecule has an increased half-life (relative to a molecule comprising a wild-type Fc Region). In some embodiments, the ROR1-binding molecules of the present invention comprise a variant IgG Fc Region, wherein said variant Fc Region comprises a half-live extending amino acid substitution at one or more positions selected from the group consisting of 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436. Numerous mutations capable of increasing the half-life of an Fc Region-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and PCT Publication Nos. WO 98/23289; WO 2009/058492; and WO 2010/033279, which are herein incorporated by reference in their entireties. ROR1-binding molecules with enhanced half-life also include those possessing variant Fc Regions comprising substitutions at two or more of Fc Region residues 250, 252, 254, 256, 257, 288, 307, 308, 309, 311, 378, 428, 433, 434, 435 and 436. In particular, two or more substitutions selected from: T250Q, M252Y, S254T, T256E, K288D, T307Q, V308P, A378V, M428L, N434A, H435K, and Y436I.

In a specific embodiment, a ROR1-binding molecule of the present invention possesses a variant IgG Fc Region comprising the substitutions:

(A) M252Y, S254T and T256E;

(B) M252Y and S254T;

(C) M252Y and T256E;

(D) T250Q and M428L;

(E) T307Q and N434A;

(F) A378V and N434A;

(G) N434A and Y436I;

(H) V308P and N434A; or

(I) K288D and H435K.

In a preferred embodiment, a ROR1-binding molecule of the present invention possesses a variant IgG Fc Region comprising any 1, 2, or 3 of the substitutions: M252Y, S254T and T256E. The invention further encompasses ROR1-binding molecules possessing variant Fc Regions comprising:

(A) one or more mutations which alter effector function and/or FcγR; and

(B) one or more mutations which extend serum half-life.

For certain antibodies, diabodies and trivalent binding molecules whose Fc Region-containing first and third polypeptide chains are not identical, it is desirable to reduce or prevent homodimerization from occurring between the CH2-CH3 Domains of two first polypeptide chains or between the CH2-CH3 Domains of two third polypeptide chains. The CH2 and/or CH3 Domains of such polypeptide chains need not be identical in sequence, and advantageously are modified to foster complexing between the two polypeptide chains. For example, an amino acid substitution (preferably a substitution with an amino acid comprising a bulky side group forming a “knob”, e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., “the hole” (e.g., a substitution with glycine). Such sets of mutations can be engineered into any pair of polypeptides comprising CH2-CH3 Domains that forms an Fc Region to foster heterodimerization. Methods of protein engineering to favor heterodimerization over homodimerization are well known in the art, in particular with respect to the engineering of immunoglobulin-like molecules, and are encompassed herein (see e.g., Ridgway et al. (1996) “‘Knobs-Into-Holes’ Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

A preferred knob is created by modifying an IgG Fc Region to contain the modification T366W. A preferred hole is created by modifying an IgG Fc Region to contain the modification T366S, L368A and Y407V. To aid in purifying the hole-bearing third polypeptide chain homodimer from the final bispecific heterodimeric Fc Region-containing molecule, the protein A binding site of the hole-bearing CH2 and CH3 Domains of the third polypeptide chain is preferably mutated by amino acid substitution at position 435 (H435R). Thus, the hole-bearing third polypeptide chain homodimer will not bind to protein A, whereas the bispecific heterodimer will retain its ability to bind protein A via the protein A binding site on the first polypeptide chain. In an alternative embodiment, the hole-bearing third polypeptide chain may incorporate amino acid substitutions at positions 434 and 435 (N434A/N435K).

A preferred IgG amino acid sequence for the CH2 and CH3 Domains of the first polypeptide chain of an Fc Region-containing molecule of the present invention will have the “knob-bearing” sequence (SEQ ID NO:71):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is a lysine (K) or is absent.

A preferred IgG amino acid sequence for the CH2 and CH3 Domains of the second polypeptide chain of an Fc Region-containing molecule of the present invention having two polypeptide chains (or the third polypeptide chain of an Fc Region-containing molecule having three, four, or five polypeptide chains) will have the “hole-bearing” sequence (SEQ ID NO:72):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is a lysine (K) or is absent.

As will be noted, the CH2-CH3 Domains of SEQ ID NO:71, and SEQ ID NO:72 include a substitution at position 234 with alanine and 235 with alanine, and thus form an Fc Region exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc Region (SEQ ID NO:1). The invention also encompasses such CH2-CH3 Domains, which comprise the wild-type alanine residues, alternative and/or additional substitutions which modify effector function and/or FγR binding activity of the Fc region. The invention also encompasses such CH2-CH3 Domains, which further comprise one or more half-live extending amino acid substitutions. In particular, the invention encompasses such hole-bearing and such knob-bearing CH2-CH3 Domains which further comprise the M252Y/S254T/T256E.

It is preferred that the first polypeptide chain will have a “knob-bearing” CH2-CH3 sequence, such as that of SEQ ID NO:71. However, as will be recognized, a “hole-bearing” CH2-CH3 Domain (e.g., SEQ ID NO:72) could be employed in the first polypeptide chain, in which case, a “knob-bearing” CH2-CH3 Domain (e.g., SEQ ID NO:71) would be employed in the second polypeptide chain of an Fc Region-containing molecule of the present invention having two polypeptide chains (or in the third polypeptide chain of an Fc Region-containing molecule having three, four, or five polypeptide chains).

In other embodiments, the invention encompasses ROR1-binding molecules comprising CH2 and/or CH3 Domains that have been engineered to favor heterodimerization over homodimerization using mutations known in the art, such as those disclosed in PCT Publication No. WO 2007/110205; WO 2011/143545; WO 2012/058768; WO 2013/06867, all of which are incorporated herein by reference in their entirety.

### VIII. Effector Cell Binding Capabilities

As provided herein, the ROR1-binding molecules of the invention can be engineered to comprise a combination of epitope-binding sites that recognize a set of antigens unique to a target cell or tissue type. In particular, the present invention relates to multispecific ROR1-binding molecules that are capable of binding to an epitope of ROR1 and an epitope of a molecule present on the surface of an effector cell, such as a T lymphocyte, a natural killer (NK) cell or other mononuclear cell. For example, the ROR1-binding molecules of the present invention may be construction to comprise an epitope-binding site that immunospecifically binds CD2, CD3, CD8, CD16, T-Cell Receptor (TCR), or NKG2D. The invention also relates to trispecific ROR1-binding molecules that are capable of binding to an epitope of CD3 and an epitope of CD8 (see, e.g., PCT Publication No. WO 2015/026894).

A. CD2 Binding Capabilities

In one embodiment, the bispecific, trispecific or multispecific ROR1-binding molecules of the invention are capable of binding to an epitope of ROR1 and an epitope of CD2. CD2 is a cell adhesion molecule found on the surface of T-cells and natural killer (NK) cells. CD2 enhances NK cell cytotoxicity, possibly as a promoter of NK cell nanotube formation (Mace, E. M. et al. (2014) “Cell Biological Steps and Checkpoints in Accessing NK Cell Cytotoxicity,” Immunol. Cell. Biol. 92(3):245-255; Comerci, C. J. et al. (2012) “CD2 Promotes Human Natural Killer Cell Membrane Nanotube Formation,” PLoS One 7(10):e47664:1-12). Molecules that specifically bind CD2 include the anti-CD2 antibody “Lo-CD2a.”

The amino acid sequence of the VL Domain of Lo-CD2a (ATCC Accession No: 11423; SEQ ID NO:73) is shown below (CDR_{L} residues are shown underlined):

DVVLTQTPPT LLATIGQSVS ISCRSSQSLL HSSGNTYLNW
LLQRTGQSPQ PLIYLVSKLE SGVPNRFSGS GSGTDFTLKI
SGVEAEDLGV YYCMQFTHYP YTFGAGTKLE LK

The amino acid sequence of the VH Domain of Lo-CD2a (ATCC Accession No: 11423); SEQ ID NO:74) is shown below (CDR_{H} residues are shown underlined):

EVQLQQSGPE LQRPGASVKL SCKASGYIFT EYYMYWVKQR
PKQGLELVGR IDPEDGSIDY VEKFKKKATL TADTSSNTAY
MQLSSLTSED TATYFCARGK FNYRFAYWGQ GTLVTVSS

B. CD3 Binding Capabilities

In one embodiment, the bispecific, trispecific or multispecific ROR1-binding molecules of the invention are capable of binding to an epitope of ROR1 and an epitope of CD3. CD3 is a T-cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD36 δ chain, and two CD3ε chains. These chains associate with a molecule known as the T-Cell Receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T-cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139). Molecules that specifically binds CD3 include the anti-CD3 antibodies “CD3 mAb 1” and “OKT3.” The anti-CD3 antibody CD3 mAb 1 is capable of binding non-human primates (e.g., cynomolgus monkey).

The amino acid sequence of the VL Domain of CD3 mAb 1 (SEQ ID NO:75) is shown below (CDR_{L} residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The amino acid sequence of the VH Domain of CD3 mAb 1 (SEQ ID NO:76) is shown below (CDR_{H} residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

As discussed below, in order to illustrate the present invention, bispecific ROR1×CD3-binding molecules were produced. In some of the ROR1×CD3 constructs, a variant of CD3 mAb 1 was employed. The variant “CD3 mAb 1 (D65G),” comprises a the VL Domain of CD3 mAb 1 (SEQ ID NO:75) and a VH CD3 mAb 1 Domain having a D65G substitution (Kabat position 65, corresponding to residue 68 of SEQ ID NO:77).

The amino acid sequence of the VH of CD3 mAb 1 (D65G) (SEQ ID NO:77) is shown below (CDR_{H} residues are shown underlined, the substituted position (D65G) is shown in double underline):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSS

Alternatively, an affinity variant of CD3 mAb 1 may be incorporated. Variants include a low affinity variant designated “CD3 mAb 1 Low” and a variant having a faster off rate designated “CD3 mAb 1 Fast.” The VL Domain of CD mAb 1 (SEQ ID NO:75) is common to CD3 mAb 1 Low and CD3 mAb 1 Fast and is provided above. The amino acid sequences of the VH Domains of each of CD3 mAb 1 Low and CD3 mAb 1 Fast are provided below.

The amino acid sequence of the Variable Heavy Chain Domain of anti-human CD3 mAb 1 Low (SEQ ID NO:78) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVT WFAYWGQGTL
VTVSS

The amino acid sequence of the Variable Heavy Chain Domain of anti-human CD3 mAb 1 Fast (SEQ ID NO:79) is shown below (CDR_{H} residues are shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HKNFGNSYVT WFAYWGQGTL
VTVSS

Another anti-CD3 antibody, which may be utilized is antibody Muromonab-CD3 “OKT3” (Xu et al. (2000) “In Vitro Characterization Of Five Humanized OKT3 Effector Function Variant Antibodies,” Cell. Immunol. 200:16-26); Norman, D. J. (1995) “Mechanisms Of Action And Overview Of OKT3,” Ther. Drug Monit. 17(6):615-620; Canafax, D. M. et al. (1987) “Monoclonal Antilymphocyte Antibody (OKT3) Treatment Of Acute Renal Allograft Rejection,” Pharmacotherapy 7(4):121-124; Swinnen, L. J. et al. (1993) “OKT3 Monoclonal Antibodies Induce Interleukin-6 And Interleukin-10: A Possible Cause Of Lymphoproliferative Disorders Associated With Transplantation,” Curr. Opin. Nephrol. Hypertens. 2(4):670-678).

The amino acid sequence of the VL Domain of OKT3 (SEQ ID NO:80) is shown below (CDR_{L} residues are shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

The amino acid sequence of the VH Domain of OKT3 (SEQ ID NO:81) is shown below (CDR_{H} residues are shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSS

Additional anti-CD3 antibodies that may be utilized include but are not limited to those described in PCT Publication Nos. WO 2008/119566; and WO 2005/118635.

C. CD8 Binding Capabilities

In one embodiment, the bispecific, trispecific or multispecific ROR1-binding molecules of the invention are capable of binding to an epitope of ROR1 and an epitope of CD8. CD8 is a T-cell co-receptor composed of two distinct chains (Leahy, D. J., (1995) “A Structural View of CD4 and CD8,” FASEB J., 9:17-25) that is expressed on Cytotoxic T-cells. The activation of CD8⁺ T-cells has been found to be mediated through co-stimulatory interactions between an antigen:major histocompability class I (MHC I) molecule complex that is arrayed on the surface of a target cell and a complex of CD8 and the T-cell Receptor, that are arrayed on surface of the CD8⁺ T-cell (Gao, G., and Jakobsen, B., (2000). “Molecular interactions of coreceptor CD8 and MHC class I: the molecular basis for functional coordination with the T-Cell Receptor”. Immunol Today 21: 630-636). Unlike MHC II molecules, which are expressed by only certain immune system cells, MHC I molecules are very widely expressed. Thus, cytotoxic T-cells are capable of binding to a wide variety of cell types. Activated cytotoxic T-cells mediate cell killing through their release of the cytotoxins perforin, granzymes, and granulysin. Antibodies that specifically bind CD8 include the anti-CD8 antibodies “OKT8” and “TRX_{2.}”

The amino acid sequence of the VL Domain of OKT8 (SEQ ID NO:82) is shown below (CDR_{L} residues are shown underlined):

DIVMTQSPAS LAVSLGQRAT ISCRASESVD SYDNSLMHWY
QQKPGQPPKV LIYLASNLES GVPARFSGSG SRTDFTLTID
PVEADDAATY YCQQNNEDPY TFGGGTKLEI KR

The amino acid sequence of the VH Domain of OKT8 (SEQ ID NO:83) is shown below (CDR_{H} residues are shown underlined):

QVQLLESGPE LLKPGASVKM SCKASGYTFT DYNMHWVKQS
HGKSLEWIGY IYPYTGGTGY NQKFKNKATL TVDSSSSTAY
MELRSLTSED SAVYYCARNF RYTYWYFDVW GQGTTVTVSS

The amino acid sequence of the VL Domain of TRX₂ (SEQ ID NO:84) is shown below (CDR_{L} residues are shown underlined):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIK

The amino acid sequence of the VH Domain of TRX₂ (SEQ ID NO:85) is shown below (CDR_{H} residues are shown underlined):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
S

D. CD16 Binding Capabilities

In one embodiment, multispecific ROR1-binding molecules of the invention are capable of binding to an epitope of ROR1 and an epitope of CD16. CD16 is the FcγRIIIA receptor. CD16 is expressed by neutrophils, eosinophils, natural killer (NK) cells, and tissue macrophages that bind aggregated but not monomeric human IgG (Peltz, G. A. et al. (1989) “Human Fc Gamma Rill: Cloning, Expression, And Identification Of The Chromosomal Locus Of Two Fc Receptors For IgG,” Proc. Natl. Acad. Sci. (U.S.A.) 86(3):1013-1017; Bachanova, V. et al. (2014) “NK Cells In Therapy Of Cancer,” Crit. Rev. Oncog. 19(1-2): 133-141; Miller, J. S. (2013) “Therapeutic Applications: Natural Killer Cells In The Clinic,” Hematology Am. Soc. Hematol. Educ. Program. 2013:247-253; Youinou, P. et al. (2002) “Pathogenic Effects Of Anti-Fc Gamma Receptor IIIB (CD16) On Polymorphonuclear Neutrophils In Non-Organ-Specific Autoimmune Diseases,” Autoimmun Rev. 1(1-2):13-19; Peipp, M. et al. (2002) “Bispecific Antibodies Targeting Cancer Cells,” Biochem. Soc. Trans. 30(4):507-511). Molecules that specifically bind CD16 include the anti-CD16 antibodies “3G8” and “A9.” Humanized A9 antibodies are described in PCT Publication WO 03/101485.

The amino acid sequence of the VL Domain of 3G8 (SEQ ID NO:86) is shown below (CDR_{L} residues are shown underlined):

DTVLTQSPAS LAVSLGQRAT ISCKASQSVD FDGDSFMNWY
QQKPGQPPKL LIYTTSNLES GIPARFSASG SGTDFTLNIH
PVEEEDTATY YCQQSNEDPY TFGGGTKLEI K

The amino acid sequence of the VH Domain of 3G8 (SEQ ID NO:87) is shown below (CDR_{H} residues are shown underlined):

QVTLKESGPG ILQPSQTLSL TCSFSGFSLR TSGMGVGWIR
QPSGKGLEWL AHIWWDDDKR YNPALKSRLT ISKDTSSNQV
FLKIASVDTA DTATYYCAQI NPAWFAYWGQ GTLVTVSA

The amino acid sequence of the VL Domain of A9 (SEQ ID NO:88) is shown below (CDR_{L} residues are shown underlined):

DIQAVVTQES ALTTSPGETV TLTCRSNTGT VTTSNYANWV
QEKPDHLFTG LIGHTNNRAP GVPARFSGSL IGDKAALTIT
GAQTEDEAIY FCALWYNNHW VFGGGTKLTVL

The amino acid sequence of the VH Domain of A9 (SEQ ID NO:89) is shown below (CDR_{H} residues are shown underlined):

QVQLQQSGAE LVRPGTSVKI SCKASGYTFT NYWLGWVKQR
PGHGLEWIGD IYPGGGYTNY NEKFKGKATV TADTSSRTAY
VQVRSLTSED SAVYFCARSA SWYFDVWGAR TTVTVSS

Additional anti-CD19 antibodies that may be utilized include but are not limited to those described in PCT Publication Nos. WO 03/101485; and WO 2006/125668.

E. TCR Binding Capabilities

In one embodiment, the bispecific, trispecific or multispecific ROR1-binding molecules of the invention are capable of binding to an epitope of ROR1 and an epitope of the T Cell Receptor (TCR). The T Cell Receptor is natively expressed by CD4+ or CD8+ T cells, and permits such cells to recognize antigenic peptides that are bound and presented by class I or class II MHC proteins of antigen-presenting cells. Recognition of a pMHC (peptide—MHC) complex by a TCR initiates the propagation of a cellular immune response that leads to the production of cytokines and the lysis of the antigen-presenting cell (see, e.g., Armstrong, K. M. et al. (2008) “Conformational Changes And Flexibility In T-Cell Receptor Recognition Of Peptide—MHC Complexes,” Biochem. J. 415(Pt 2):183-196; Willemsen, R. (2008) “Selection Of Human Antibody Fragments Directed Against Tumor T-Cell Epitopes For Adoptive T-Cell Therapy,” Cytometry A. 73(11):1093-1099; Beier, K. C. et al. (2007) “Master Switches Of T-Cell Activation And Differentiation,” Eur. Respir. J. 29:804-812; Mallone, R. et al. (2005) “Targeting T Lymphocytes For Immune Monitoring And Intervention In Autoimmune Diabetes,” Am. J. Ther. 12(6):534-550). CD3 is the receptor that binds to the TCR (Thomas, S. et al. (2010) “Molecular Immunology Lessons From Therapeutic T-Cell Receptor Gene Transfer,” Immunology 129(2):170-177; Guy, C. S. et al. (2009) “Organization Of Proximal Signal Initiation At The TCR: CD3 Complex,” Immunol. Rev. 232(1):7-21; St. Clair, E. W. (Epub 2009 Oct. 12) “Novel Targeted Therapies For Autoimmunity,” Curr. Opin. Immunol. 21(6):648-657; Baeuerle, P. A. et al. (Epub 2009 Jun. 9) “Bispecific T-Cell Engaging Antibodies For Cancer Therapy,” Cancer Res. 69(12):4941-4944; Smith-Garvin, J. E. et al. (2009) “T Cell Activation,” Annu. Rev. Immunol. 27:591-619; Renders, L. et al. (2003) “Engineered CD3 Antibodies For Immunosuppression,” Clin. Exp. Immunol. 133 (3) :307-309).

Molecules that specifically bind to the T Cell Receptor include the anti-TCR antibody “BMA 031” (EP 0403156; Kurrle, R. et al. (1989) “BMA 031—A TCR-Specific Monoclonal Antibody For Clinical Application,” Transplant Proc. 21(1 Pt 1): 1017-1019; Nashan, B. et al. (1987) “Fine Specificity Of A Panel Of Antibodies Against The TCR/CD3 Complex,” Transplant Proc. 19(5):4270-4272; Shearman, C. W. et al. (1991) “Construction, Expression, And Biologic Activity Of Murine/Human Chimeric Antibodies With Specificity For The Human α/β T Cell,” J. Immunol. 146(3):928-935; Shearman, C. W. et al. (1991) “Construction, Expression And Characterization of Humanized Antibodies Directed Against The Human α/β T Cell Receptor,” J. Immunol. 147(12):4366-4373).

The amino acid sequence of the VL Domain of BMA 031 (SEQ ID NO:90) is shown below (CDR_{L} residues are shown underlined):

EIVLTQSPAT LSLSPGERAT LSCSATSSVS YMHWYQQKPG
KAPKRWIYDT SKLASGVPSR FSGSGSGTEF TLTISSLQPE
DFATYYCQQW SSNPLTFGQG TKLEIK

The amino acid sequence of a VH Domain of BMA 031 (SEQ ID NO:91) is shown below (CDR_{H} residues are shown underlined):

QVQLVQSGAE VKKPGASVKV SCKASGYKFT SYVMHWVRQA
PGQGLEWIGY INPYNDVTKY NEKFKGRVTI TADKSTSTAY
LQMNSLRSED TAVHYCARGS YYDYDGFVYW GQGTLVTVSS

F. NKG2D Binding Capabilities

In one embodiment, multispecific ROR1-binding molecules of the invention are capable of binding to an epitope of ROR1 and an epitope of the NKG2D receptor. The NKG2D receptor is expressed on all human (and other mammalian) Natural Killer cells (Bauer, S. et al. (1999) “Activation Of NK Cells And T Cells By NKG2D, A Receptor For Stress-Inducible MICA,” Science 285(5428):727-729; Jamieson, A. M. et al. (2002) “The Role Of The NKG2D Immunoreceptor In Immune Cell Activation And Natural Killing,” Immunity 17(1):19-29) as well as on all CD8⁻ T cells (Groh, V. et al. (2001) “Costimulation Of CD8α/β T Cells By NKG2D Via Engagement By MIC Induced On Virus-Infected Cells,” Nat. Immunol. 2(3):255-260; Jamieson, A. M. et al. (2002) “The Role Of The NKG2D Immunoreceptor In Immune Cell Activation And Natural Killing,” Immunity 17(1):19-29). Such binding ligands, and particularly those which are not expressed on normal cells, include the histocompatibility 60 (H60) molecule, the product of the retinoic acid early inducible gene-1 (RAE-1), and the murine UL16-binding protein like transcript 1 (MULTI) (Raulet D. H. (2003) “Roles Of The NKG2D Immunoreceptor And Its Ligands,” Nature Rev. Immunol. 3:781-790; Coudert, J. D. et al. (2005) “Altered NKG2D Function In NK Cells Induced By Chronic Exposure To Altered NKG2D Ligand-Expressing Tumor Cells,” Blood 106:1711-1717). Molecules that specifically bind to the NKG2D Receptor include the anti-NKG2D antibodies “KYK-1.0” and “KYK-2.0” (Kwong, K Y et al. (2008) “Generation, Affinity Maturation, And Characterization Of A Human Anti-Human NKG2D Monoclonal Antibody With Dual Antagonistic And Agonistic Activity,” J. Mol. Biol. 384:1143-1156; and PCT/US09/54911).

The amino acid sequence of the VL Domain of KYK-1.0 (SEQ ID NO:92) is shown below (CDR_{L} residues are shown underlined):

QPVLTQPSSV SVAPGETARI PCGGDDIETK SVHWYQQKPG
QAPVLVIYDD DDRPSGIPER FFGSNSGNTA TLSISRVEAG
DEADYYCQVW DDNNDEWVFG GGTQLTVL

The amino acid sequence of the VH Domain of KYK-1.0 (SEQ ID NO:93) is shown below (CDR_{H} residues are shown underlined):

EVQLVESGGG VVQPGGSLRL SCAASGFTFS SYGMHWVRQA
PGKGLEWVAF IRYDGSNKYY ADSVKGRFTI SRDNSKNTKY
LQMNSLRAED TAVYYCAKDR FGYYLDYWGQ GTLVTVSS

The amino acid sequence of a VL Domain of KYK-2.0 (SEQ ID NO:94) is shown below (CDR_{L} residues are shown underlined):

QSALTQPASV SGSPGQSITI SCSGSSSNIG NNAVNWYQQL
PGKAPKLLIY YDDLLPSGVS DRFSGSKSGT SAFLAISGLQ
SEDEADYYCA AWDDSLNGPV FGGGTKLTVL

The amino acid sequence of a VH Domain of KYK-2.0 (SEQ ID NO:95) is shown below (CDR_{H} residues are shown underlined):

QVQLVESGGG LVKPGGSLRL SCAASGFTFS SYGMHWVRQA
PGKGLEWVAF IRYDGSNKYY ADSVKGRFTI SRDNSKNTLY
LQMNSLRAED TAVYYCAKDR GLGDGTYFDY WGOGTTVTVS S

### IX. Exemplary Multispecific ROR1-Binding Molecules

A. ROR1×CD3 Bispecific Two Chain Diabodies

As provided herein, thirty-three exemplary bispecific two chain “ROR1×CD3” diabodies having one binding site specific for ROR1 (comprising parental and/or optimized anti-ROR1-VL and anti-ROR1-VH Domains) and one binding site specific for CD3 (comprising the VL and VH Domains of CD3 mAb 1 (D65G)) were generated and characterized. Such diabodies, were consecutively numbered and designated “DART-1” to “DART-33.” The structure of these two-chain bispecific ROR1×CD3 diabodies is detailed below. DART-1 comprises the parental anti-ROR1-VL and anti-ROR1-VL Domains. These exemplary chain ROR1×CD3 bispecific two chain diabodies are intended to illustrate, but in no way limit, the scope of the invention.

The first polypeptide chain of the exemplary ROR1×CD3 bispecific two chain diabodies comprises, in the N-terminal to C-terminal direction: an N-terminus; an anti-ROR1-VL Domain selected from SEQ ID NOs:6 and 10-23; an intervening spacer peptide (Linker 1: GGGSGGGG (SEQ ID NO:33)); the VH Domain of CD3 mAb 1 (D65G) (SEQ ID NO:77); a cysteine-containing intervening spacer peptide (Linker 2: GGCGGG (SEQ ID NO:34)); a Heterodimer-Promoting (K-coil) Domain (KVAALKE-KVAALKE-KVAALKE-KVAALKE (SEQ ID NO:47)); and a C-terminus. The particular anti-ROR1-VL Domain present in each diabody is indicated in Table 7 and the amino acid sequences are provided above.

The second polypeptide chain of the exemplary ROR1×CD3 bispecific two chain diabodies comprises, in the N-terminal to C-terminal direction: an N-terminus; the VL Domain of CD3 mAb 1 (SEQ ID NO:75); an intervening spacer peptide (Linker 1: GGGSGGGG (SEQ ID NO:33)); an anti-ROR1-VH Domain selected from SEQ ID NOs:7 and 24-32; a cysteine-containing intervening spacer peptide (Linker 2: GGCGGG (SEQ ID NO:34)); a Heterodimer-Promoting (E-coil) Domain (EVAALEK-EVAALEK-EVAALEK-EVAALEK (SEQ ID NO:46)); and a C-terminus. The particular anti-ROR1-VH Domain present in each diabody is indicated in Table 7 and the amino acid sequences are provided above.

### DART-25

The amino acid sequence of a representative ROR1×CD3 bispecific two chain diabody, DART-25, is provided. DART-25 comprises the optimized anti-ROR1-VL Domain anti-ROR1-VL(2) and the optimized anti-ROR1-VL Domain anti-ROR1-VH(7). The CD3 binding domains of DART-25 are the VH domain of CD3 mAb 1 (D65G) (SEQ ID NO:77) and the VL domain of CD3 mAb 1 (SEQ ID NO:75). The anti-ROR1 binding domains and anti-CD3 binding domains are separated from one another by an intervening spacer peptide (Linker 1) GGGSGGGG (SEQ ID NO:33).

The amino acid sequence of the first polypeptide chain of DART-25 (SEQ ID NO:96) is shown below (the anti-ROR1-VL(2) is shown in solid underlined; the VH Domain of anti-CD3 mAb 1 (D65G) is shown in dotted underline):

QLVLTQSPSA SASLGSSVKL TCTLSSGHKT DTIDWYQQQP
GKAPRYLMKL EGSGSYNKGS GVPDRFGSGS SSGADWYLTI
SSLQSEDEAD YYCGTDYPGN YLFGGGTQLT VLGGGGSGGG

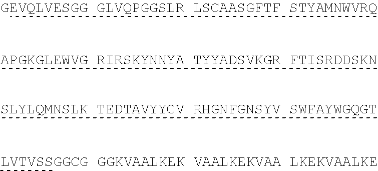

The amino acid sequence of the second polypeptide chain of DART-25 (SEQ ID NO:97) is shown below (the anti-ROR1-VH(7) is shown in solid underlined; the VL domain of CD3 mAb 1 is shown in dotted underline):

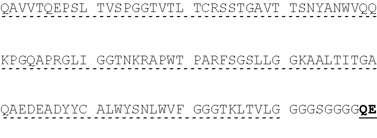

QLVESGGGLV QPGGSLRLSC AASGFTFSDY YMSWVRQAPG
KGLEWVATIY PSSGKTYYAD SVKGRLTISS DNAKDSLYLQ
MNSLRAEDTA VYYCTRDSYA DDAALFDIWG QGTTVTVSSG
GCGGGEVAAL EKEVAALEKE VAALEKEVAA LEK

It will be appreciated in view of the teachings provided herein that different domain orientations, VH Domains, VL Domains, linkers, and/or heterodimer promoting domains, could be utilized to generate alternative ROR1×CD3 bispecific two chain diabodies. For example, different anti-ROR1-VL and/or VH Domains were utilized to generate DART-1 to DART-33 (see, e.g., Table 7). Furthermore, any of the optimized anti-ROR1-VL and/or VH Domains provided herein (preferably, SEQ ID NOs:23 and SEQ ID NOs:31), may be used in place of anti-ROR1-VL(2) and/or anti-ROR1-VH(7) to generate alternative molecules.

B. ROR1×CD3 Bispecific Three Chain Diabodies

As provided herein, four exemplary bispecific three chain “ROR1×CD3” diabodies having one binding site specific for ROR1 (comprising parental and/or optimized anti-ROR1-VL and anti-ROR1-VH Domains) and one binding site specific for CD3 (comprising the VL and VH Domains of CD3 mAb 1 (D65G)) were generated and characterized. The exemplary bispecific three chain diabodies were designated as follows: “DART-A,” which comprises the parental anti-ROR1-VL and anti-ROR1-VH Domains; “DART-B,” which comprises the optimized anti-ROR1-VL(1) and parental anti-ROR1-VH Domains; “DART-C,” which comprises the optimized anti-ROR1-VL(14) and anti-ROR1-VH(7) Domains; and “DART-D,” which comprises the optimized anti-ROR1-VL(14) and anti-ROR1-VH(8) Domains. The structure of these ROR1×CD3 bispecific three chain diabodies is detailed below. These exemplary ROR1×CD3 bispecific three chain diabodies are intended to illustrate, but in no way limit, the scope of the invention.

The first polypeptide chain of the exemplary ROR1×CD3 bispecific three chain diabodies comprises, in the N-terminal to C-terminal direction: an N-terminus; an anti-ROR1-VL Domain (SEQ ID NO:6 for DART-A, SEQ ID NO:10 for DART-B, SEQ ID NO:23 for DART-C, or SEQ ID NO:23 for DART-D); an intervening spacer peptide (Linker 1: GGGSGGGG (SEQ ID NO:33)); the VH Domain of CD3 mAb 1 (D65G) (SEQ ID NO:77); an intervening spacer peptide (Linker 2: ASTKG (SEQ ID NO:38)); a cysteine-containing Heterodimer-Promoting (E-coil) Domain (EVAACEK-EVAALEK-EVAALEK-EVAALEK (SEQ ID NO:48)); an intervening spacer peptide (Linker 3: GGGDKTHTCPPCP (SEQ ID NO:58)); a knob-bearing IgG1 CH2-CH3 Domain (SEQ ID NO:71); and a C-terminus. Encoding polynucleotides for this polypeptide chain may encode the C-terminal lysine residue of SEQ ID NO:71 (i.e., X of SEQ ID NO:71), however, as discussed above, this lysine residue may be post-translationally removed in some expression systems. Accordingly, the invention encompasses such a first polypeptide chain that contains such lysine residue (i.e., SEQ ID NO:71, wherein X is lysine), as well as a first polypeptide chain that lacks such lysine residue (i.e., SEQ ID NO:71, wherein X is absent). The anti-ROR1-VL Domain present in each diabody is indicated in Table 9 and the amino acid sequences are provided below.

The second polypeptide chain of the exemplary ROR1×CD3 bispecific three chain diabodies comprises, in the N-terminal to C-terminal direction: an N-terminus; the VL Domain of CD3 mAb 1 (SEQ ID NO:75); an intervening spacer peptide (Linker 1: GGGSGGGG (SEQ ID NO:33)); an anti-ROR1-VH Domain (SEQ ID NO:7 for DART-A, SEQ ID NO:7 for DART-B, SEQ ID NO:30 for DART-C, or SEQ ID NO:31 for DART-D); an intervening spacer peptide (Linker 2: ASTKG (SEQ ID NO:38)); a cysteine-containing Heterodimer-Promoting (K-coil) Domain (KVAACKE-KVAALKE-KVAALKE-KVAALKE (SEQ ID NO:49)); and a C-terminus. The anti-ROR1-VH Domain present in each diabody is indicated in Table 9 and the amino acid sequences are provided below.

The third polypeptide chain of the exemplary ROR1×CD3 bispecific three chain diabodies comprises, in the N-terminal to C-terminal direction: an N-terminus; a spacer peptide (DKTHTCPPCP (SEQ ID NO:57)); a hole-bearing IgG1 CH2-CH3 Domain (SEQ ID NO:72); and a C-terminus. Encoding polynucleotides for this polypeptide chain may encode the C-terminal lysine residue of SEQ ID NO:72 (i.e., X of SEQ ID NO:72), however, as discussed above, this lysine residue may be post-translationally removed in some expression systems. Accordingly, the invention encompasses such a third polypeptide chain that contains such lysine residue (i.e., SEQ ID NO:72, wherein X is lysine), as well as a third polypeptide chain that lacks such lysine residue (i.e., SEQ ID NO:72, wherein X is absent). The third polypeptide chain is common to each of the exemplary ROR1×CD3 bispecific three chain diabodies.

### DART-A

Thus, the amino acid sequence of the first polypeptide chain of DART-A (SEQ ID NO:98) is shown below (parental anti-ROR1-VL is underlined):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLGGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of DART-A (SEQ ID NO:99) is shown below (parental anti-ROR1-VH is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWVRQAPG
KGLEWVATIY PSSGKTYYAD SVKGRFTISS DNAKNSLYLQ
MNSLRAEDTA VYYCARDSYA DDAALFDIWG QGTTVTVSSA
STKGKVAACK EKVAALKEKV AALKEKVAAL KE

The amino acid sequence of the third polypeptide chain for DART-A is SEQ ID NO:100:

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

### DART-B

The amino acid sequence of the first polypeptide chains of DART-B is identical to that of DART-A except that a G residue between Kabat positions 63 and 64 is deleted (underlined) (SEQ ID NO:101):

QLVLTQSPSA SASLGSSVKL TCTLSSGHKT DTIDWYQQQP
GKAPRYLMKL EGSGSYNKGS GVPDRF_SGS SSGADRYLTI
SSLQSEDEAD YYCGTDYPGN YLFGGGTQLT VLGGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of DART-B is identical to that of the second polypeptide chain of DART-A (SEQ ID NO:99). The amino acid sequence of the third polypeptide chain of DART-B is identical to that of the third polypeptide chain of DART-A (SEQ ID NO:100).

### DART-C

The amino acid sequence of the first polypeptide chain of DART-C (SEQ ID NO:102) is shown below (anti-ROR1-VL(14) is underlined):

QLVLTQSPSA SASLGSSVKL TCTLSSGHKT DTIDWYQQQP
GKAPRYLMKL EGSGSYNKGS GVPDRFSGSS SGADWYLTIS
SLQSEDEADY YCGTDYPGNY LFGGGTQLTV LGGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSASTKG EVAACEKEVA ALEKEVAALE KEVAALEKGG
GDKTHTCPPC PAPEAAGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSREEMT KNQVSLWCLV KGFYPSDIAV
EWESNGQPEN NYKTTPPVLD SDGSFFLYSK LTVDKSRWQQ
GNVFSCSVMH EALHNHYTQK SLSLSPGK

The amino acid sequence of the second polypeptide chain of DART-C (SEQ ID NO:103) is shown below (anti-ROR1-VH(7) is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWVRQAPG
KGLEWVATIY PSSGKTYYAD SVKGRLTISS DNAKDSLYLQ
MNSLRAEDTA VYYCTRDSYA DDAALFDIWG QGTTVTVSSA
STKGKVAACK EKVAALKEKV AALKEKVAAL KE

The amino acid sequence of the third polypeptide chain of DART-C is identical to that of the third polypeptide chain of DART-A (SEQ ID NO:100).

### DART-D

The amino acid sequence of the first polypeptide chain of DART-D is identical to that of the first polypeptide chain of DART-C (SEQ ID NO:102).

The amino acid sequence of the second polypeptide chain of DART-D (SEQ ID NO:104) is shown below (anti-ROR1-VH(8) is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWIRQAPG
KGLEWVATIY PSSGKTYYAD SAKGRLTISS DNAKDSLYLQ
MNSLRAEDTA VYYCTRDSYA DDAALFDIWG QGTTVTVSSA
STKGKVAACK EKVAALKEKV AALKEKVAAL KE

The amino acid sequence of the third polypeptide chain of DART-D is identical to that of the third polypeptide chain of DART-A (SEQ ID NO:100).

It will be appreciated in view of the teachings provided herein that different domain orientations, VH Domains, VL Domains, linkers, heterodimer promoting domains, and/or IgG Constant Domains could be utilized to generate alternative ROR1×CD3 bispecific three chain diabodies. For example, different anti-ROR1-VL and/or VH Domains were utilized to generate DART-A to DART-D (see, e.g., Table 9). Furthermore, any of the optimized anti-ROR1-VL and/or VH Domains provided herein may be used in place of anti-ROR1-VL(14) and anti-ROR1-VH(8) to generate alternative molecules.

C. ROR1×CD3×CD8 Trivalent Binding Molecules

Exemplary trivalent “ROR1×CD3×CD8” binding molecules having one binding site specific for ROR1 (comprising parental and/or optimized anti-ROR1-VL and anti-ROR1-VH Domains), one binding site specific for CD3 (comprising the VL and VH Domains of CD3 mAb 1 (D65G)), and one binding site specific for CD8 (comprising the VL and VH Domains of TRX₂) are provided. The exemplary trivalent binding molecules are designated as follows: “TRIDENT-A,” having three polypeptide chains and comprising the parental anti-ROR1-VL and anti-ROR1-VH Domains; “TRIDENT-B,” having four polypeptide chains and comprising the parental anti-ROR1-VL and anti-ROR1-VH Domains; “TRIDENT-C,” having three polypeptide chains and comprising the optimized anti-ROR1-VL(14) and anti-ROR1-VH(8) Domains; and “TRIDENT-D,” having four polypeptide chains and comprising the optimized anti-ROR1-VL(14) and anti-ROR1-VH(8) Domains. TRIDENT-A and TRIDENT-C have the general structure shown in FIG. 6D, and TRIDENT-B and TRIDENT-D have the general structure shown in FIG. 6A. The structure of these ROR1×CD3×CD8 trivalent binding molecules is detailed below. These exemplary ROR1×CD3×CD8 trivalent binding molecules are intended to illustrate, but in no way limit, the scope of the invention.

The first polypeptide chain of the exemplary ROR1×CD3×CD8 trivalent binding molecules having three or four polypeptide chains (see, e.g., FIG. 6A) comprises, in the N-terminal to C-terminal direction: an N-terminus; an anti-ROR1-VL Domain (SEQ ID NO:6 for TRIDENT-A; SEQ ID NO:6 for TRIDENT-B; SEQ ID NO:23 for TRIDENT-C; and SEQ ID NO:23 for TRIDENT-D); an intervening spacer peptide (Linker 1: GGGSGGGG (SEQ ID NO:33)); the VH Domain of CD3 mAb 1 (D65G) (SEQ ID NO:77); an intervening spacer peptide (Linker 2: ASTKG (SEQ ID NO:38)); a cysteine-containing Heterodimer-Promoting (E-coil) Domain (EVAACEK-EVAALEK-EVAALEK-EVAALEK (SEQ ID NO:48)); an intervening spacer peptide (Linker 3: GGGDKTHTCPPCP (SEQ ID NO:58)); a knob-bearing IgG1 CH2-CH3 Domain (SEQ ID NO:71); and a C-terminus. Encoding polynucleotides for this polypeptide chain may encode the C-terminal lysine residue of SEQ ID NO:71 (i.e., X of SEQ ID NO:71), however, as discussed above, this lysine residue may be post-translationally removed in some expression systems. Accordingly, the invention encompasses such a first polypeptide chain that contains such lysine residue (i.e., SEQ ID NO:71, wherein X is lysine), as well as a first polypeptide chain that lacks such lysine residue (i.e., SEQ ID NO:71, wherein X is absent). The anti-ROR1-VL Domain present in each trivalent binding molecule is indicated in Table 10 and the amino acid sequences are provided above.

The second polypeptide chain of the exemplary ROR1×CD3×CD8 trivalent binding molecules having three or four polypeptide chains comprises, in the N-terminal to C-terminal direction: an N-terminus; the VL Domain of CD3 mAb 1 (SEQ ID NO:75); an intervening spacer peptide (Linker 1: GGGSGGGG (SEQ ID NO:33)); an anti-ROR1-VH Domain (SEQ ID NO:7 for TRIDENT-A; SEQ ID NO:7 for TRIDENT-B; SEQ ID NO:31 for TRIDENT-C; and SEQ ID NO:31 for TRIDENT-D); an intervening spacer peptide (Linker 2: ASTKG (SEQ ID NO:38)); a cysteine-containing Heterodimer-Promoting (K-coil) Domain (KVAACKE-KVAALKE-KVAALKE-KVAALKE (SEQ ID NO:49)); and a C-terminus. The anti-ROR1-VH Domain present in each diabody is indicated in Table 10 and the amino acid sequences are provided above.

The third polypeptide chain of the exemplary three polypeptide chain ROR1×CD3×CD8 trivalent binding molecules TRIDENT-A and TRIDENT-C comprises, in the N-terminal to C-terminal direction: an N-terminus; the VL Domain of TRX₂ (SEQ ID NO:84); a intervening spacer peptide (Linker 4: GGGGSGGGGSGGGGS (SEQ ID NO:64)); the VH Domain of TRX₂ (SEQ ID NO:85); an intervening spacer peptide (Linker 3: VEPKSADKTHTCPPCP (SEQ ID NO:55); a hole-bearing IgG1 CH2-CH3 Domain (SEQ ID NO:72); and a C-terminus. Encoding polynucleotides for this polypeptide chain may encode the C-terminal lysine residue of SEQ ID NO:72 (i.e., X of SEQ ID NO:72), however, as discussed above, this lysine residue may be post-translationally removed in some expression systems. Accordingly, the invention encompasses such a third polypeptide chain that contains such lysine residue (i.e., SEQ ID NO:72, wherein X is lysine), as well as a third polypeptide chain that lacks such lysine residue (i.e., SEQ ID NO:72, wherein X is absent).

The third polypeptide chain of the exemplary four polypeptide chain ROR1×CD3×CD8 trivalent binding molecules TRIDENT-B and TRIDENT-D is an antibody heavy chain and comprises, in the N-terminal to C-terminal direction: an N-terminus, the VH Domain of TRX₂ (SEQ ID NO:85); an IgG1 CH1 Domain (SEQ ID NO:67); an IgG1 Hinge Region (EPKSCDKTHTCPPCP (SEQ ID NO:60)); a hole-bearing IgG1 CH2-CH3 Domain (SEQ ID NO:72); and a C-terminus. Encoding polynucleotides for this polypeptide chain may encode the C-terminal lysine residue of SEQ ID NO:72 (i.e., X of SEQ ID NO:72), however, as discussed above, this lysine residue may be post-translationally removed in some expression systems. Accordingly, the invention encompasses such a third polypeptide chain that contains such lysine residue (i.e., SEQ ID NO:72, wherein X is lysine), as well as a third polypeptide chain that lacks such lysine residue (i.e., SEQ ID NO:72, wherein X is absent).

The fourth polypeptide chain of the exemplary ROR1×CD3×CD8 trivalent binding molecules having four polypeptide chains (i.e., TRIDENT-B and TRIDENT-D) is an antibody light chain and comprises, in the N-terminal to C-terminal direction: an N-terminus, the VL Domain of CD8 mAb TRX₂ (SEQ ID NO:84); a CL Kappa Domain (SEQ ID NO:65); and a C-terminus.

The amino acid sequence of a representative ROR1×CD3×CD8 trivalent binding molecule having three polypeptide chains, TRIDENT-C, is provided. TRIDENT-C comprises the optimized anti-ROR1-VL and anti-ROR1-VH Domains anti-ROR1-VL(14) and anti-ROR1-VH(8), respectively.

### TRIDENT-A

The amino acid sequence of the first polypeptide chain of TRIDENT-A (SEQ ID NO:105) is shown below (parental anti-ROR1-VL is underlined):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLGGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of TRIDENT-A (SEQ ID NO:106) is shown below (parental anti-ROR1-VH is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWVRQAPG
KGLEWVATIY PSSGKTYYAD SVKGRFTISS DNAKNSLYLQ
MNSLRAEDTA VYYCARDSYA DDAALFDIWG QGTTVTVSSA
STKGKVAACK EKVAALKEKV AALKEKVAAL KE

The amino acid sequence of the third polypeptide chain of TRIDENT-A (SEQ ID NO:107) is shown below:

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGCG TKVEIKGGGG SGGGGSGGGG
SQVQLVESGG GVVQPGRSLR LSCAASGFTF SDFGMNWVRQ
APGKCLEWVA LIYYDGSNKF YADSVKGRFT ISRDNSKNTL
YLQMNSLRAE DTAVYYCAKP HYDGYYHFFD SWGQGTLVTV
SSVEPKSADK THTCPPCPAP EAAGGPSVFL FPPKPKDTLM
ISRTPEVTCV VVDVSHEDPE VKFNWYVDGV EVHNAKTKPR
EEQYNSTYRV VSVLTVLHQD WLNGKEYKCK VSNKALPAPI
EKTISKAKGQ PREPQVYTLP PSREEMTKNQ VSLSCAVKGF
YPSDIAVEWE SNGQPENNYK TTPPVLDSDG SFFLVSKLTV
DKSRWQQGNV FSCSVMHEAL HNRYTQKSLS LSPGK

### TRIDENT-B

The amino acid sequence of the first polypeptide chain of TRIDENT-B is the same as that of the first polypeptide chain of TRIDENT-A (SEQ ID NO:105). The amino acid sequence of the second polypeptide chain of TRIDENT-B is the same as that of the second polypeptide chain of TRIDENT-A (SEQ ID NO:106).

The amino acid sequence of the third polypeptide chain of TRIDENT-B (SEQ ID NO:108) is shown below:

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

The amino acid sequence of the fourth polypeptide chain of TRIDENT-B (SEQ ID NO:109) is shown below:

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

### TRIDENT-C

The amino acid sequence of the first polypeptide chain of TRIDENT-C (SEQ ID NO:110) is shown below (anti-ROR1-VL(14) is underlined):

QLVLTQSPSA SASLGSSVKL TCTLSSGHKT DTIDWYQQQP
GKAPRYLMKL EGSGSYNKGS GVPDRFSGSS SGADWYLTIS
SLQSEDEADY YCGTDYPGNY LFGGGTQLTV LGGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSASTKG EVAACEKEVA ALEKEVAALE KEVAALEKGG
GDKTHTCPPC PAPEAAGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSREEMT KNQVSLWCLV KGFYPSDIAV
EWESNGQPEN NYKTTPPVLD SDGSFFLYSK LTVDKSRWQQ
GNVFSCSVMH EALHNHYTQK SLSLSPGK

The amino acid sequence of the second polypeptide chain of TRIDENT-C (SEQ ID NO:111) is shown below (anti-ROR1-VH(8) is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWIRQAPG
KGLEWVATIY PSSGKTYYAD SAKGRLTISS DNAKDSLYLQ
MNSLRAEDTA VYYCTRDSYA DDAALFDIWG QGTTVTVSSA
STKGKVAACK EKVAALKEKV AALKEKVAAL KE

The amino acid sequence of the third polypeptide chain of TRIDENT-C is identical to that of the third polypeptide chain of TRIDENT-A (SEQ ID NO:107), provided above.

### TRIDENT-D

The first and second polypeptide chains of TRIDENT-D are identical to the first and second polypeptide chains of TRIDENT-C. Accordingly, the amino acid sequence of the first polypeptide chain of TRIDENT-D is SEQ ID NO:110, and the amino acid sequence of the second polypeptide chain of TRIDENT-D is SEQ ID NO:111, as provided above. The third and fourth polypeptide chains of TRIDENT-D are identical to the third and four polypeptide chains of TRIDENT-B (SEQ ID NOs:104 and 105, respectively), provided above.

It will be appreciated in view of the teachings provided herein that different domain orientations, VH Domains, VL Domains, linkers, and/or heterodimer promoting domains, could be utilized to generate alternative ROR1×CD3×CD8 trivalent binding molecules. For example, different anti-ROR1-VL and/or VH Domains were utilized to generate TRIDENT-A and TRIDENT-C (see, e.g., Table 10). Furthermore, any of the optimized anti-ROR1-VL and/or VH Domains provided herein may be used in place of anti-ROR1-VL(14) and anti-ROR1-VH(8) to generate alternative molecules.

### X. Methods of Production

The ROR1-binding molecules of the present invention are most preferably produced through the recombinant expression of nucleic acid molecules that encode such polypeptides, as is well-known in the art.

Polypeptides of the invention may be conveniently prepared using solid phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants {e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., humanized, single-chain, etc. are known in the art, and have been described above. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

Vectors containing polynucleotides of interest (e.g., polynucleotides encoding the polypeptide chains of the ROR1-binding molecules of the present invention) can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cell capable of overexpressing heterologous DNAs can be used for the purpose of expressing a polypeptide or protein of interest. Non-limiting examples of suitable mammalian host cells include but are not limited to COS, HeLa, and CHO cells.

The invention includes polypeptides comprising an amino acid sequence of an ROR1-binding molecule of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available.

The invention includes variants of ROR1-binding molecules, including functionally equivalent polypeptides that do not significantly affect the properties of such molecules as well as variants that have enhanced or decreased activity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs. Amino acid residues that can be conservatively substituted for one another include but are not limited to: glycine/alanine; serine/threonine; valine/isoleucine/leucine; asparagine/glutamine; aspartic acid/glutamic acid; lysine/arginine; and phenylalanine/tyrosine. These polypeptides also include glycosylated and non-glycosylated polypeptides, as well as polypeptides with other post-translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention encompasses fusion proteins comprising one or more of the optimized anti-ROR1-VL and/or VH of this invention. In one embodiment, a fusion polypeptide is provided that comprises a light chain, a heavy chain or both a light and heavy chain. In another embodiment, the fusion polypeptide contains a heterologous immunoglobulin constant region. In another embodiment, the fusion polypeptide contains a Light Chain Variable Domain and a Heavy Chain Variable Domain of an antibody produced from a publicly-deposited hybridoma. For purposes of this invention, an antibody fusion protein contains one or more polypeptide domains that specifically bind to ROR1 and another amino acid sequence to which it is not attached in the native molecule, for example, a heterologous sequence or a homologous sequence from another region.

The present invention particularly encompasses ROR1-binding molecules (e.g., antibodies, diabodies, trivalent binding molecules, etc.,) conjugated to a diagnostic or therapeutic moiety. For diagnostic purposes ROR1-binding molecules of the invention may be coupled to a detectable substance. Such ROR1-binding molecules are useful for monitoring and/or prognosing the development or progression of a disease as part of a clinical testing procedure, such as determining the efficacy of a particular therapy. Examples of detectable substances include various enzymes (e.g., horseradish peroxidase, beta-galactosidase, etc.), prosthetic groups (e.g., avidin/biotin), fluorescent materials (e.g., umbelliferone, fluorescein, or phycoerythrin), luminescent materials (e.g., luminol), bioluminescent materials (e.g., luciferase or aequorin), radioactive materials (e.g., carbon-14, manganese-54, strontium-85 or zinc-65), positron emitting metals, and nonradioactive paramagnetic metal ions. The detectable substance may be coupled or conjugated either directly to the ROR1-binding molecule or indirectly, through an intermediate (e.g., a linker) using techniques known in the art.

For therapeutic purposes ROR1-binding molecules of the invention may be conjugated to a therapeutic moiety such as a cytotoxin, (e.g., a cytostatic or cytocidal agent), a therapeutic agent or a radioactive metal ion, e.g., alpha-emitters. A cytotoxin or cytotoxic agent includes any agent that is detrimental to cells such as, for example, Pseudomonas exotoxin, Diptheria toxin, a botulinum toxin A through F, ricin abrin, saporin, and cytotoxic fragments of such agents. A therapeutic agent includes any agent having a therapeutic effect to prophylactically or therapeutically treat a disorder. Such therapeutic agents may be may be chemical therapeutic agents, protein or polypeptide therapeutic agents, and include therapeutic agents that possess a desired biological activity and/or modify a given biological response. Examples of therapeutic agents include alkylating agents, angiogenesis inhibitors, anti-mitotic agents, hormone therapy agents, and antibodies useful for the treatment of cell proliferative disorders. The therapeutic moiety may be coupled or conjugated either directly to the ROR1-binding molecule or indirectly, through an intermediate (e.g., a linker) using techniques known in the art.

### XI. Uses of the ROR1-Binding Molecules of the Present Invention

The present invention encompasses compositions, including pharmaceutical compositions, comprising the ROR1-binding molecules of the present invention (e.g., antibodies, bispecific antibodies, bispecific diabodies, trivalent binding molecules, etc.), polypeptides derived from such molecules, polynucleotides comprising sequences encoding such molecules or polypeptides, and other agents as described herein.

As provided herein, the ROR1-binding molecules of the present invention, comprising the optimized anti-ROR1-VL and/or VH Domains provided herein, have the ability to bind ROR1 present on the surface of a cell and induce antibody-dependent cell-mediated cytotoxicity (ADCC) and/or complement dependent cytotoxicity (CDC) and/or mediate redirected cell killing (e.g., redirected T-cell cytotoxicity).

Thus, ROR1-binding molecules of the present invention, comprising the optimized anti-ROR1-VL and/or VH Domains provided herein, have the ability to treat any disease or condition associated with or characterized by the expression of ROR1. As discussed above, ROR1 is an onco-embryonic antigen expressed in numerous blood and solid malignancies, that is associated with high-grade tumors exhibiting a less-differentiated morphology, and is correlated with poor clinical outcomes (see, e.g., Zhang, S., et al. (2012) “The Onco-Embryonic Antigen ROR1 Is Expressed by a Variety of Human Cancers,” Am J. Pathol. 6:1903-1910; Zhang, H. et al. (2014) “ROR1 Expression Correlated With Poor Clinical Outcome In Human Ovarian Cancer,” Sci Rep. 4:5811). Thus, without limitation, the ROR1-binding molecules of the present invention may be employed in the diagnosis or treatment of cancer, particularly a cancer characterized by the expression of ROR1.

The cancers that may be treated by the ROR1-binding molecules of the present invention include cancers characterized by the presence of a cancer cell selected from the group consisting of a cell of: an adrenal gland tumor, an AIDS-associated cancer, an alveolar soft part sarcoma, an astrocytic tumor, an adrenal cancer, a bladder cancer, a bone cancer, a brain and spinal cord cancer, a metastatic brain tumor, a B-cell cancer, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a hematological malignancy, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In particular, ROR1-binding molecules of the present invention may be used in the treatment of adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.

The bispecific ROR1-binding molecules of the present invention augment the cancer therapy provided by ROR1 by promoting the redirected killing of tumor cells that express the second specificity of such molecules (e.g., CD2, CD3, CD8, CD16, the T Cell Receptor (TCR), NKG2D, etc.). Such ROR1-binding molecules are particularly useful for the treatment of cancer.

In addition to their utility in therapy, the ROR1-binding molecules of the present invention may be detectably labeled and used in the diagnosis of cancer or in the imaging of tumors and tumor cells.

### XII. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the ROR1-binding molecules of the present invention, or a combination of such agents and a pharmaceutically acceptable carrier. Preferably, compositions of the invention comprise a prophylactically or therapeutically effective amount of the ROR1-binding molecules of the present invention and a pharmaceutically acceptable carrier. The invention also encompasses such pharmaceutical compositions that additionally include a second therapeutic antibody (e.g., tumor-specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund' s adjuvant (complete and incomplete), excipient, or vehicle with which the therapeutic is administered. Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with a ROR1-binding molecule of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the ROR1-binding molecules of the present invention. The kit can further comprise one or more other prophylactic and/or therapeutic agents useful for the treatment of cancer, in one or more containers.

### XIII. Methods of Administration

The compositions of the present invention may be provided for the treatment, prophylaxis, and amelioration of one or more symptoms associated with a disease, disorder or infection by administering to a subject an effective amount of a fusion protein or a conjugated molecule of the invention, or a pharmaceutical composition comprising a fusion protein or a conjugated molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the ROR1-binding molecules of the present invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that preparations of the ROR1-binding molecules of the present invention are packaged in a hermetically sealed container such as an ampoule or sachette indicating the quantity of the molecule. In one embodiment, such molecules are supplied as a dry sterilized lyophilized powder or water free concentrate in a hermetically sealed container and can be reconstituted, e.g., with water or saline to the appropriate concentration for administration to a subject. Preferably, the ROR1-binding molecules of the present invention are supplied as a dry sterile lyophilized powder in a hermetically sealed container.

The lyophilized preparations of the ROR1-binding molecules of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such ROR1-binding molecules when provided in liquid form are supplied in a hermetically sealed container.

The amount of such preparations of the invention that will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

As used herein, an “effective amount” of a pharmaceutical composition is an amount sufficient to effect beneficial or desired results including, without limitation, clinical results such as decreasing symptoms resulting from the disease, attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

An effective amount can be administered in one or more administrations. For purposes of this invention, an effective amount of drug, compound, or pharmaceutical composition is an amount sufficient to reduce the proliferation of (or the effect of) viral presence and to reduce and/or delay the development of the viral disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art.

For the ROR1-binding molecules encompassed by the invention, the dosage administered to a patient is preferably determined based upon the body weight (kg) of the recipient subject. For the ROR1-binding molecules encompassed by the invention, the dosage administered to a patient is typically from about 0.01 μg/kg to about 30 mg/kg or more of the subject's body weight.

The dosage and frequency of administration of a ROR1-binding molecule of the present invention may be reduced or altered by enhancing uptake and tissue penetration of the molecule by modifications such as, for example, lipidation.

The dosage of a ROR1-binding molecule of the invention administered to a patient may be calculated for use as a single agent therapy. Alternatively, the molecule may be used in combination with other therapeutic compositions and the dosage administered to a patient are lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, N.Y., pp. 353-365 (1989); Lopez-Berestein, ibid., pp. 3 17-327).

Where the composition of the invention is a nucleic acid encoding a ROR1-binding molecule of the present invention, the nucleic acid can be administered in vivo to promote expression of its encoded ROR1-binding molecule by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of a ROR1-binding molecule of the present invention can include a single treatment or, preferably, can include a series of treatments. In a preferred example, a subject is treated with such a diabody one time per week for between about 1 to 10 weeks, preferably between 2 to 8 weeks, more preferably between about 3 to 7 weeks, and even more preferably for about 4, 5, or 6 weeks. The pharmaceutical compositions of the invention can be administered once a day with such administration occurring once a week, twice a week, once every two weeks, once a month, once every six weeks, once every two months, twice a year or once per year, etc. Alternatively, the pharmaceutical compositions of the invention can be administered twice a day with such administration occurring once a week, twice a week, once every two weeks, once a month, once every six weeks, once every two months, twice a year or once per year, etc. Alternatively, the pharmaceutical compositions of the invention can be administered three times a day with such administration occurring once a week, twice a week, once every two weeks, once a month, once every six weeks, once every two months, twice a year or once per year, etc. It will also be appreciated that the effective dosage of the molecules used for treatment may increase or decrease over the course of a particular treatment.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### Optimization of Anti-ROR1-VL and Anti-ROR1-VH

In order to obtain optimized anti-ROR1 antibody species that exhibit improved affinity for human ROR1, polynucleotides encoding the parental anti-ROR1 antibody VL and anti-ROR1-VH Domains (i.e., anti-ROR1-VL or anti-ROR1-VH, respectively) were subjected to mutagenesis. The VL Domain variants were designated “anti-ROR1-VL(2),” “anti-ROR1-VL(3),” “anti-ROR1-VL(4),” “anti-ROR1-VL(5),” “anti-ROR1-VL(6),” “anti-ROR1-VL(7),” “anti-ROR1-VL(8),” “anti-ROR1-VL(9),” “anti-ROR1-VL(10),” “anti-ROR1-VL(11),” “anti-ROR1-VL(12),” “anti-ROR1-VL(13),” and “anti-ROR1-VL(14),”and the VH Domain variants were designated “anti-ROR1-VH(1),” “anti-ROR1-VH(2),” “anti-ROR1-VH(3),” “anti-ROR1-VH(4),” “anti-ROR1-VH(5),” “anti-ROR1-VH(6),” and “anti-ROR1-VH(7).” The amino acid sequences of these variants are provided above, the mutations and the corresponding SEQ ID NOs. are summarized in Table 6.

TABLE 6
Light Chain

| | Kabat Residue No: | | | | | | | | |
|---|---|---|---|---|---|---|---|---|---|
| | 17 | 20 | 49 | 54 | n/a | 66 | 71 | 92 | |
| | SEQ ID NO: 8 | | | | | | | | |
| | Residue No: | | | | | | | | SEQ |
| | 16 | 19 | 49 | 57 | 67 | 70 | 76 | 97 | ID |
| | (X₁) | (X₂) | (X₃) | (N) | (X₄) | (X₅) | (X₆) | (X₇) | NO: |
| Parental | S | K | K | N | G | S | R | Y | 6 |
| anti-ROR1-VL | | | | | | | | | |
| anti-ROR1-VH(1) | | | | | — | | | | 10 |
| anti-ROR1-VL(2) | | | | | | | W | | 11 |
| anti-ROR1-VL(3) | | | N | | | | | | 12 |
| anti-ROR1-VL(4) | G | | | | | | | | 13 |
| anti-ROR1-VL(5) | | | | S | | | | | 14 |
| anti-ROR1-VL(6) | | | | | | I | | | 15 |
| anti-ROR1-VL(7) | | I | | | | | | | 16 |
| anti-ROR1-VL(8) | | N | | | | | | | 17 |
| anti-ROR1-VL(9) | | | | T | | | | | 18 |
| anti-ROR1-VL(10) | | | | | | | | N | 19 |
| anti-ROR1-VL(11) | | | | | | | W | N | 20 |
| anti-ROR1-VL(12) | | | | | | I | W | | 21 |
| anti-ROR1-VL(13) | | | | | | I | W | N | 22 |
| anti-ROR1-VL(14) | | | | | — | | W | | 23 |

Heavy Chain

| | Kabat Residue No: | | | | | | |
|---|---|---|---|---|---|---|---|
| | 37 | 63 | 67 | 76 | 93 | 101 | |
| | SEQ ID NO: 9 | | | | | | |
| | Residue No. | | | | | | SEQ |
| | 37 | 64 | 68 | 77 | 97 | 109 | ID |
| | (X₁) | (X₂) | (X₃) | (X₄) | (X₅) | (D) | NO: |
| Parental | V | V | F | N | A | D | 7 |
| anti-ROR1-VH | | | | | | | |
| anti-ROR1-VH(1) | | | L | | | | 24 |
| anti-ROR1-VH(2) | | | | D | | | 25 |
| anti-ROR1-VH(3) | | | | | T | | 26 |
| anti-ROR1-VH(4) | | | | Y | | | 27 |
| anti-ROR1-VH(5) | | | | | | A | 28 |
| anti-ROR1-VH(6) | | | | | | Y | 29 |
| anti-ROR1-VH(7) | | | L | D | T | | 30 |
| anti-ROR1-VH(8) | I | A | L | D | T | | 31 |
| anti-ROR1-VH(9) | I | A | | | | | 32 |

Thirty-one ROR1×CD3 bispecific two chain covalently bonded diabodies were generated, each having one binding site specific for ROR1 comprising parental and/or variant anti-ROR1-VL and anti-ROR1-VH Domains, and one binding site specific for CD3 comprising the VL and VH Domains of CD3 mAb 1 (D65G). The general structure of the first and second polypeptide chains of these exemplary ROR1×CD3 bispecific two chain diabodies is provided in detail above. The particular anti-ROR1-VL and anti-ROR1-VH Domains present in each diabody (consecutively numbered and designated “DART-1” to “DART-31”) are provided in Table 7. The CD3 binding domain of such diabodies is the VL domain of CD3 mAb 1 (SEQ ID NO:75) or the VH Domain of anti-CD3 mAb 1 (D65G) (SEQ ID NO:77). The anti-ROR1 binding domain and anti-CD3 binding domain are separated from one another by an intervening spacer peptide (Linker 1) GGGSGGGG (SEQ ID NO:33).

TABLE 7

| DART | Anti- | Anti- | Mutation(s) | | kₐ | k_{d} | K_{D} |
|---|---|---|---|---|---|---|---|
| # | ROR1-VL | ROR1-VH | Kabat | ELISA | (×10⁵) | (×10⁻⁴) | (nM) |
| 1 | Parental | Parental | VL: parental | | 2.3 | 9.1 | 4 |
| | (SEQ ID | (SEQ ID | VH: parental | | | | |
| | NO: 6) | NO: 7) | | | | | |
| 2 | VL(2) | Parental | VL: R71W | ↑ | 3.2 | 4.7 | 1.5 |
| | (SEQ ID | (SEQ ID | VH: parental | | | | |
| | NO: 11) | NO: 7) | | | | | |
| 3 | VL(3) | Parental | VL: K49N | ✓ | 2.1 | 8.4 | 4 |
| | (SEQ ID | (SEQ ID | VH: parental | | | | |
| | NO: 12) | NO: 7) | | | | | |
| 4 | VL(4) | Parental | VL: S17G | ✓ | 2.3 | 8.5 | 3.7 |
| | (SEQ ID | (SEQ ID | VH: parental | | | | |
| | NO: 13) | NO: 7) | | | | | |
| 5 | VL(5) | Parental | VL: N54S | ✓ | 2.0 | 12 | 6.0 |
| | (SEQ ID | (SEQ ID | VH: parental | | | | |
| | NO: 14) | NO: 7) | | | | | |
| 6 | VL(6) | Parental | VL: S66I | ✓ | 2.1 | 12 | 5.7 |
| | (SEQ ID | (SEQ ID | VH: parental | | | | |
| | NO: 15) | NO: 7) | | | | | |
| 7 | VL(7) | Parental | VL: K20I | ✓ | — | — | — |
| | (SEQ ID | (SEQ ID | VH: parental | | | | |
| | NO: 16) | NO: 7) | | | | | |
| 8 | VL(8) | Parental | VL: K20N | ✓ | — | — | — |
| | (SEQ ID | (SEQ ID | VH: parental | | | | |
| | NO: 17) | NO: 7) | | | | | |
| 9 | VL(9) | Parental | VL: N54T | ✓ | — | — | — |
| | (SEQ ID | (SEQ ID | VH: parental | | | | |
| | NO: 18) | NO: 7) | | | | | |
| 10 | VL(10) | Parental | VL: Y92N | ✓ | 2.1 | 5.6 | 2.7 |
| | (SEQ ID | (SEQ ID | VH: parental | | | | |
| | NO: 19) | NO: 7) | | | | | |
| 11 | VL(11) | Parental | VL: R71W/Y92N | ↑ | 3.5 | 4.1 | 1.2 |
| | (SEQ ID | (SEQ ID | VH: parental | | | | |
| | NO: 20) | NO: 7) | | | | | |
| 12 | VL(12) | Parental | VL: S66I/R71W | ↑ | 3.5 | 5.8 | 1.7 |
| | (SEQ ID | (SEQ ID | VH: parental | | | | |
| | NO: 21) | NO: 7) | | | | | |
| 13 | VL(13) | Parental | VL: S66I/R71W/Y92N | ↑ | 3.5 | 4.9 | 1.4 |
| | (SEQ ID | (SEQ ID | VH: parental | | | | |
| | NO: 22) | NO: 7) | | | | | |
| 14 | Parental | VH(1) | VL: parental | ✓ | 2.2 | 9.8 | 4.5 |
| | (SEQ ID | (SEQ ID | VH: F67L | | | | |
| | NO: 6) | NO: 24) | | | | | |
| 15 | Parental | VH(2) | VL: parental | ✓ | 2.4 | 7.4 | 3.1 |
| | (SEQ ID | (SEQ ID | VH: N76D | | | | |
| | NO: 6) | NO: 25) | | | | | |
| 16 | Parental | VH(3) | VL: parental | ↑ | 2.3 | 9.9 | 4.3 |
| | (SEQ ID | (SEQ ID | VH: A93T | | | | |
| | NO: 6) | NO: 26) | | | | | |
| 17 | Parental | VH(4) | VL: parental | ✓ | 2.7 | 9.9 | 3.7 |
| | (SEQ ID | (SEQ ID | VH: N76Y | | | | |
| | NO: 6) | NO: 27) | | | | | |
| 18 | Parental | VH(5) | VL: parental | ↓ | — | — | — |
| | (SEQ ID | (SEQ ID | VH: D101A | | | | |
| | NO: 6) | NO: 28) | | | | | |
| 19 | Parental | VH(6) | VL: parental | ↓ | — | — | — |
| | (SEQ ID | (SEQ ID | VH: D101Y | | | | |
| | NO: 6) | NO: 29) | | | | | |
| 20 | VL(2) | VH(3) | VL: R71W | ↑ | 3.4 | 4.3 | 1.3 |
| | (SEQ ID | (SEQ ID | VH: A93T | | | | |
| | NO: 11) | NO: 26) | | | | | |
| 21 | VL(6) | VH(3) | VL: S66I | ↑ | 2.3 | 11 | 4.8 |
| | (SEQ ID | (SEQ ID | VH: A93T | | | | |
| | NO: 15) | NO: 26) | | | | | |
| 22 | VL(2) | VH(1) | VL: R71W | ↑ | 3.5 | 4.6 | 1.3 |
| | (SEQ ID | (SEQ ID | VH: F67L | | | | |
| | NO: 11) | NO: 24) | | | | | |
| 23 | VL(2) | VH(2) | VL: R71W | ↑ | 3.7 | 2.8 | 0.8 |
| | (SEQ ID | (SEQ ID | VH: N76D | | | | |
| | NO: 11) | NO: 25) | | | | | |
| 24 | VL(10) | VH(3) | VL: Y92N | ↑ | 2.3 | 5.4 | 2.4 |
| | (SEQ ID | (SEQ ID | VH: A93T | | | | |
| | NO: 19) | NO: 26) | | | | | |
| 25 | VL(2) | VH(7) | VL: R71W | ↑ | 3.8 | 4.5 | 1.2 |
| | (SEQ ID | (SEQ ID | VH: F67L/N76D/A93T | | | | |
| | NO: 11) | NO: 30) | | | | | |
| 26 | VL(11) | VH(3) | VL: R71W/Y92N | ↑ | 3.6 | 4.3 | 1.2 |
| | (SEQ ID | (SEQ ID | VH: A93T | | | | |
| | NO: 20) | NO: 26) | | | | | |
| 27 | VL(12) | VH(3) | VL: S66I R71W | ↑ | 3.8 | 4.5 | 1.2 |
| | (SEQ ID | (SEQ ID | VH: A93T | | | | |
| | NO: 21) | NO: 26) | | | | | |
| 28 | VL(13) | VH(3) | VL: S66I/R71W/Y92N | ↑ | 3.5 | 5.1 | 1.5 |
| | (SEQ ID | (SEQ ID | VH: A93T | | | | |
| | NO: 22) | NO: 26) | | | | | |
| 29 | VL(11) | VH(7) | VL: R71W/Y92N | ↑ | 3.8 | 4.8 | 1.3 |
| | (SEQ ID | (SEQ ID | VH: F67L/N76D/A93T | | | | |
| | NO: 20) | NO: 30) | | | | | |
| 30 | VL(12) | VH(7) | VL: S66I/R71W | ↑ | 3.8 | 4.7 | 1.2 |
| | (SEQ ID | (SEQ ID | VH: F67L N76D A93T | | | | |
| | NO: 21) | NO: 30) | | | | | |
| 31 | VL(13) | VH(7) | VL: S66I/R71W/Y92N | ↑ | 3.5 | 3.3 | 0.9 |
| | (SEQ ID | (SEQ ID | VH: F67L/N76D/A93T | | | | |
| | NO: 22) | NO: 30) | | | | | |
| 32 | VL(14) | VH(7) | VL:G deleted/R71W | — | 3.3 | 3.7 | 1.1 |
| | (SEQ ID | (SEQ ID | VH: F67L N76D A93T | | | | |
| | NO: 23) | NO: 30) | | | | | |
| 33 | VL(14) | VH(8) | VL:G deleted/R71W | — | 3.1 | 3.9 | 1.3 |
| | (SEQ ID | (SEQ ID | VH: V37I/V63A/ F67L | | | | |
| | NO: 23) | NO: 31) | N76D A93T | | | | |

✓: similar binding
↑: increased binding
↓: reduced binding
—: not determined

### DART-1

To illustrate, DART-1 comprises the parental anti-ROR1-VL and anti-ROR1-VL Domains. The amino acid sequence of DART-1 is provided below.

The amino acid sequence of the first polypeptide chain of DART-1 (SEQ ID NO:112) is shown below (the parental anti-ROR1-VL is shown in solid underline; the anti-CD3 binding domain is shown in dotted underline):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLGGGGSGGG

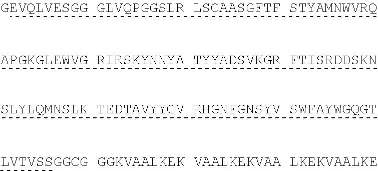

The amino acid sequence of the second polypeptide chain of DART-1 (SEQ ID NO:113) is shown below (the parental anti-ROR1-VH is shown in solid underline; the anti-CD3 binding domain is shown in dotted underline):

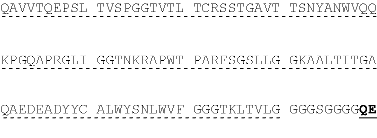

QLVESGGGLV QPGGSLRLSC AASGFTFSDY YMSWVRQAPG
KGLEWVATIY PSSGKTYYAD SVKGRFTISS DNAKNSLYLQ
MNSLRAEDTA VYYCARDSYL DDAALFDIWG QGTTVTVSSG
GCGGGEVAAL EKEVAALEKE VAALEKEVAA LEK

The amino acid sequences of the first and second polypeptide chains of a representative ROR1×CD3 bispecific two chain diabody comprising variant VL and VH Domains (i.e., anti-ROR1-VL(2) and anti-ROR1-VH(7)), DART-25, are provided above.

The binding of DART-1 to DART-31 to soluble human ROR1 was examined by ELISA. Briefly, microtiter plates were coated with a His-tagged soluble human ROR1 (“shROR1-His,” containing an extracellular portion of human ROR1 fused to a His-Tag) at 0.5 μg/mL, the plates were washed and incubated with three-fold serial dilutions of one of the generated diabodies (DART-1 to DART-31). The amount of diabody binding to the immobilized ROR1 was assessed using a biotinylated anti-E/K coil secondary antibody detected with Streptavidin-HRP. All samples were analyzed on a plate reader and binding curves generated. The binding of DART-2 to DART-31 relative to DART-1 is summarized in Table 7 above. A number of diabodies comprising variant VL and/or variant VH Domains exhibited improved binding relative to DART-1 indicating that such Variable Domains were optimized.

The binding kinetics of DART-1 to DART-6, DART-10 to DART-17, DART-20 to DART-33 was investigated using Biacore analysis in which ROR1 protein was passed over immobilized diabodies. Briefly, each diabody construct was captured on immobilized anti-E/K-coil surface and was incubated with shROR1-His at 25 and 100 nM, and the kinetics of binding were determined via Biacore analysis. The calculated kₐ, k_{d} and K_{D} from these studies are presented in Table 7. The majority of the mutations resulting in improved binding were located outside the CDRs. In particular, the single R71W substitution present in DART-2 (anti-ROR1-VL(2)) enhanced binding by more than two-fold in these studies.

In order to further characterize the variant anti-ROR1-VL and anti-ROR1-VH Domains, the ability of several ROR1×CD3 diabodies to mediate redirected cell killing was assessed using two different cytotoxic T lymphocyte (CTL) assays. In one assay ROR1×CD3 bispecific diabodies or a negative control diabody (lacking a ROR1-binding site) were incubated with effector pan T-cells and target tumor cells and the percentage cytotoxicity (i.e., cell killing) was determined by measuring the release of lactate dehydrogenase (LDH) into the media by damaged cells. These assays were performed using the CytoTox 96® Non-Radioactive Cytotoxicity Assay Kit (Promega) that quantitatively measures LDH release essentially as described below. Target cells (e.g., tumor target cells) at a density of 4×10⁵ cells/mL in assay media (RPMI 1640 without phenol red, 10% FBS, 1% pen/strep) and viability of higher than 90% at assay initiation, and isolated purified human T-cells suspended in the assay media at the appropriate density to achieve an effector-to-target (E:T) cell ratio of 10:1 (or the desired E:T ratio) are used. 50 μL target cell suspension (˜20,000 cells), 100 μL effector cell suspension (200,000 cells for 10:1 E:T ratio), and 50 μL serially diluted bispecific ROR1×CD3 diabody or a negative control diabody (lacking a ROR1-binding site) are added to duplicate wells of a microtiter plate and incubated (37° C. with 5% CO₂) for 24 hours. At the end of the incubation 30 μL lysis solution is added and the plates are incubated for 10 minutes to completely lyse the target cells. The plates are then centrifuged (212×g for 5 minutes) and 40 μL of supernatant is transferred from each well of the assay plate to a flat-bottom ELISA plate and 40 μL LDH substrate solution is added to each well. Plates are incubated for 10-20 minutes at room temperature in the dark and 40 μL of stop solution (Promega Cat #G183A) is added. The optical density is measured at 490 nm within 1 hour on a Victor2 Multilabel plate reader (Perkin Elmer #1420-014). Specific cell lysis is calculated from optical density (OD) data using the following formula:

$$Cytotoxicity ( \% ) = \frac{100 \times ( O D of Sample - O D of AICC )}{OF of MR - OD of SR}$$

and the dose-response curves are generated using GraphPad Prism 6 software by curve fitting the cytotoxicity values to the sigmoidal dose-response function.

In another assay, ROR1×CD3 bispecific diabodies, or a negative control diabody (lacking a ROR1 binding site), were incubated with pan T cells and target JIMT-1 cells that had been engineered to express the luciferase (luc) reporter gene (JIMT-1-Luc cells) and cytotoxicity was determined by luminescence (LUM) assay measuring cellular luciferase activity of the target cells. The preparation and set up for these assays is essentially identical to the LDH assay described above. Following incubation, 100 μL of culture medium is removed from each well and 100 μL Steady-Glo luciferase substrate is subsequently added to each well, followed by pipetting up/down several times for complete lysis of target cells. The plates are incubated at room temperature in the dark for 10 minutes and then luminescence intensity is measured using a VictorX₄ Multilabel plate reader (Perkin Elmer #1420-014) with luminescence relative light unit (RLU) as the read-out. RLU is indicative of relative viability of the target cells. Dose-response curves are generated using GraphPad Prism 6 software by curve fitting the RLU values to the sigmoidal dose-response function.

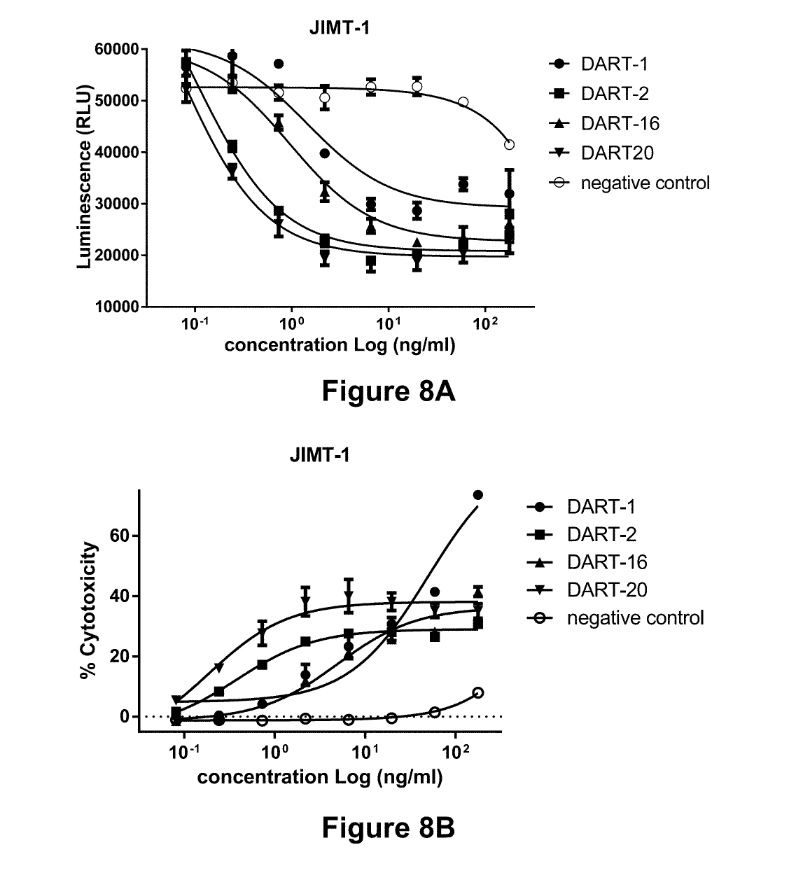

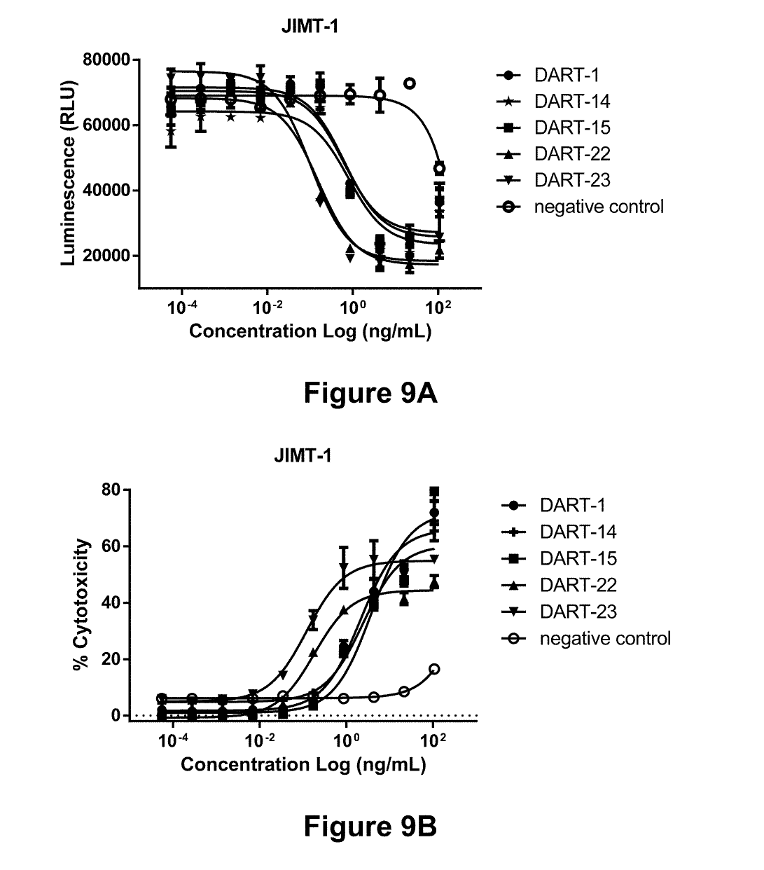

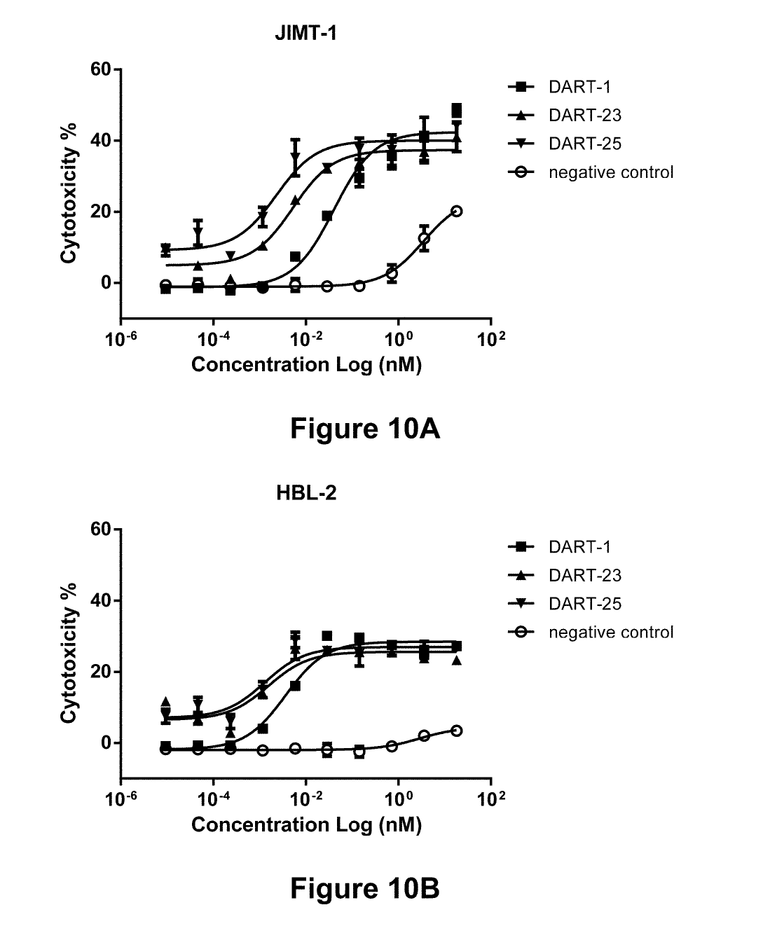

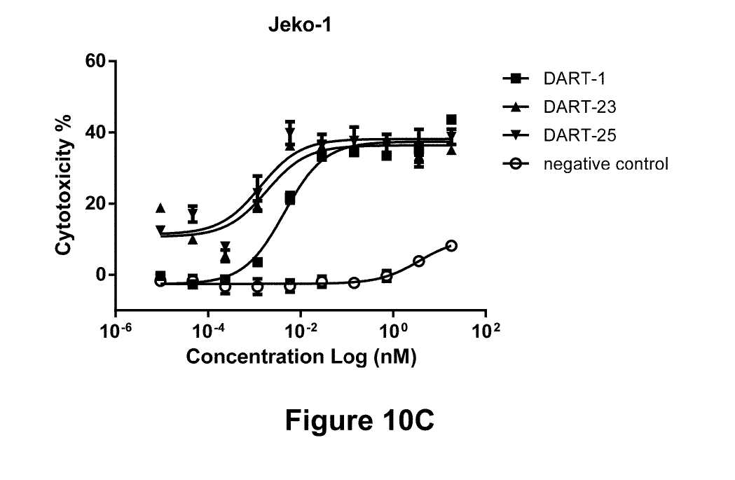

For these studies JIMT-1 breast carcinoma cells, HBL-2 mantle cell lymphoma cells or Jeko-1 mantle cell lymphoma cells were used as tumor target cells, and five-fold serial dilutions of the diabodies (DART-1, DART-2, DART-14, DART-15, DART-16, DART-20, DART-22, DART-23, and DART-25) were utilized. Representative cytotoxicity curves are presented in FIGS. 8A-8B, 9A-9B, and 10A-10C. The EC50 and maximum response values for the curves in FIGS. 10A-10C are provide in Table 8. These studies demonstrate that diabodies comprising optimized anti-ROR1-VL and/or VH Domains (e.g., DART-2, DART-8, DART-20, DART-22, DART-23, and DART-25) exhibit superior ability to mediate redirected cell killing of tumor cells relative to a diabody having the parental anti-ROR1-VL and/or VH Domains. In particular, diabodies having higher affinity for ROR1 than DART-1, and those comprising the A93T in the VH Domain exhibited enhanced ability to mediate redirected cell killing. DART-23, and DART-25 had EC50 values that were 10 to 20-fold lower than DART-1.

TABLE 8

| | DART-1 | | DART-23 | | DART-25 | |
|---|---|---|---|---|---|---|
| | Anti-ROR1-VL | | | | | |
| | Parental | | Anti-ROR1-VL(2) | | Anti-ROR1-VL(2) | |
| | (SEQ ID NO: 6) | | (SEQ ID NO: 11) | | (SEQ ID NO: 11) | |
| | Anti-ROR1-VH | | | | | |
| | Parental | | Anti-ROR1-VH(2) | | Anti-ROR1-VH(7) | |
| | (SEQ ID NO: 7) | | (SEQ ID NO: 25) | | (SEQ ID NO: 30) | |
| | EC50 | MR(%) | EC50 | MR(%) | EC50 | MR(%) |
| JIMT-1 | 0.0406 | 42.41 | 0.0053 | 37.33 | 0.0023 | 40.01 |
| HBL-2 | 0.0036 | 28.48 | 0.0016 | 25.61 | 0.0013 | 26.97 |
| Jeko-1 | 0.0044 | 37.4 | 0.0018 | 36.36 | 0.0014 | 38.17 |

### Example 2

### Further Optimization of Anti-ROR1 Variable Domains and Generation of Bispecific Three Chain Diabodies

To further optimize the anti-ROR1-VL and anti-ROR1-VH Domains, several changes were introduced into the anti-ROR1-Variable Domains to reduce immunogenicity. The parental anti-ROR1-VL Domain and the optimized anti-ROR1-VL(2) Domain were modified to remove an extra glycine (G) residue present between Kabat positions 63 and 64 (corresponding to position 67 of SEQ ID NO:6 and SEQ ID NO:11). The resulting anti-ROR1-VL Domains, designated “anti-ROR1-VL(1)” and “anti-ROR1-VL(14)” (SEQ ID NO:10 and SEQ ID NO:23, respectively, also see Table 6 above), were incorporated into ROR1×CD3 bispecific diabodies having two or three polypeptide chains and paired with different anti-ROR1-VH Domains as described in more detail below.

The anti-ROR1-VH Domain of such molecules was modified to remove two promiscuous high affinity MHC class II binding sequences present in CDR_{H}1 and CDR_{H}2. Specifically, the valine at Kabat position 37 (corresponding to position 37 of SEQ ID NO:7) was mutated to an isoleucine (“V371”) to disrupt the immunogenic sequence present in CDR_{H}1 and the valine at Kabat position 63 (corresponding to position 64 of SEQ ID NO:7) was mutated to alanine (“V63A”) to disrupt the immunogenic sequence present in CDR_{H}2. The resulting VH Domain designated “anti-ROR1-VH(8)” (SEQ ID NO:31, and see Table 6 above) was incorporated into ROR1×CD3 bispecific diabodies having two or three chains as described in more detail below.

Two ROR1×CD3 bispecific diabody having two chains were generated comprising anti-ROR1-VL(14). These diabodies were designated: “DART-32,” comprising anti-ROR1-VL(14) and anti-ROR1-VH(7); and “DART-33,” comprising anti-ROR1-VL(14) and anti-ROR1-VH(8) (see Table 7, above). The general structure of the first and second polypeptide chains of these exemplary ROR1×CD3 bispecific two chain diabodies is provided in detail above.

### DART-32

The amino acid sequence of the first polypeptide chain of DART-32 (SEQ ID NO:114) is shown below (anti-ROR1-VL(14) is underlined):

QLVLTQSPSA SASLGSSVKL TCTLSSGHKT DTIDWYQQQP
GKAPRYLMKL EGSGSYNKGS GVPDRFSGSS SGADWYLTIS
SLQSEDEADY YCGTDYPGNY LFGGGTQLTV LGGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSGGCGG GKVAALKEKV AALKEKVAAL KEKVAALKE

The amino acid sequence of the second polypeptide chain of DART-32 is identical to the second polypeptide chain of DART-25 (SEQ ID NO:97) provided above.

### DART-33

The amino acid sequence of the first polypeptide chain of DART-33 is identical to the first polypeptide of DART-32 (SEQ ID NO:114) provided above.

The amino acid sequence of the second polypeptide chain of DART-33 (SEQ ID NO:115) is shown below (anti-ROR1-VH(8) is underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWIRQAPG
KGLEWVATIY PSSGKTYYAD SAKGRLTISS DNAKDSLYLQ
MNSLRAEDTA VYYCTRDSYA DDAALFDIWG QGTTVTVSSG
GCGGGEVAAL EKEVAALEKE VAALEKEVAA LEK

In addition, four ROR1×CD3 bispecific diabodies having three chains and possessing an Fc Region were generated and designated: “DART-A,” comprising the parental anti-ROR1-VL (SEQ ID NO:6) and anti-ROR1-VH (SEQ ID NO:7) Domains; “DART-B,” comprising anti-ROR1-VL(1) (SEQ ID NO:10) and the parental anti-ROR1-VH (SEQ ID NO:7) Domain; “DART-C,” comprising anti-ROR1-VL(14) (SEQ ID NO:23) and anti-ROR1-VH(7) (SEQ ID NO:30); and “DART-D,” comprising anti-ROR1-VL(14) (SEQ ID NO:23) and anti-ROR1-VH(8) (SEQ ID NO:31). The general structure and amino acid sequences of the first, second and third polypeptide chains of these exemplary ROR1×CD3 bispecific three chain diabodies is provided in detail above. The particular anti-ROR1-VL and anti-ROR1-VH Domains present in DART-A, DART-B, DART-C, and DART-D are provided in Table 9.

TABLE 9

| DART | anti- | anti- | Mutation(s) | kₐ | k_{d} | K_{D} |
|---|---|---|---|---|---|---|
| # | ROR1-VL | ROR1-VH | Kabat | (×10⁵) | (×10⁻⁴) | (nM) |
| A | Parental | Parental | VL: parental | 10 | 6.9 | 0.69 |
| | (SEQ ID | (SEQ ID | VH: parental | | | |
| | NO: 6) | NO: 7) | | | | |
| B | VL(1) | Parental | VL: G deleted | 11 | 7.1 | 0.65 |
| | (SEQ ID | (SEQ ID | VH: parental | | | |
| | NO: 10) | NO: 7) | | | | |
| C | VL(14) | VH(7) | VL: G deleted/R71W | 17 | 4.3 | 0.25 |
| | (SEQ ID | (SEQ ID | VH: F67L N76D A93T | | | |
| | NO: 23) | NO: 30) | | | | |
| D | VL(14) | VH(8) | VL: G deleted/R71W | — | — | — |
| | (SEQ ID | (SEQ ID | VH: V37I/V63A/F67L | | | |
| | NO: 23) | NO: 31) | N76D A93T | | | |

—: not determined

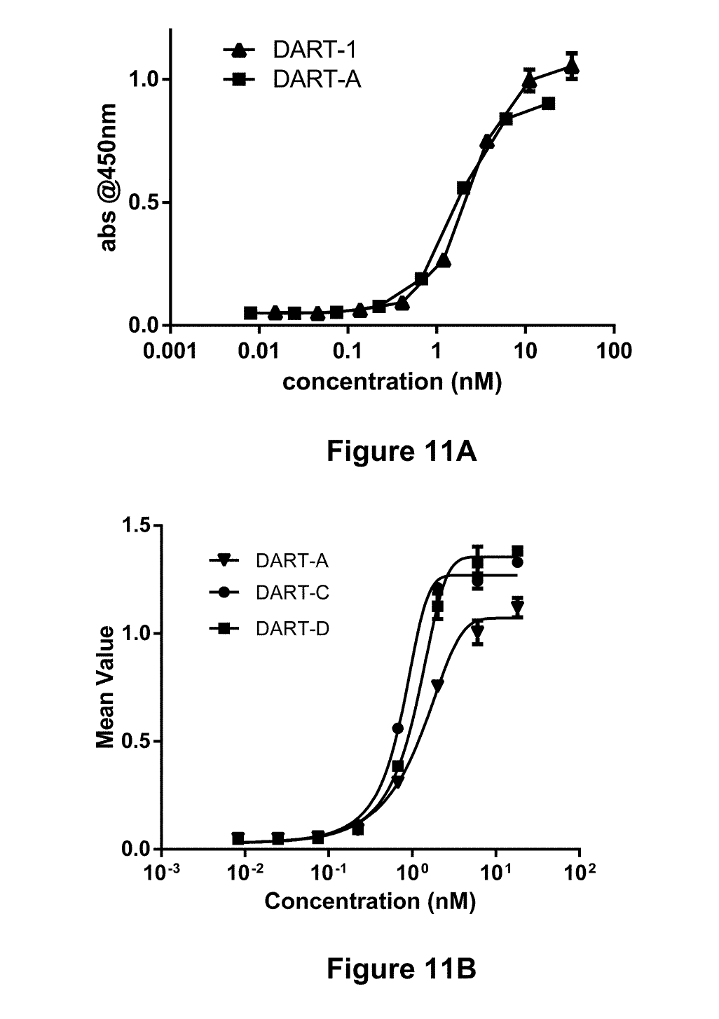

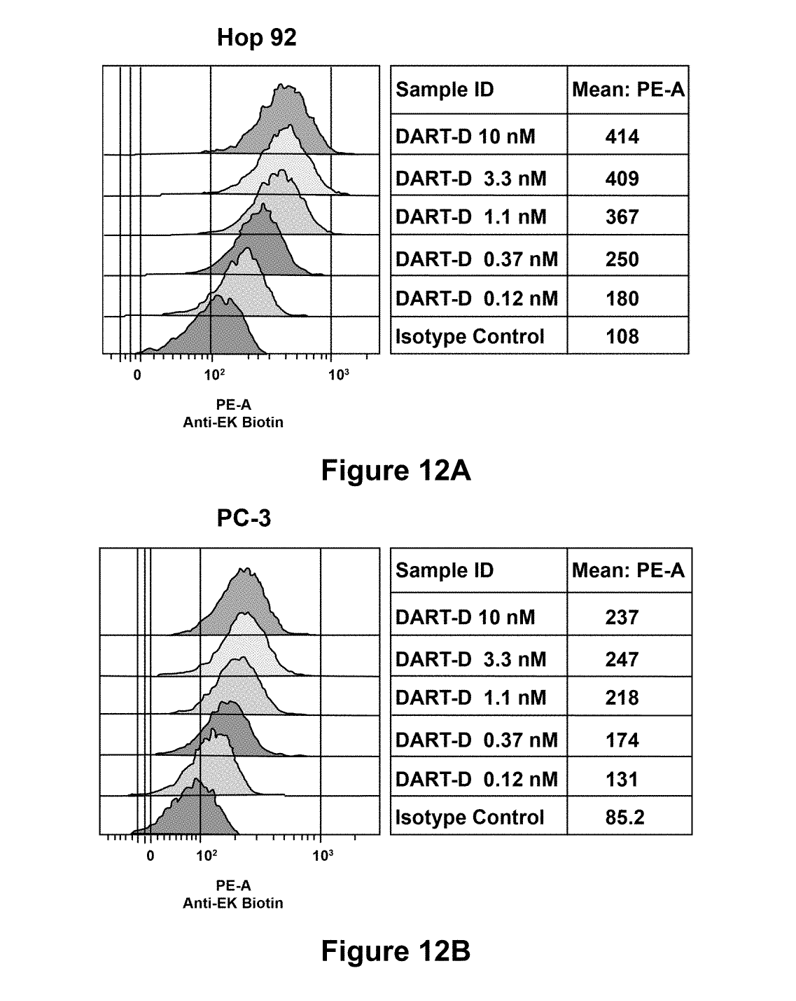

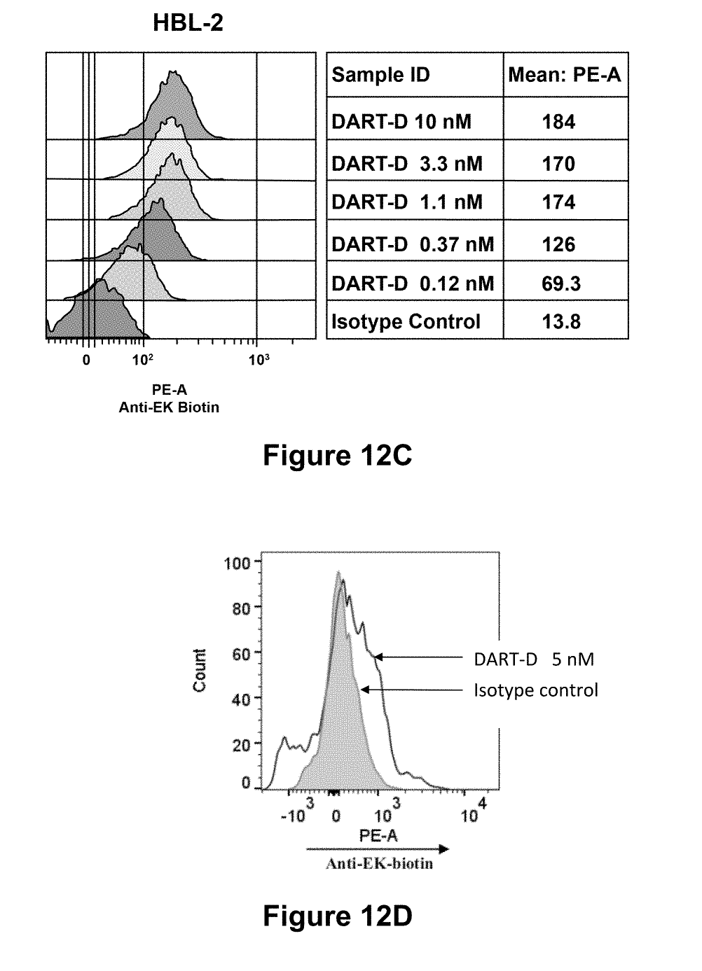

The ability of the bispecific ROR1×CD3 two and three chain diabodies DART-1 and DART-A to bind both ROR1 and CD3 was examined by a sandwich ELISA. Briefly, microtiter plates were coated with shROR1-His, the plates were washed and incubated with three-fold serial dilutions of DART-1 or DART-A. The amount of diabody binding to the immobilized ROR1 was assessed using a biotinylated CD3 detected with Streptavidin-HRP. All samples were analyzed on a plate reader and binding curves generated. In additional studies the ability of the bispecific ROR1×CD3 three chain diabodies DART-A, DART-C and DART-D to bind both ROR1 and CD3 was examined essentially as described above. The binding curves from these studies (FIG. 11A-11B) demonstrate that both two chain and three chain diabodies are capable of dual antigen binding and that dual antigen binding is retained in three chain diabodies having optimized anti-ROR1-VL and anti-ROR1-VH Domains. The ability of DART-D to bind to the surface of three ROR1-expressing cancer cell lines (HOP-92, PC-3 and HBL-2), and CD3-expressing human primary T cells was evaluated by FACS analysis. Briefly, cells (0.5 to 1.0×10⁶ cells/mL in 100 μL) were incubated with 0.12nM-10 nM DART-D (in FACS buffer containing 10% human AB serum, 100 μL final volume) in microtiter plates, for 20-60 minutes. The cells were washed twice incubated with biotin-conjugated mouse anti-EK-coil antibody that recognizes the E-coil/K-coil (EK) heterodimerization region (100 μL of 1 μg/mL mixed with 1:500 diluted streptavidin-phycoerythrin) for 45 min. The cells were then washed and resuspended with FACS buffer, and analyzed with a BD FCS Canto II flow cytometer using FlowJo v10 software. As demonstrated in FIGS. 12A-12D, DART-D bound to both human ROR1-expressing cancer cells (FIGS. 12A-12C) and to CD3-expressing T cells (FIG. 12D).

The binding kinetics of DART-32 and DART-33 was investigated using Biacore analysis in which shROR1-His was passed over immobilized diabodies as described in Example 1. The calculated kₐ, k_{d} and K_{D} from these studies are presented above in Table 7.

The binding affinity of DART-1, DART-A, DART-B, and DART-C was investigated using Biacore analysis in which each diabody construct was passed over immobilized ROR1. Briefly, shROR1-His was captured on immobilized anti-PentaHis surface and was incubated with DART-1, DART-A, DART-B or DART-C at 6.25-100 nM, and the kinetics of binding was determined via Biacore analysis. The calculated kₐ, k_{d} and K_{D} from these studies are presented in Table 8.

These studies demonstrate that the binding affinity of a three chain diabody such as DART-1, is comparable to that of a two chain diabody comprising the same VL and VH such as DART-A (see, Table 7). The binding affinities of DART-25 and DART-32 were nearly identical as were the binding affinities of DART-A and DART-B, demonstrating that deletion of the extra G residue does not alter the binding affinity (see, Table 7 and Table 8). Moreover, the binding of DART-32 and DART-C were enhanced by more than two-fold as compared to the corresponding diabodies comprising the parental VL and anti-ROR1-VH Domains (DART-1 and DART-A) (see, Table 7 and Table 8). In addition, DART-32 and DART-33 had nearly identical binding affinities (see, Table 7) demonstrating that the introduction of deimmunizing mutations adjacent to CDR_{H}1 and within CDR_{H}2 had no negative impact on the binding affinity.

### Example 3

### Cytotoxicity Studies

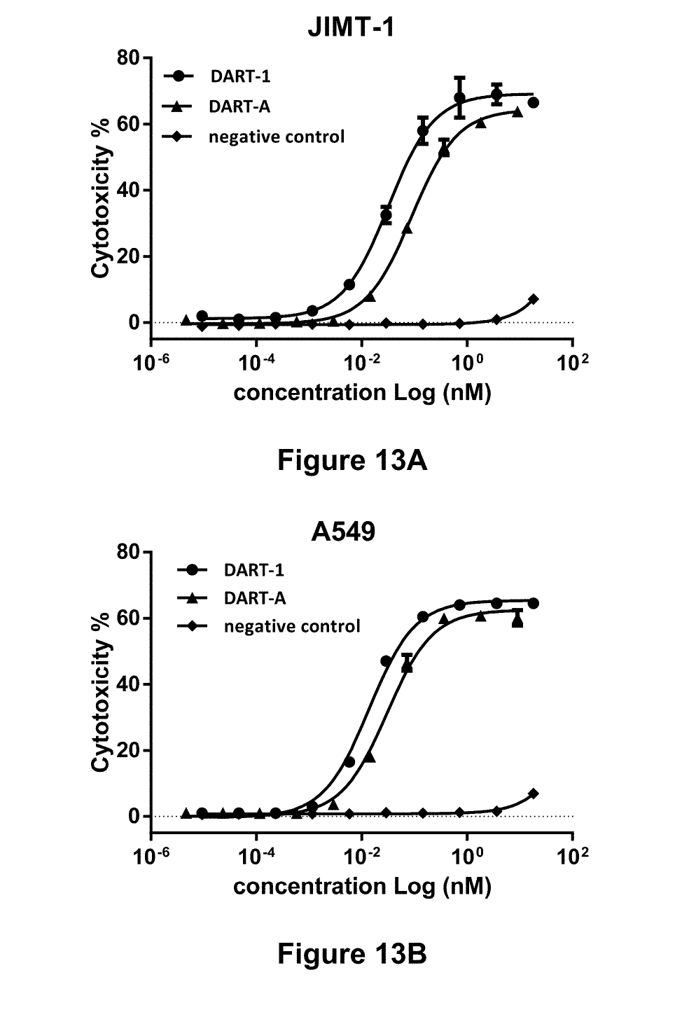

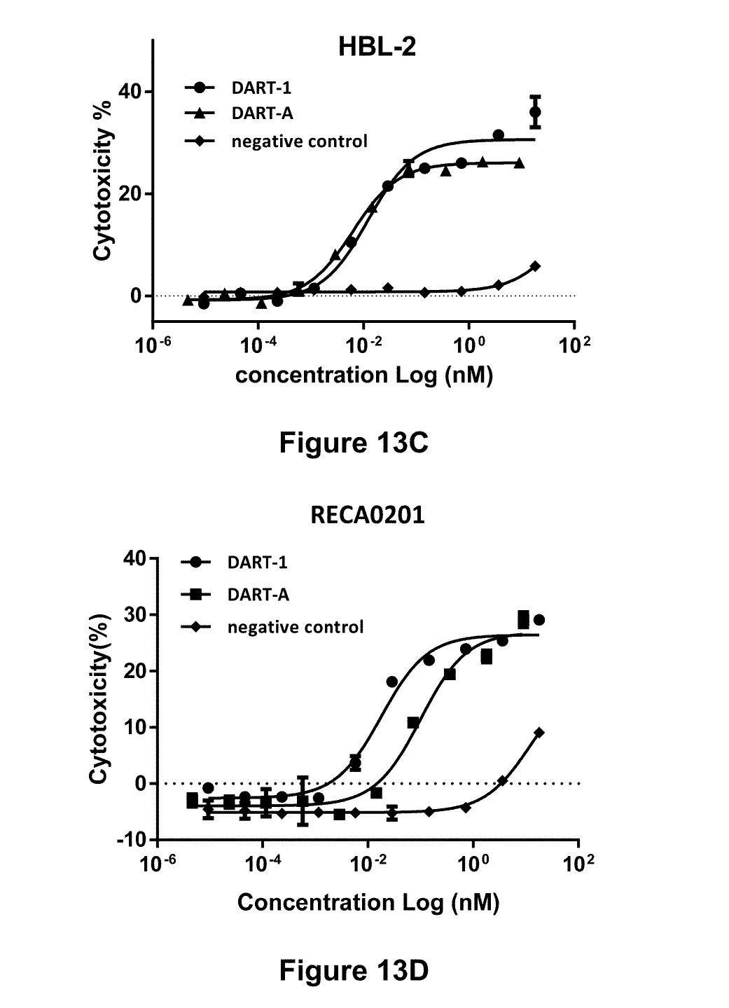

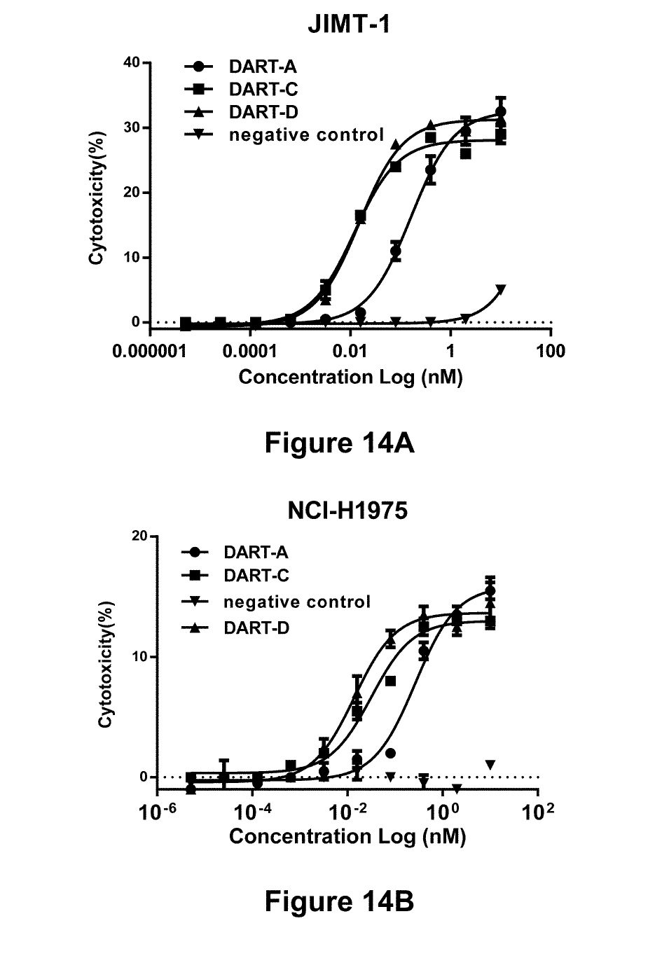

The ability of the bispecific ROR1×CD3 two and three chain diabodies DART-1 and DART-A to mediate redirected cell killing was assessed using the LDH release assay essentially as described in Example 1. For these studies ROR1×CD3 bispecific diabodies or a negative control diabody (lacking a ROR1-binding site) were incubated for 24 hours with effector pan T-cells and target tumor cells (JIMT-1 breast cancer cells, A549 lung cancer cells, HBL-2 mantle cell lymphoma cells) at an effector to target ratio of 10:1. In other studies, effector PBMC cells and target RECA0201 cancer stem cells were used at an effector to target ratio of 30:1. Five fold serial dilutions of DART-1, DART-A, and the negative control were utilized. Representative cytotoxicity curves for each target tumor cell type are presented in FIGS. 13A-13D. In further studies, the ability of the bispecific ROR1×CD3 three chain diabodies DART-A, DART-C and DART-D to mediate cytotoxicity was assessed using the LDH release assay described in Example 1. For these studies ROR1×CD3 bispecific diabodies or a negative control (lacking a ROR1-binding site) were incubated for 24 hours with effector pan T-cells and target tumor cells (JIMT-1 breast cancer cells, NCI-H1957 cells) at an effector to target ratio of 10:1. Five-fold serial dilutions of DART-A, DART-C, DART-D and the negative control were utilized. Representative cytotoxicity curves for each target tumor cell type are presented in FIGS. 14A-14B. No cell killing is observed in the absence of effector cells. These studies demonstrate that three chain diabodies retain the ability to mediate cytotoxicity, and that three chain diabodies having optimized anti-ROR1-VL and anti-ROR1-VH Domains retain the enhanced ability to mediate redirected cell killing seen in the two-chain format.

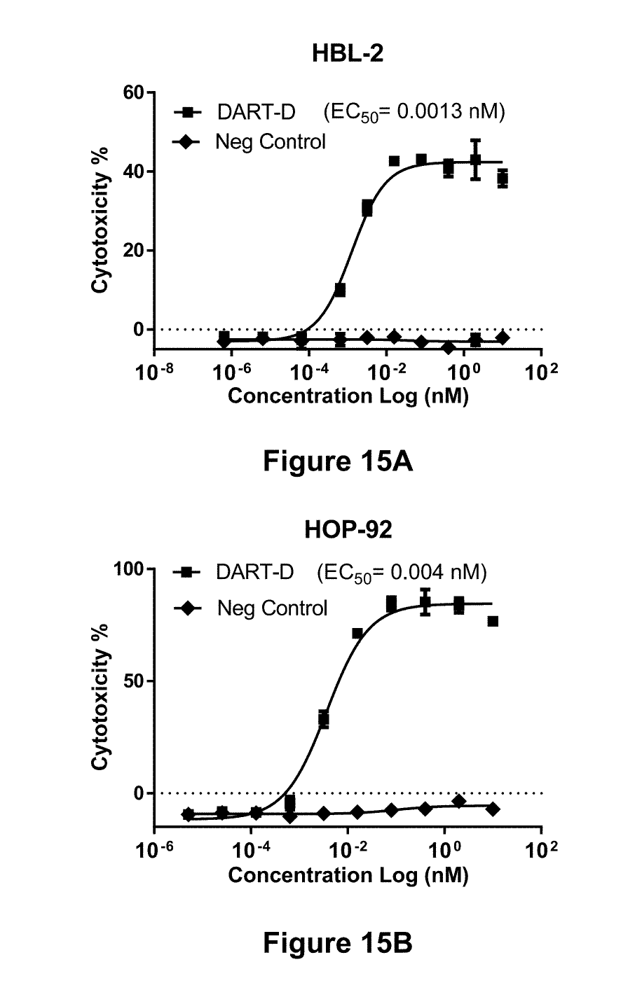

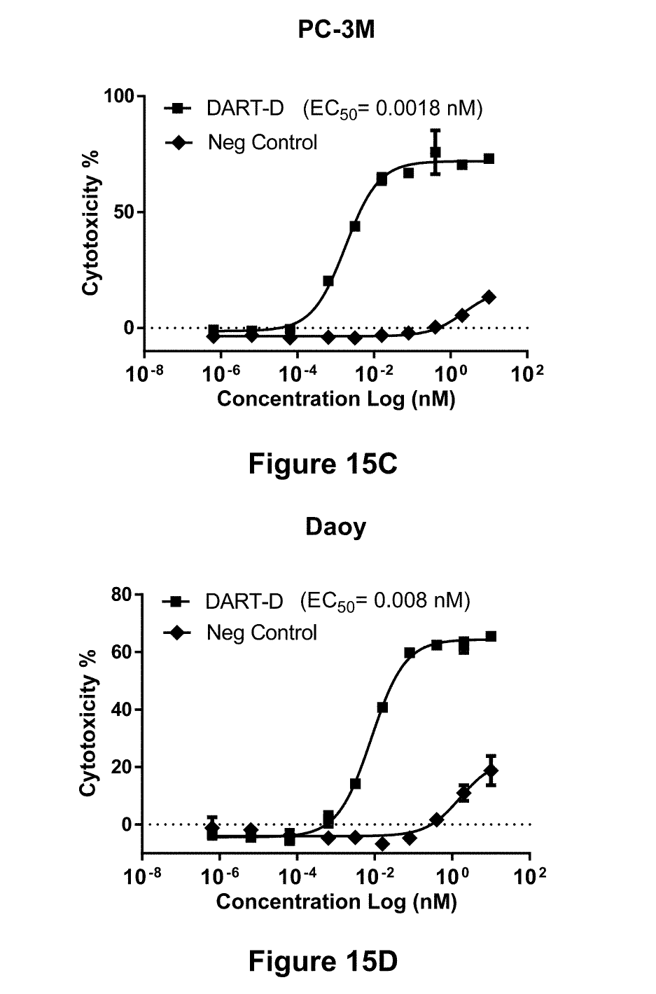

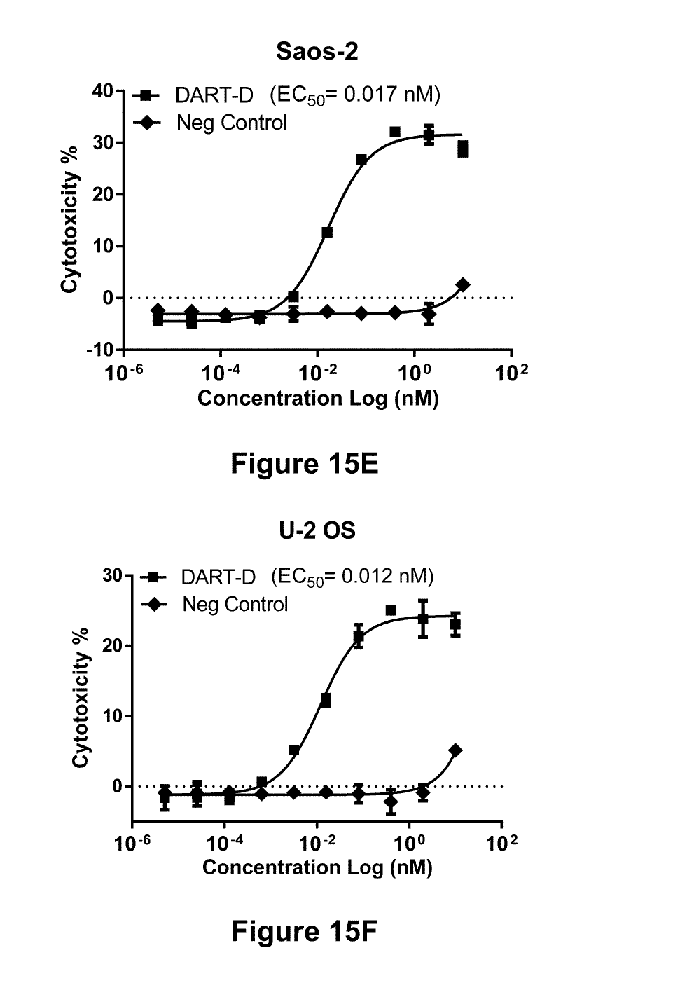

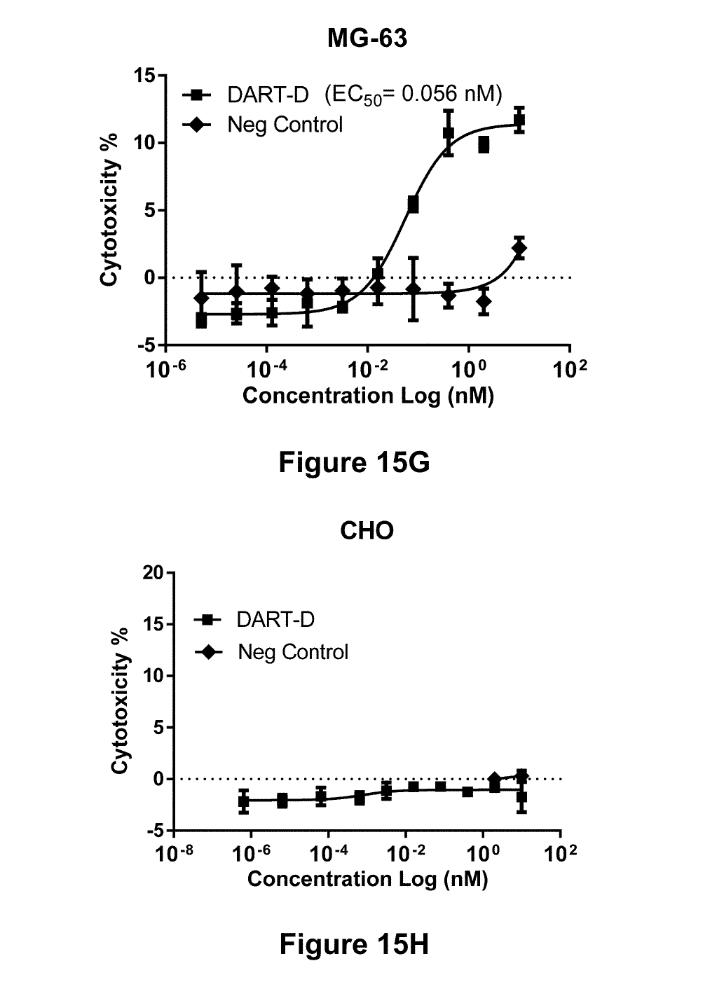

In additional studies, the cytotoxic activity of a representative bispecific ROR1×CD3 three chain diabody (DART-D; 5-fold serial dilutions) was evaluated using additional target tumor cell types: HBL-2 B-cell lymphoma cells; HOP-92 lung adenocarcinoma cells; PC-3M prostate cancer cells; Daoy medulloblastoma cells; and Saos-2, U-2 OS, and MG-63 bone osteosarcoma cells. CHO cells were also included in these studies as a ROR1 negative control target cells. For these studies, primary T cells from different donors were used in separate experiments. Primary T cells different donors were used sometimes for different target cell lines. The number of donors tested for each cell line as follows: MG-63 (2 donors), Saos-2 (5 donors), U2-OS (2 donors), HBL-2 (3 donors), HOP-92 (5 donors), Daoy (3 donors) and PC-3 (7 donors). Dose-dependent killing curves with T cells from a representative donor for each target cell type are shown FIGS. 15A-15H. EC₅₀ values (presented in parenthesis in each graph) ranging from 0.0013-0.056 nM were observed across the 7 target cell lines evaluated, with HBL-2 being the most sensitive cell line (EC₅₀=0.0013 ng/mL). At the highest concentration evaluated (10,000 ng/mL), minimal or no activity was observed with the control DART molecule. No cytotoxicity was observed in the presence of DART-D in ROR1-negative CHO cells confirming the specificity of the activity of the bispecific ROR1×CD3 diabodies to ROR1-expressing target cells. These studies further confirm that bispecific ROR1×CD3 three chain diabodies (e.g., DART-D) mediate potent, specific redirected killing of ROR1-expressing target cells.

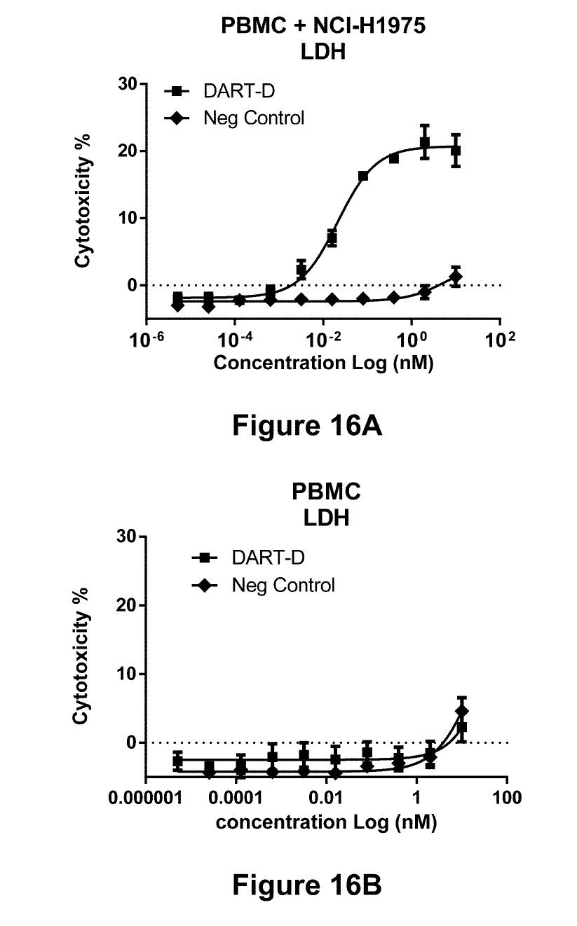

The level of T-cell activation induced by a representative bispecific ROR1×CD3 three chain diabody (DART-D) was evaluated in human PBMCs either alone or in the presence of ROR1-expressing target cells (NCI-H1975 lung cancer cells) at an E:T cell ratio of 10:1 by FACS. Briefly, PBMCs (200,000 cells/well in 100-150 μL of assay medium (RPMI 1640+10% FBS)) alone or with target cells (20,000 cells/well in 50 μL) were incubated with serial dilutions of DART-D at the indicated concentrations in duplicate wells of a microtiter plate for 24 hours at 37° C. 40 μL of supernatant from each well was used for LDH release measurement as detailed above, the remaining supernatant was used for measuring cytokines. Briefly, cells were labeled in the assay plate with CD8-FITC, CD4-APC, CD25-PE, and CD69-PECy5 antibodies (BD Biosciences) in FACS buffer (100 μL/well). The plates were incubated (in the dark at 4° C.) for 30 minutes. The cells were then washed and resuspended in FACS buffer and analyzed essentially as described in Example 2 above. In addition, IFN-γ, IL-2, IL-4, IL-6, IL-10, and TNF-α cytokine levels were measured in culture supernatants collected from same experiment using the BD CBA Human Th1/Th2 Cytokine Kit according to the manufacturer's instructions. Cytokine concentrations were determined using FCAP Array (v3.0.1, BD Biosciences). Values outside the range of concentrations of standards (0-5000 pg/mL) were extrapolated from a 4-parameter standard curve using sample intensity values. The results of these studies are presented in FIGS. 16A-16B, 17A-17D, and 18A-18E.

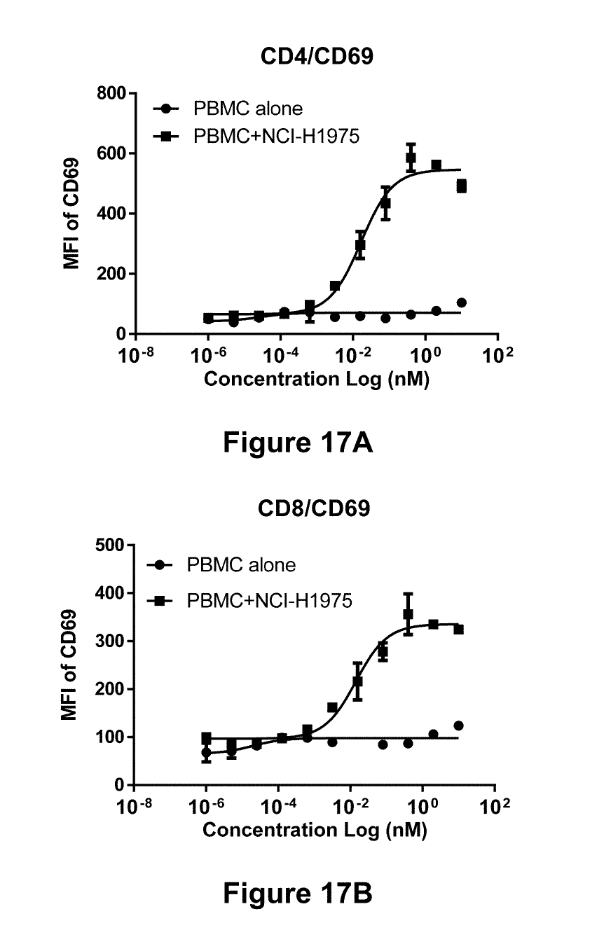

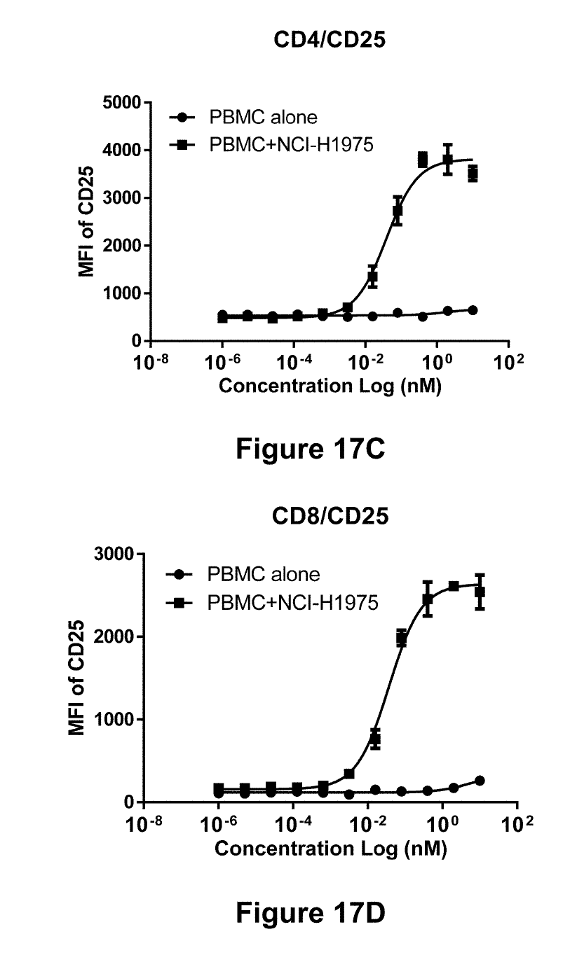

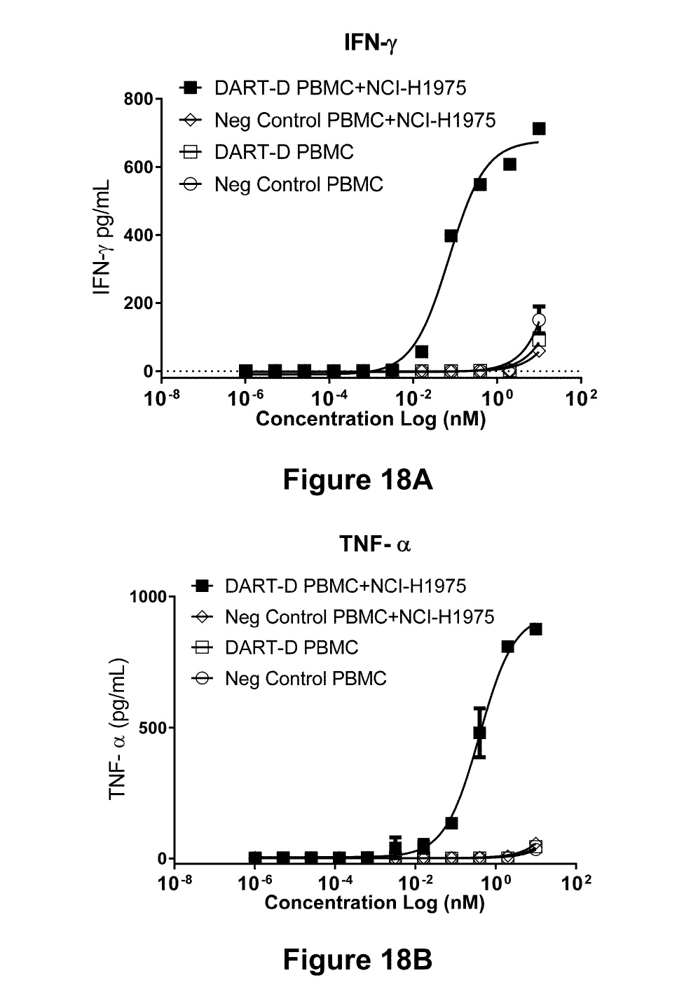

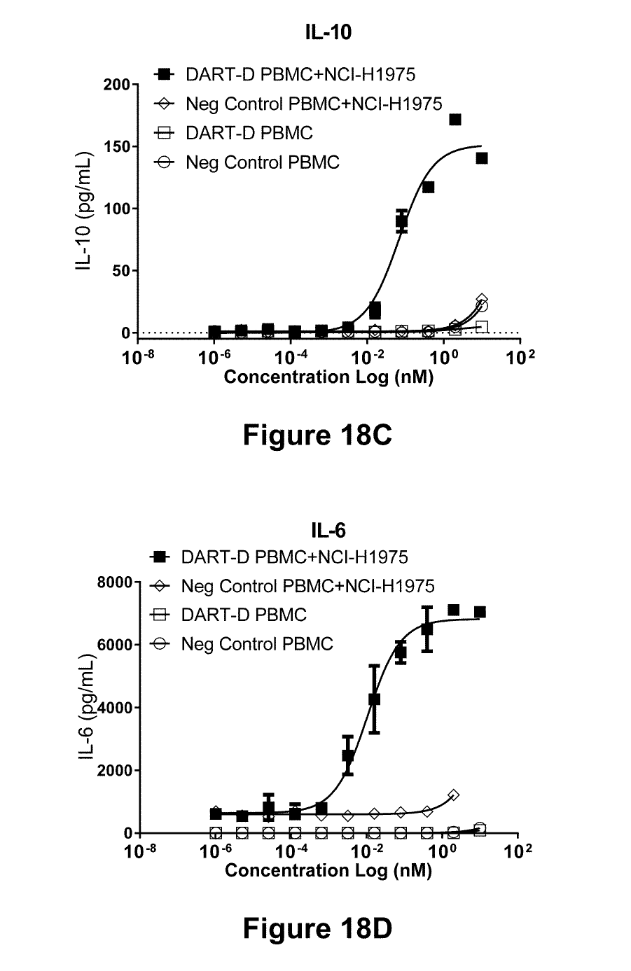

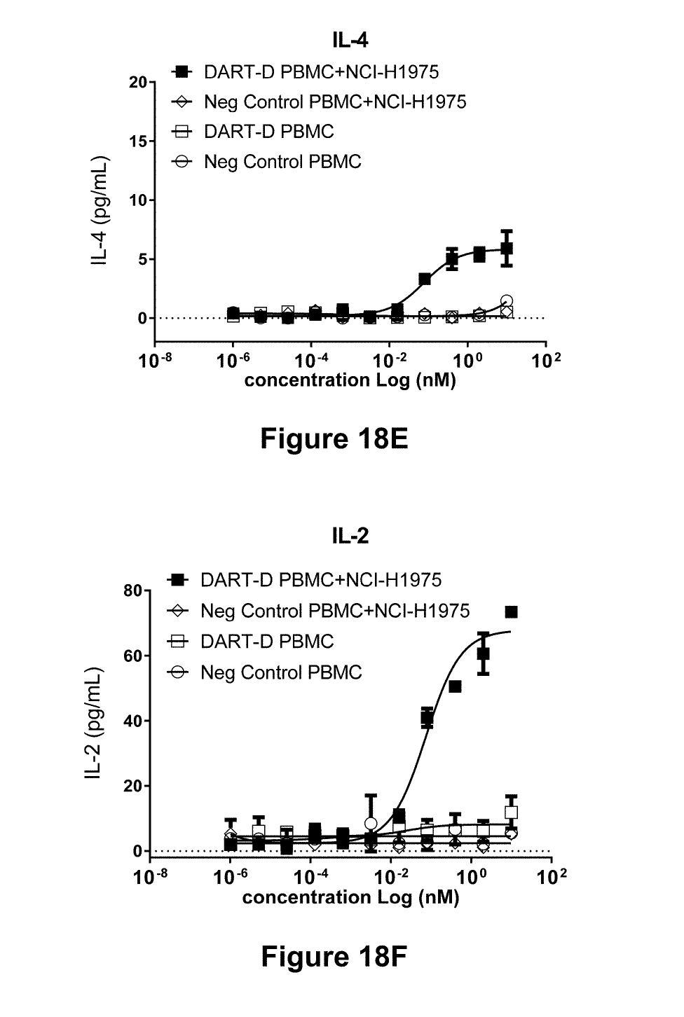

DART-D-mediated T-cell activation correlated with the cytotoxicity of target cells (FIGS. 16A-16B). At all concentrations evaluated, significant DART-D-mediated cytotoxicity was observed in the presence of target cells (FIG. 16A). In contrast, no cytotoxicity was observed when PBMC alone were incubated with DART-D or the control DART in the CTL assay (FIG. 16B). Flow cytometry analyses revealed upregulation of CD69 (FIGS. 17A-17B) and CD25 (FIGS. 17C-17D), T-cell activation markers, on CD4⁺ (FIGS. 17A and 17C) and CD8⁺ T-cell subsets (FIGS. 17B and 17D) in a dose-dependent manner by DART-D in the presence of ROR1-expressing target cells. These data indicate that T-cell activation mediated by the instant bispecific ROR1×CD3 diabodies is dependent upon effector cell-target cell co-engagement. Consistent with T-cell activation markers, dose-dependent increase in levels of all 6 cytokines measured (IFN-γ, TNF-α, IL-10, IL-6, IL-4, and IL-2, FIGS. 18A-18F, respectively) was observed when PBMCs treated with DART-D in the presence of ROR1-expressing target cells (closed symbols). Whereas, no cytokine release was observed when PBMCs alone were treated with DART-D or the control negative control diabody (open symbols).

### Example 4

### In Vivo Studies

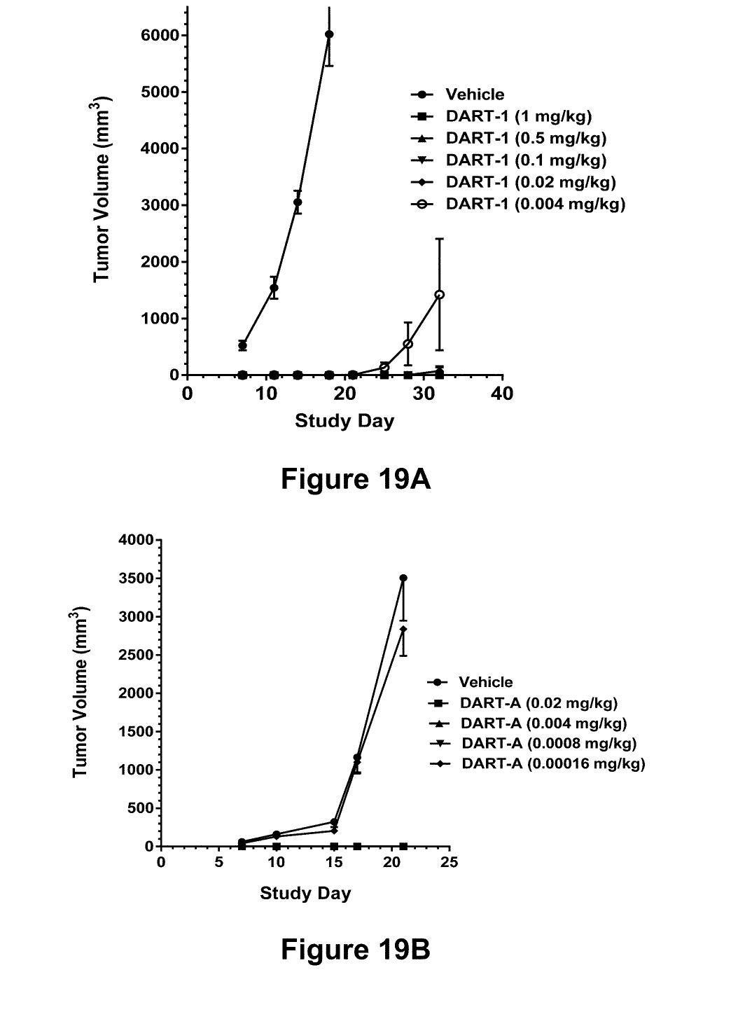

The in vivo activity of the bispecific ROR1×CD3 two and three chain diabodies were examined in several cancer models. In the anti-tumor activity of DART-1 and DART-A was examined in a co-mix HBL-2 mantle cell lymphoma model. Briefly, HBL-2 mantle cell lymphoma cells (5×10⁶) were pre-mixed with activated human T-cells at a ratio of 5:1 and implanted subcutaneously (SQ) into NOD/SCID(NOG) mice (8 female/group) on day 0. Mice were treated, by intravenous (IV) injections once daily for four days starting on day 0, with DART-1 (0.004, 0.02, 0.1, or 1 mg/kg), or vehicle alone in one study, and DART-A (0.00016, 0.0008, 0.004, or 0.02 mg/kg) or vehicle alone in another study. Tumor growth was monitored over the course of the studies. The results of these experiments (FIG. 19A-19B) show that both DART-1 and DART-A were capable of preventing or inhibiting tumor development in this murine xenograft model.

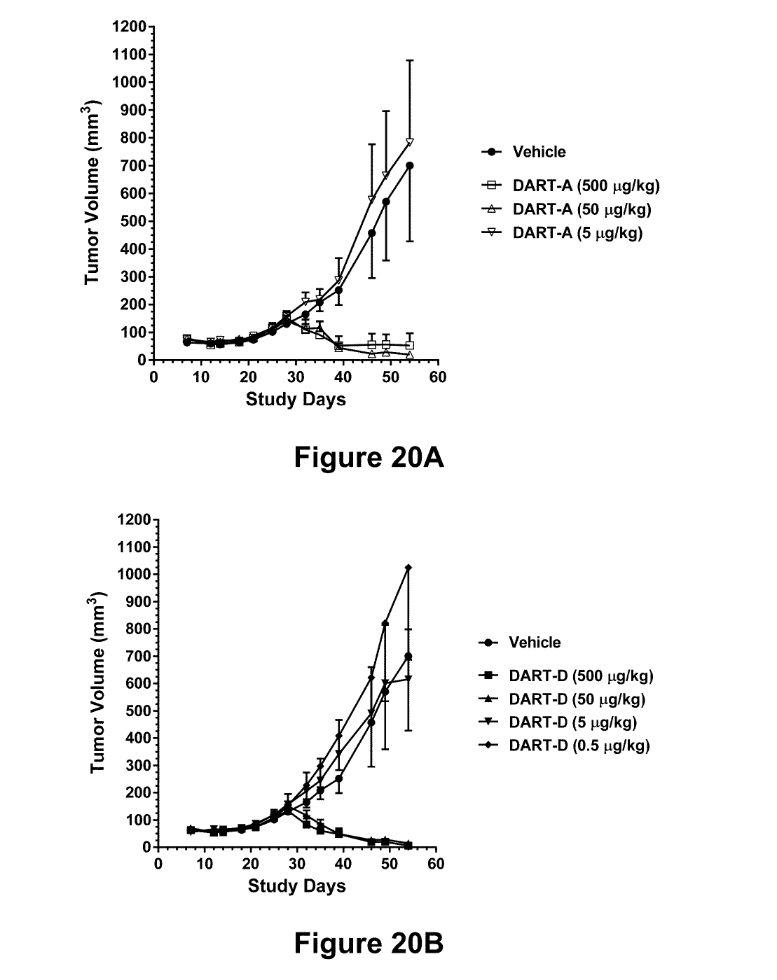

In a further study, the anti-tumor activity of DART-A and DART-D was examined in a PBMC-reconstituted HOP-92 lung adenocarcinoma model. Briefly, HOP-92 cells (5×10⁶) were re-suspended in 50 μL Ham's F12 medium, combined with 50 μL Matrigel, and then implanted by intradermal (ID) injection in MHCl1−/− mice (6-7 female/group) on study day 0 Human PBMCs (1×10⁷ viable cells) were implanted by intraperitoneal (IP) injection (200 μL, Ham's F12 medium) on study day 13. On day 26, animals were randomized into groups and treated with DART-A (5, 50, or 500 μg/kg), DART-D (0.5, 5, 50, or 500 μg/kg) or vehicle alone by IV injections once every 7 days for 5 doses. Tumor volume was monitored over the course of the study. The results of this experiment (FIG. 20A-20B) show that both DART-A and DART-D were capable of preventing or inhibiting tumor development in this murine xenograft model.

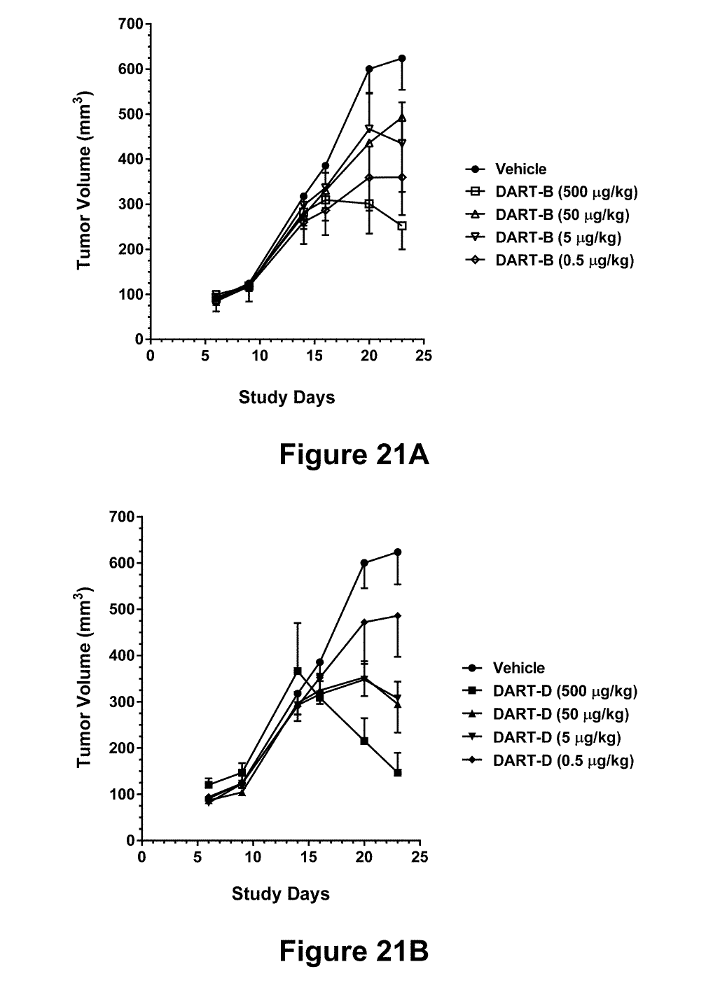

In a further study, the anti-tumor activity of DART-B and DART-D was examined in a PBMC-reconstituted NCI-H1975 lung cancer model. Briefly, NCI-H1975 cells (5×10⁶) were re-suspended in 50 μL Ham's F12 medium, combined with 50 μL Matrigel, and then implanted by ID injection in MHCl1−/− mice (6 female/group) on study day 0. Human PBMCs (1×10⁷ viable cells) were implanted by IP injection (200 μL, Ham's F12 medium) on study day 7. On day 15, animals were randomized into groups and treated with DART-B (0.5, 5, 50, or 500 μg/kg), DART-D (0.5, 5, 50, or 500 μg/kg) or vehicle alone by IV injections once every 7 days for 2 doses. The results of this experiment (FIG. 21A-21B) show that both DART-B and DART-D were capable of preventing or inhibiting tumor development in this murine xenograft model.

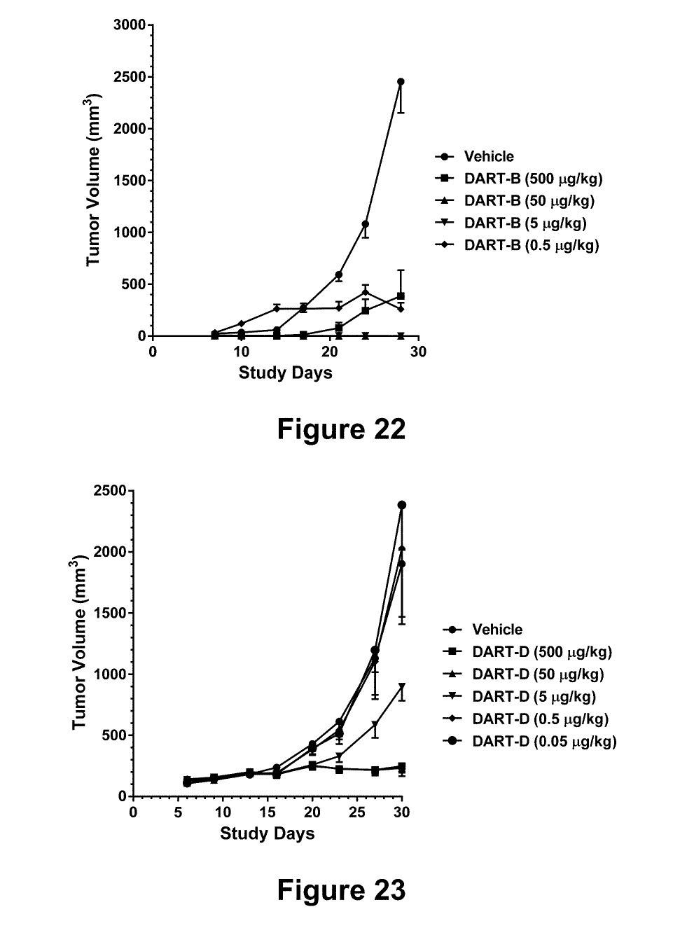

In further study, the anti-tumor activity of DART-B was examined in a co-mix REC1 mantle cancer model. Briefly, REC1 cells (5×10⁶) were pre-mixed with activated human T-cells at a ratio of 5:1 and implanted subcutaneously (SQ) into NOD/SCID(NOG) mice (8 female/group) on day 0. Mice were treated with DART-B (0.5, 5, 50, or 500 μg/kg), or vehicle alone by intravenous (IV) injections once daily for four days starting on day 0. Tumor growth was monitored over the course of the study. The results of this experiment (FIG. 22) show that DART-B were capable of preventing or inhibiting tumor development in this murine xenograft model.

In a further study, the anti-tumor activity of DART-D was examined in a PBMC-reconstituted REC1 mantle cancer model. Briefly, Human PBMCs (1×10⁷ viable cells) were implanted by IP injection (200 Ham's F12 medium) on study day 0. REC1 cells (5×10⁶) were re-suspended in 50 μL Ham's F12 medium, combined with 50 μL Matrigel, and then implanted by ID injection in MHCl1−/− mice (8 female/group) on study day 1. On day 13, animals were randomized into groups and treated with DART-D (0.05, 0.5, 5, 50, or 500 μg/kg) or vehicle alone by IV injections once every 7 days for 4 doses. The results of this experiment (FIG. 23) show that DART-D was capable of preventing or inhibiting tumor development in this murine xenograft model.

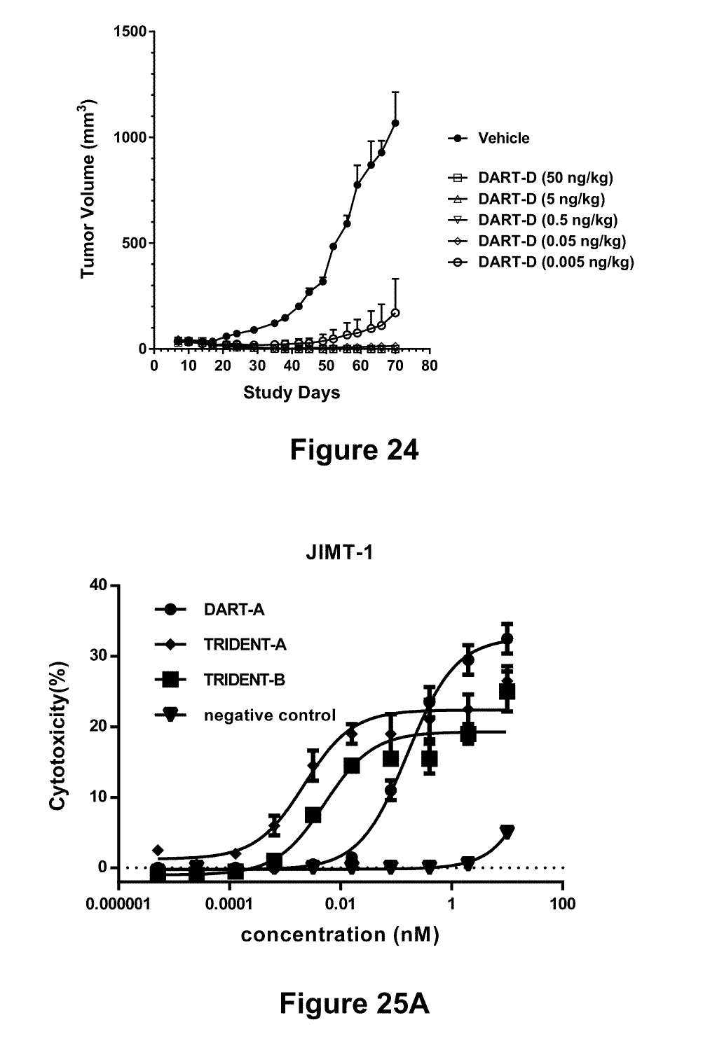

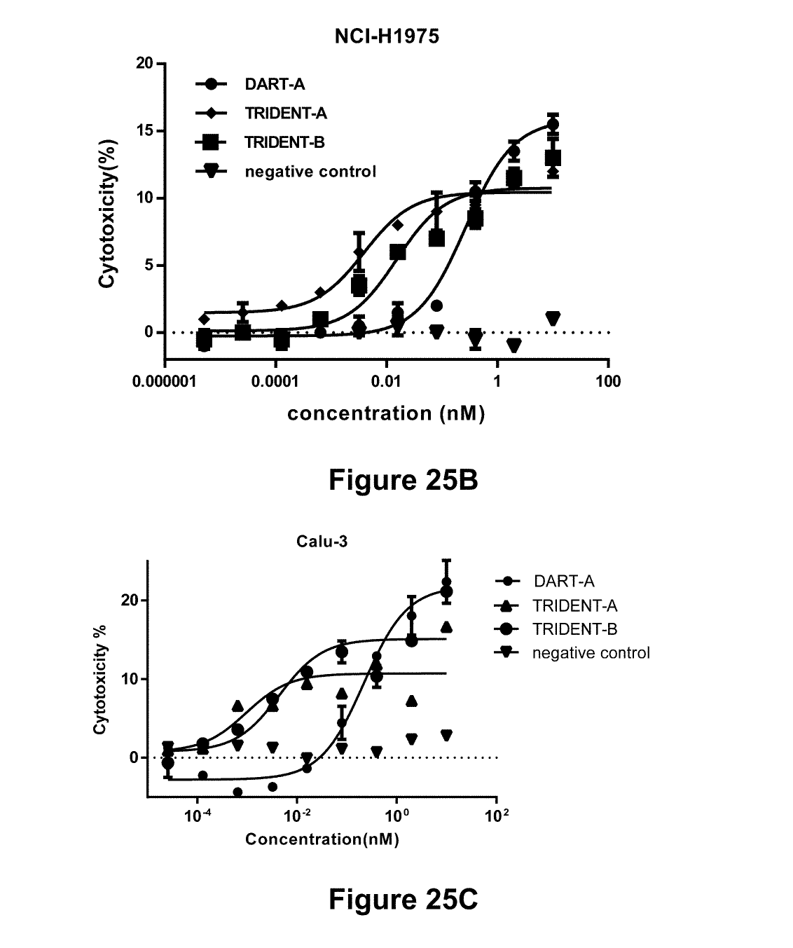

In further study, the anti-tumor activity of DART-D was examined in a co-mix DAOY desmoplastic cerebellar medulloblastoma model. Briefly, DAOY cells (5×10⁶) were pre-mixed with activated human T-cells at a ratio of 5:1 and implanted subcutaneously (SQ) into NOG mice (7 female/group) on day 0. Mice were treated with DART-D (0.005, 0.05, 0.5, 5 or 50 ng/kg), or vehicle alone by intravenous (IV) injections once daily for four days starting on day 0. Tumor growth was monitored over the course of the study. The results of this experiment (FIG. 24) show that DART-D was capable of preventing or inhibiting tumor development in this murine xenograft model.

### Example 5

### Generation of Trispecific Trivalent Binding Molecules

Four trispecific ROR1×CD3×CD8 trivalent binding molecules were generated, each having one binding site specific for ROR1 (comprising parental and/or optimized anti-ROR1-VL and anti-ROR1-VH Domains), one binding site specific for CD3 (comprising the VL and anti-ROR1-VH Domains of CD3 mAb 1 (D65G)), and one binding site specific for CD8 (comprising the VL and anti-ROR1-VH Domains of TRX₂). The trivalent binding molecules TRIDENT-A, having three polypeptide chains and comprising the parental anti-ROR1-VL and anti-ROR1-VH Domains, TRIDENT-B, having four polypeptide chains and comprising the parental anti-ROR1-VL and anti-ROR1-VH Domains, TRIDENT-C, having three polypeptide chains and comprising the optimized anti-ROR1-VL(14) and anti-ROR1-VH(8) Domains and TRIDENT-D, having four polypeptide chains and comprising the optimized anti-ROR1-VL(14) and anti-ROR1-VH(8) Domains are discussed above. The general structure of the polypeptide chains of these three and four chain ROR1×CD3×CD8 trivalent binding molecules is provided in detail above. The particular anti-ROR1-VL and anti-ROR1-VH Domains present in TRIDENT-A, TRIDENT-B, TRIDENT-C, and TRIDENT-D are provided in Table 10.

The binding kinetics of each of the ROR1×CD3×CD8 trivalent binding molecules to ROR1 was investigated using BIACORE® analysis in which each trivalent binding molecule (6.25 to 100 nM) was passed over immobilized shROR1-His essentially as described above. The calculated kₐ, k_{d} and K_{D} from these studies are presented in Table 10, and demonstrate that ROR1×CD3×CD8 trivalent binding molecules comprising the optimized anti-ROR1-VL and anti-ROR1-VH Domains have improved binding affinity. In addition, TRIDENT-A and TRIDENT-B were also shown to be capable of binding to both ROR1 and CD3, demonstrating that ROR1×CD3×CD8 trivalent binding molecules retain dual antigen binding capability.

TABLE 10

| TRIDENT | anti- | anti- | Mutation(s) | kₐ | k_{d} | K_{D} |
|---|---|---|---|---|---|---|
| # | ROR1-VL | ROR1-VH | Kabat | (×10⁵) | (×10⁻⁴) | (nM) |
| A | Parental | Parental | VL: parental | 6.1 | 7.7 | 1.3 |
| | (SEQ ID | (SEQ ID | VH: parental | | | |
| | NO: 6) | NO: 7) | | | | |
| B | Parental | Parental | VL: parental | 5.2 | 8.2 | 1.6 |
| | (SEQ ID | (SEQ ID | VH: parental | | | |
| | NO: 6) | NO: 7) | | | | |
| C | VL(14) | VH(8) | VL: G deleted/R71W | 15 | 2.8 | 0.19 |
| | (SEQ ID | (SEQ ID | VH: F67L N76D | | | |
| | NO: 23) | NO: 31) | A93T | | | |
| D | VL(14) | VH(8) | VL: G deleted/R71W | 14 | 3.6 | 0.26 |
| | (SEQ ID | (SEQ ID | VH: V37I/V63A/ | | | |
| | NO: 23) | NO: 31) | F67L N76D A93T | | | |

The ability of the bispecific ROR1×CD3 three polypeptide chain diabody DART-A and the trispecific ROR1×CD3×CD8 trivalent binding molecules TRIDENT-A and TRIDENT-B to mediate redirected cell killing was assessed using the LDH release assay essentially as described in Example 1. For these studies DART-A, TRIDENT-A, TRIDENT-B or a negative control (a trispecific binding molecule having four polypeptide chains, which binds an irrelevant antigen, CD3, and CD8) were incubated for 24 hours with effector pan T-cells and target tumor cells (JIMT-1 breast cancer cells, NCI-H1975 cells, Calu-3 lung adenocarcinoma cells) at an effector to target ratio of 10:1. Five-fold serial dilutions of DART-A, TRIDENT-A, and TRIDENT-B were utilized. Representative cytotoxicity curves for each target tumor cell type are presented in FIGS. 22A-22C, and the EC50 values are provide in Table 11.

TABLE 11

| | DART-A | TRIDENT-A | TRIDENT-B |
|---|---|---|---|
| | EC50 nM | EC50 nM | EC50 nM |
| JIMT-1 | 0.1612 | 0.0022 | 0.0052 |
| NCI-111975 | 0.2663 | 0.0039 | 0.0153 |
| Calu-3 | 0.1961 | 0.0027 | 0.0044 |

These studies demonstrate that trispecific ROR1×CD3×CD8 trivalent binding molecules have superior ability to mediate redirected cell killing of tumor cells as compared to a bispecific ROR1×CD3 diabody having the same anti-ROR1-VL and anti-ROR1-VH Domains.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A ROR1-binding molecule that comprises a Variable Light Chain (VL) Domain and a Variable Heavy Chain (VH) Domain, wherein the VL Domain has the amino acid sequence of SEQ ID NO:8:
QLVLTQSPSASASLGX₁SVX₂LTCTLSSGHKTDTIDWYQQQPGKAPRYLM
X₃LEGSGSYNKGSGVPDRFX₄SGX₅SSGADX₆YLTISSLQSEDEADYYCG
TDX₇PGNYLFGGGTQLTVLG
wherein X₆ is W, and wherein:
(a) X₁ is S or G, X₂ is K, I or N, X₃ is K or N, X₄ is G or is absent, X₅ is S or I, X₇ is Y or N;
(b) X₁ is S, X₂ is K, X₃ is K, X₄ is G or is absent, X₅ is S, and X₇ is N;
(c) X₁ is S, X₂ is K, X₃ is K, X₄ is G or is absent, X₅ is I, and X₇ is Y;
(d) X₁ is S, X₂ is K, X₃ is K, X₄ is G or is absent, X₅ is I, and X₇ is N; or
(e) X₁ is S, X₂ is K, X₃ is K, X₄ is G or is absent, X₅ is S, and X₇ is Y.

2. The ROR1-binding molecule of claim 1, wherein said VH Domain comprises the amino acid sequence of SEQ ID NO:9:
QEQLVESGGGLVQPGGSLRLSCAASGFTFSDYYMSWX₁RQAPGKGL
EWVATIYPSSGKTYYADSX₂KGRX₃TISSDNAKX₄SLYLQMNSLRAED
TAVYYCX₅RDSYADDAALFDIWGQGTTVTVSS
wherein:
(a) X₁ is V or I, X₂ is V or A, X₃ is L, X₄ is N, D, or Y, and X₅ is A or T;
(b) X₁ is V or I, X₂ is V or A, X₃ is F or L, X₄ is D or Y, and X₅ is A or T;
(c) X₁ is V or I, X₂ is V or A, X₃ is F or L, X₄ is N, D, or Y, and X₅ is T;
(d) X₁ is V or I, X₂ is V or A, X₃ is L, X₄ is N, and X₅ is A;
(e) X₁ is V or I, X₂ is V or A, X₃ is F, X₄ is D, and X₅ is A;
(f) X₁ is V or I, X₂ is V or A, X₃ is F, X₄ is N, and X₅ is T;
(g) X₁ is V or I, X₂ is V or A, X₃ is L, X₄ is D, and X₅ is T;
(h) X₁ is I, X₂ is A, X₃ is F or L, X₄ is N, D or Y, and X₅ is A or T;
(i) X₁ is I, X₂ is A, X₃ is F, X₄ is N, and X₅ is A;
(j) X₁ is I, X₂ is A, X₃ is L, X₄ is N, and X₅ is A;
(k) X₁ is I, X₂ is A, X₃ is F, X₄ is D, and X₅ is A;
(l) X₁ is I, X₂ is A, X₃ is F, X₄ is N, and X₅ is T; or
(m) X₁ is I, X₂ is A, X₃ is L, X₄ is D, and X₅ is T.

3. The ROR1-binding molecule of claim 1, wherein:
(a) said VL comprises the amino acid sequence of SEQ ID NO:11, SEQ ID NO:20, SEQ ID NO:21, SEQ ID NO:22, or SEQ ID NO:23; and
(b) said VH comprises the amino acid sequence of SEQ ID NO:26, SEQ ID NO:24, SEQ ID NO:25, SEQ ID NO:26, SEQ ID NO:30, SEQ ID NO:31, or SEQ ID NO:32.

4. The ROR1-binding molecule of claim 1, wherein said molecule is an antibody or antigen binding fragment thereof.

5. The ROR1-binding molecule of claim 1, wherein said molecule is:
(a) a bispecific antibody; or
(b) a diabody, said diabody being a covalently bonded complex that comprises two, three, four or five polypeptide chains; or
(c) a trivalent binding molecule, said trivalent binding molecule being a covalently bonded complex that comprises three, four, five, or more polypeptide chains.

6. The ROR1-binding molecule of claim 1, wherein said molecule comprises an Fc Region.

7. The ROR1-binding molecule of claim 5, wherein said molecule is a diabody and comprises an Albumin-Binding Domain (ABD).

8. The ROR1-binding molecule of claim 6, wherein said Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

9. The ROR1-binding molecule of claim 8, wherein said modifications that reduces the affinity of the variant Fc Region for an FcγR comprise the substitution of L234A; L235A; or L234A and L235A, wherein said numbering is that of the EU index as in Kabat.

10. The ROR1-binding molecule of claim 8, wherein said modifications that that enhances the serum half-life of the variant Fc Region comprise the substitution of M252Y; M252Y and S254T; M252Y and T256E; M252Y, S254T and T256E; or K288D and H435K, wherein said numbering is that of the EU index as in Kabat.

11. The ROR1-binding molecule of claim 1, wherein said molecule is bispecific and comprises two epitope-binding sites capable of immunospecific binding to an epitope of ROR1 and two epitope-binding sites capable of immunospecific binding to an epitope of a molecule present on the surface of an effector cell.

12. The ROR1-binding molecule of claim 1, wherein said molecule is bispecific and comprises one epitope-binding site capable of immunospecific binding to an epitope of ROR1 and one epitope-binding site capable of immunospecific binding to an epitope of a molecule present on the surface of an effector cell.

13. The ROR1-binding molecule of claim 1, wherein said molecule is trispecific and comprises:
(a) one epitope-binding site capable of immunospecific binding to an epitope of ROR1;
(b) one epitope-binding site capable of immunospecific binding to an epitope of a first molecule present on the surface of an effector cell; and
(c) one epitope-binding site capable of immunospecific binding to an epitope of a second molecule present on the surface of an effector cell.

14. The ROR1-binding molecule of claim 1, wherein said molecule is capable of simultaneously binding to ROR1 and a molecule present on the surface of an effector cell.

15. The ROR1-binding molecule of claim 11, wherein said molecule present on the surface of an effector cell is CD2, CD3, CD8, TCR, or NKG2D.

16. The ROR1-binding molecule of claim 11, wherein said effector cell is a cytotoxic T-cell, or a Natural Killer (NK) cell.

17. The ROR1-binding molecule of claim 11, wherein said molecule present on the surface of an effector cell is CD3.

18. The ROR1-binding molecule of claim 13, wherein said first molecule present on the surface of an effector cell is CD3 and said second molecule present on the surface of an effector cell is CD8.

19. The ROR1-binding molecule of claim 11, wherein said molecule mediates coordinated binding of a cell expressing ROR1 and a cytotoxic T cell.

20. The ROR1-binding molecule of claim 15, wherein said molecule comprises:
(A) the VL Domain of CD3 mAb 1 (SEQ ID NO:75), or one or more CDRs of such VL Domain; and/or
(B) the VH Domain of CD3 mAb 1 (SEQ ID NO:76) or the VH Domain of CD3 mAb 1 (D65G) SEQ ID NO:77), or one or more CDRs of such VH Domains.

21. The ROR1-binding molecule of claim 1, wherein said molecule comprises a first polypeptide chain, a second polypeptide chain and a third polypeptide chain, and wherein:
(a) said a first polypeptide chain comprising SEQ ID NO:98, SEQ ID NO:101, or SEQ ID NO:102;
(b) said second polypeptide chain comprising SEQ ID NO:99, SEQ ID NO:103, or SEQ ID NO:104; and
(c) said third polypeptide chain comprises SEQ ID NO:100.

22. A pharmaceutical composition that comprises an effective amount of the ROR1-binding molecule of claim 1 and a pharmaceutically acceptable carrier, excipient or diluent.

23. A method of treating a disease or condition associated with or characterized by the expression of ROR1, which comprises administering a therapeutically effective amount of the pharmaceutical composition of claim 22 to a recipient in need thereof.

24. The method of claim 23, wherein said disease or condition associated with or characterized by the expression of ROR1 is cancer.

25. The method of claim 24, wherein said cancer is characterized by the presence of a cancer cell selected from the group consisting of a cell of: an adrenal gland tumor, an AIDS-associated cancer, an alveolar soft part sarcoma, an astrocytic tumor, an adrenal cancer, a bladder cancer, a bone cancer, a brain and spinal cord cancer, a metastatic brain tumor, a B-cell cancer, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gastric cancer, a gestational trophoblastic disease, a germ cell tumor, a head and neck cancer, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

26. The method of claim 24, wherein said cancer is selected from the group consisting: adrenal cancer, bladder cancer, breast cancer, colorectal cancer, gastric cancer, glioblastoma, kidney cancer, non-small-cell lung cancer, acute lymphocytic leukemia, acute myeloid leukemia, chronic lymphocytic leukemia, chronic myeloid leukemia, hairy cell leukemia, Burkett's lymphoma, diffuse large B cell lymphoma, follicular lymphoma, mantle cell lymphoma, marginal zone lymphoma, non-Hodgkin's lymphoma, small lymphocytic lymphoma, multiple myeloma, melanoma, ovarian cancer, pancreatic cancer, prostate cancer, skin cancer, renal cell carcinoma, testicular cancer, and uterine cancer.
